(19) United States
(12) Patent Application Publication
(10) Pub. No.: US 2017/0313669 A1
(43) Pub. Date: Nov. 2, 2017

(54) LACTONE COMPOUNDS AND METHODS OF MAKING AND USING SAME

(71) Applicants: THE SCRIPPS RESEARCH INSTITUTE, La Jolla, CA (US); THE UNIVERSITY OF CONNECTICUT, Farmington, CT (US)

(72) Inventors: Benjamin F. CRAVATT, La Jolla, CA (US); Siddhesh S. KAMAT, Escondido, CA (US); William H. PARSONS, San Diego, CA (US); Amy R. HOWELL, Tolland, CT (US); Kaddy CAMARA, Brighton, MA (US); Amy ANDERSON, Storrs, CT (US)

(21) Appl. No.: 15/520,389

(22) PCT Filed: Oct. 27, 2015
(86) PCT No.: PCT/US2015/057490

§ 371 Date: Apr. 19, 2017

(51) Int. Cl. C07D 305/12; A61K 31/337; A61K 31/365

(52) U.S. Cl. CPC C07D 305/12; A61K 31/365; A61K 31/337; A61K 2121/00

(57) ABSTRACT

Provided herein are lactone compounds and pharmaceutical compositions comprising said compounds. The subject compounds and compositions are useful as inhibitors of serine hydrolases, such as ABHD16A. Furthermore, the subject compounds and compositions may be useful for the treatment of, for example, PHARC and other neuroinflammatory diseases.

### CROSS-REFERENCE

This application claims the benefit of U.S. provisional application No. 62/069,004, filed Oct. 27, 2014, which is incorporated by reference herein in its entirety.

### BACKGROUND

Lysophospholipids serve as critical small-molecule transmitters that act on both receptors and channels to regulate many facets of mammalian biology and disease. Lysophosphatidic acid (LPA) and sphingosine-1-phosphate (S1P) are two lysophospholipids that produce their biological effects principally through distinct sets of G-protein coupled receptors (GPCRs). Lysophosphatidylserines (lyso-PSs) are an additional class of signaling lysophospholipids with activity on Toll-like receptors (TLRs) and GPCRs that are highly enriched in immune cells and implicated in human autoimmune disorders. Lyso-PSs regulate macrophage activation and clearance of apoptotic cells, leukemia cell stimulation, mast cell degranulation, and chemotactic movements of human glioma cells.

Enzymes that produce and degrade lysophospholipids represent nodal points for regulating cell signaling and can be targeted by genetic and/or pharmacological methods to perturb lysophospholipid pathways to determine their functional roles in physiology and disease. While the enzymes that regulate more established lysophospholipids, such as LPA and S1P, are understood, the metabolic pathways that generate and terminate lyso-PS signals remain poorly characterized.

The serine hydrolase ABHD12 acts as a major lyso-PS lipase in the mouse brain. Deleterious mutations in the ABHD12 gene cause the rare autosomal recessive neurological disorder PHARC (Polyneuropathy, Hearing loss, Ataxia, Retinitis pigmentosa, and Cataract). PHARC is marked by polymodal sensory and motor defects, which are linked to cerebellar atrophy, peripheral neuropathy, early onset of cataract, blindness, and hearing loss, as well as demyelination of sensorimotor neurons. PHARC symptoms progress slowly, first appearing in late childhood or early teens and progressively worsening with age.

ABHD12^{-/-} mice exhibit auditory and motor deficits coupled with elevated brain lyso-PS content and heightened neuroinflammation, implicating deregulated lyso-PS signaling as a contributory factor to PHARC-like syndromes. Elucidating the enzymes that produce lyso-PSs could thus provide targets for control over these lipid signals to determine their functions in physiology and disease, as well as to potentially treat disorders caused by aberrant lyso-PS activity. While a handful of enzymes have been found to hydrolyze phosphatidylserine (PS) to lyso-PS in vitro, the contribution that these proteins, or others, make to lyso-PS production in vivo remains unknown.

ABHD16A was originally named HLA-B associated transcript 5 (BAT5), a designation that reflects the location of the ABHD16A gene within the HLA-B region of the human genome, which harbors several other genes that code for proteins that play important roles in immunology (e.g., TNF). Indeed, it had been speculated over twenty years ago that the protein products of these genes could be involved in aspects of immunity. Despite this intriguing hypothesis, ABHD16A (BAT5) has remained unannotated in terms of its biochemical and cellular function.

### BRIEF SUMMARY OF THE INVENTION

Disclosed herein are lactone compounds that are inhibitors of ABHD16A, and their use as medicinal agents, processes for their preparation, and pharmaceutical compositions comprising them. The disclosure also provides for the use of disclosed compounds as medicaments and/or in the manufacture of medicaments for the inhibition of ABHD16A activity in warm-blooded animals such as humans.

One embodiment provides a compound of Formula (I):

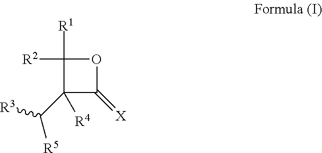

wherein:
X is O or CH₂;
R¹ is alkyl or -alkylene-(optionally substituted aryl);
R² is H;
R³ is a optionally substituted aryl, optionally substituted aralkyl, or optionally substituted alkyl; and
R⁴ and R⁵ are both H; or R⁴ and R⁵ together form a direct bond to provide a double bond;
or a solvate, hydrate, tautomer, N-oxide, stereoisomer, or pharmaceutically acceptable salt thereof.

In some embodiments of a compound of Formula (I), X is O. In some embodiments of a compound of Formula (I), X is CH₂. In some embodiments of a compound of Formula (I), R⁴ and R⁵ are both H. In some embodiments of a compound of Formula (I), R⁴ and R⁵ together form a direct bond to provide a double bond.

Another embodiment provides a compound of Formula (II):

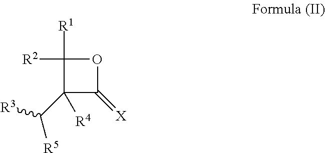

wherein:
X is O or CH₂;
R¹ is alkyl or -alkylene-(optionally substituted aryl);
R² is H;
R³ is optionally substituted aryl, —(CH₂)ₙ—CH₃, —(CH₂)ₙ-(optionally substituted aryl), —(CH₂)ₙ—C(O)NR⁶R⁷, or —(CH₂)ₙ—SO₂NR⁶R⁷;
R⁴ and R⁵ are both H; or R⁴ and R⁵ together form a direct bond to provide a double bond;
R⁶ is H or alkyl;
R⁷ is H or alkyl; and
n is 1, 2, 3, 4, 5, or 6;
or a solvate, hydrate, tautomer, N-oxide, stereoisomer, or pharmaceutically acceptable salt thereof.

In some embodiments of a compound of Formula (II), R¹ is alkyl or -alkylene-(optionally substituted phenyl). In some embodiments of a compound of Formula (II), R³ is optionally substituted phenyl, —(CH₂)ₙ—CH₃, —(CH₂)ₙ-(optionally substituted phenyl), —(CH₂)ₙ—C(O)NR⁶R⁷, or —(CH₂)ₙ—SO₂NR⁶R⁷. In some embodiments of a compound of Formula (II), R⁶ and R⁷ are both H. In some embodiments of a compound of Formula (II), R⁴ and R⁵ are both H. In some embodiments of a compound of Formula (II), R⁴ and R⁵ together form a direct bond to provide a double bond. In some embodiments of a compound of Formula (II), X is O. In some embodiments of a compound of Formula (II), X is CH₂.

Another embodiment provides a compound of Formula (III):

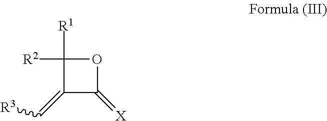

wherein:
X is O or CH₂;
R¹ is alkyl or -alkylene-(optionally substituted aryl);
R² is H;
R³ is optionally substituted aryl, —(CH₂)ₙ—CH₃, —(CH₂)ₙ-(optionally substituted aryl), —(CH₂)ₙ—C(O)NR⁶R⁷, or —(CH₂)ₙ—SO₂NR⁶R⁷;
R⁶ is H or alkyl;
R⁷ is H or alkyl; and
n is 1, 2, 3, 4, 5, 6, 7, 8, 9, 10, 11, 12, 13, 14, 15, 16, 17, 18, 19, or 20;
or a solvate, hydrate, tautomer, N-oxide, stereoisomer, or pharmaceutically acceptable salt thereof.

In some embodiments of a compound of Formula (III), R¹ is alkyl or -alkylene-(optionally substituted phenyl). In some embodiments of a compound of Formula (III), R³ is optionally substituted phenyl, —(CH₂)ₙ—CH₃, —(CH₂)ₙ-(optionally substituted phenyl), —(CH₂)ₙ—C(O)NR⁶R⁷, or —(CH₂)ₙ—SO₂NR⁶R⁷. In some embodiments of a compound of Formula (III), R⁶ and R⁷ are both H. In some embodiments of a compound of Formula (III), X is O. In some embodiments of a compound of Formula (III), X is CH₂.

Another embodiment provides a compound of Formula (IV):

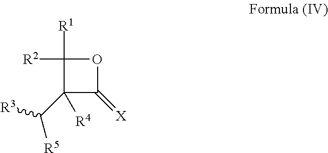

wherein:
X is O or CH₂;
R¹ is alkyl or -alkylene-(phenyl);
R² is H;
R³ is phenyl, —(CH₂)ₙ—CH₃, —(CH₂)ₙ-(phenyl), —(CH₂)ₙ—C(O)NH₂, or —(CH₂)ₙ—SO₂NH₂;
R⁴ and R⁵ are both H; or R⁴ and R⁵ together form a direct bond to provide a double bond; and
n is 1, 2, 3, 4, 5, or 6;
or a solvate, hydrate, tautomer, N-oxide, stereoisomer, or pharmaceutically acceptable salt thereof.

In some embodiments of a compound of Formula (IV), R⁴ and R⁵ are both H. In some embodiments of a compound of Formula (IV), R⁴ and R⁵ together form a direct bond to provide a double bond. In some embodiments of a compound of Formula (IV), X is O. In some embodiments of a compound of Formula (IV), X is CH₂.

Another embodiment provides a compound of Formula (V):

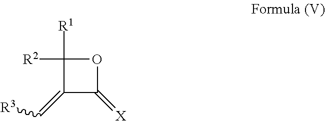

wherein:
X is O or CH₂;
R¹ is alkyl or -alkylene-(phenyl);
R² is H;
R³ is phenyl, —(CH₂)ₙ—CH₃, —(CH₂)ₙ-(phenyl), —(CH₂)ₙ—C(O)NH₂, or —(CH₂)ₙ—SO₂NH₂;
n is 1, 2, 3, 4, 5, 6, 7, 8, 9, 10, 11, 12, 13, 14, 15, 16, 17, 18, 19, or 20;
or a solvate, hydrate, tautomer, N-oxide, stereoisomer, or pharmaceutically acceptable salt thereof

In some embodiments of a compound of Formula (V), X is O. In some embodiments of a compound of Formula (V), X is CH₂.

Another embodiment provides a compound of Formula (VI):

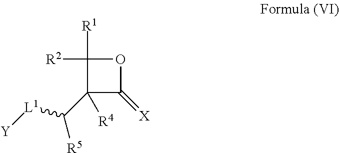

wherein:
X is O or CH₂;
R¹ is alkyl or -alkylene-(optionally substituted aryl);
R² is H;
R⁴ and R⁵ are both H; or R⁴ and R⁵ together form a direct bond to provide a double bond;
L¹ is a linker; and Y is a fluorophore;
or a solvate, hydrate, tautomer, N-oxide, stereoisomer, or pharmaceutically acceptable salt thereof.

In some embodiments of a compound of Formula (VI), X is O.

In some embodiments of a compound of Formula (VI), X is CH₂.

In some embodiments of a compound of Formula (VI), R⁴ and R⁵ are both H.

In some embodiments of a compound of Formula (VI), R⁴ and R⁵ together form a direct bond to provide a double bond.

In some embodiments of a compound of Formula (VI), Y is selected from fluorescein, 6-FAM, rhodamine, Texas Red, California Red, iFluor594, carboxytetramethylrhodamine, tetramethylrhodamine, a carboxyrhodamine, carboxyrhodamine 6F, carboxyrhodol, carboxyrhodamine 110, Cascade Blue, Cascade Yellow, coumarin, Cy2®, Cy3®, Cy3.5®, Cy5®, Cy5.5®, Cy7®, Cy-Chrome, DyLight 350, DyLight 405, DyLight 488, DyLight 549, DyLight 594, DyLight 633, DyLight 649, DyLight 680, DyLight 750, DyLight 800, phycoerythrin, PerCP (peridinin chlorophyll-a Protein), PerCP-Cy5.5, JOE (6-carboxy-4′,5′-dichloro-2′,7′-dimethoxyfluorescein), NED, ROX (5-(and-6-)-carboxy-X-rhodamine), HEX, Lucifer Yellow, Marina Blue, Oregon Green 488, Oregon Green 500, Oregon Green 514, Alexa Fluor® 350, Alex Fluor® 430, Alexa Fluor® 488, Alexa Fluor® 532, Alexa Fluor® 546, Alexa Fluor® 568, Alexa Fluor® 594, Alexa Fluor® 633, Alexa Fluor® 647, Alexa Fluor® 660, Alexa Fluor® 680, 7-amino-4-methylcoumarin-3-acetic acid, BODIPY® FL, BODIPY® FL-Br₂, BODIPY® 530/550, BODIPY® 558/568, BODIPY® 630/650, BODIPY® 650/665, BODIPY® R6G, BODIPYR TMR, BODIPY® TR, conjugates thereof, derivatives thereof, analogs thereof, and combinations thereof.

Another embodiment provides a compound selected from:

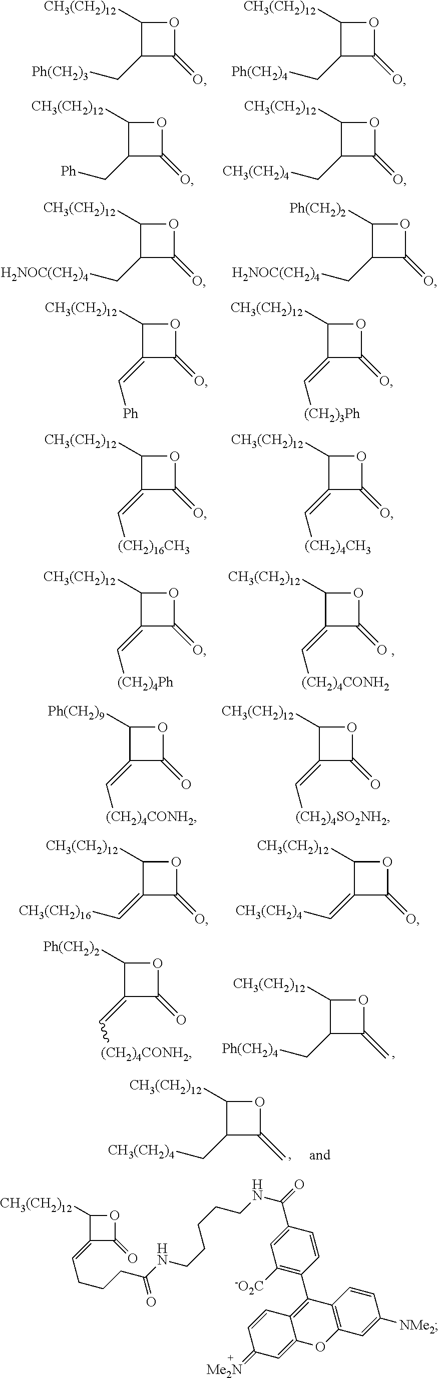

or a solvate, hydrate, tautomer, N-oxide, stereoisomer, or a pharmaceutically acceptable salt thereof.

Further embodiments provided herein include combinations of one or more of the particular embodiments set forth above.

In some embodiments, the compound disclosed herein has the structure provided in Table 1.

Another embodiment provides a pharmaceutical composition comprising a lactone compound described herein, or a solvate, hydrate, tautomer, N-oxide, stereoisomer, or a pharmaceutically acceptable salt thereof, and at least one pharmaceutically acceptable excipient.

Another embodiment provides a method of treating PHARC, comprising administering to a subject in need thereof a therapeutically effective amount of an ABHD16A inhibitor. In some embodiments, the ABHD16A inhibitor is a lactone compound described herein.

Another embodiment provides a method of treating a neuroinflammatory disease, comprising administering to a subject in need thereof a therapeutically effective amount of an ABHD16A inhibitor. In some embodiments, the ABHD16A inhibitor is a lactone compound described herein.

Another embodiment provides a method of reducing cellular and/or secreted levels of pro-inflammatory lysophosphatidylserine lipids in mammalian cells, the method comprising contacting the mammalian cells with an ABHD16A inhibitor. In some embodiments, the ABHD16A inhibitor is a lactone compound described herein. In some embodiments, the method is an in vivo method. In some methods, the method is an in vitro method.

Another embodiment provides a method of inhibiting ABHD16A in mammalian cells, the method comprising contacting the mammalian cells with a compound described herein. In some embodiments, the method is an in vivo method. In some methods, the method is an in vitro method.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1a shows competitive gel-based ABPP (activity-based protein profiling) of β-lactone probe set with serine hydrolase targets from mouse brain membrane proteome.

FIG. 1b shows competitive gel-based ABPP of β-lactone probe set with serine hydrolase targets from COLO205 colon cancer cells membrane proteome.

FIG. 1c shows ABPP-SILAC analysis of the COLO205 membrane proteome treated with compound 8b.

FIG. 2 shows biochemical characterization of a PS lipase activity in mouse brain. (a) Mouse brain proteomes were treated with DMSO or FP-rhodamine and then assayed for PS lipase activity with a C18:0/C18:2 PS substrate. (b) Inhibition of mouse brain membrane fraction PS lipase activity by different FP-probes. (c) BioGPS gene expression profile for mouse Abhd16a in various regions of the brain and nervous system.

FIG. 3 shows identification of ABHD16A as a PS lipase. (a) PS lipase activity of mouse brain soluble and membrane proteomes after pre-treatment with DMSO or FP-rhodamine. (b) Concentration-dependent inhibition of brain membrane PS lipase activity by tetrahydrolipstatin (THL). (c) ABPP gel of the membrane proteomes (1 mg protein/mL) of the different mouse brain regions treated with FP-rhodamine. (d) PS lipase activity of membrane proteomes from mouse brain regions. (e) ABPP-gel of membrane proteomes from mock- and ABHD16A-transfected HEK293T cells, showing robust expression and activity of the recombinant mouse ABHD16A enzyme. (f) PS lipase activity of membrane proteomes of mock- and ABHD16A-transfected HEK293T cells. (g) In vitro lipid substrate hydrolysis assays for membrane proteomes of mock and murine ABHD16A transfected HEK293T proteomes.

FIG. 4 shows recombinant expression and activity of ABHD16A. (a) ABPP gel showing the expression and activity of mouse and human ABHD16A in transfected HEK293T cell proteomes compared to a mock-transfected HEK293T cell proteome. (b) PS-lipase activity of mock-, mouse ABHD16A-, and human ABHD16A-transfected HEK293T membrane proteomes measured using a C18:0/C18:2 PS substrate (100 μM).

FIG. 5 shows screening of α-alkylidene-β-lactones against human ABHD16A by competitive ABPP.

FIG. 6 shows identification of an ABHD16A inhibitor and a paired inactive control probe. (a) Structures of α-methylene-β-lactone probes—the ABHD16A inhibitor KC01 (compound 13b) and the inactive control probe KC02 (compound 14). KC02 was synthesized and assayed as a 4:1 mixture of Z:E isomers. (b) Competitive ABPP gels showing the concentration-dependent inhibition of ABHD16A by KC01, but not KC02, in ABHD16A-transfected HEK293T cell proteomes. (c) Concentration-dependent inhibition of the PS lipase activity of the membrane proteome of human ABHD16A-transfected HEK293T cells by KC01 and KC02. Data represent mean values±s.e.m. for three biological replicates, and the 95% interval for the reported IC₅₀ value of KC01 is 69-105 nM. (d, e) ABPP-SILAC analysis of serine hydrolase activities in COLO205 colon cancer cells treated in situ with KC01 (d) or KC02 (e) for 4 h. Data represent mean values±s.d. for two biological replicates, where each biological replicate value corresponds to the median SILAC ratio for the total quantified peptides observed for each enzyme (minimum of three unique peptides per enzyme required for analysis).

FIG. 7 shows concentration-dependent inhibition of mouse ABHD16A activity. (a) The indicated concentrations of KC01 and KC02 were incubated with mouse ABHD16A-transfected HEK293T cell proteomes and then samples were treated with FP-rhodamine and analyzed by gel-based ABPP. (b) Concentration dependent inhibition of the PS-lipase activity of mouse ABHD16A by KC01.

FIG. 8 shows inhibitory activity of KC01 analogues against recombinant human ABH16A as measured by competitive ABPP. (a) Structures of KC01 analogues. (b) Varying concentrations of the KC01 analogues were incubated against human ABHD16A-transfected HEK293T proteomes.

FIG. 9 shows inhibitory activity of KC01 in the human K562 leukemia cell line as measured by competitive ABPP.

FIG. 10 shows the PS lipase activity of cancer cells is blocked by KC01, but not KC02.

FIG. 11 shows disruption of ABHD16A reduces the lyso-PS content of human cells. (a, b) Cellular (a) and secreted (b) concentrations of lyso-PS from COLO205 colon cancer cells treated in situ with inhibitors (KC01 or KC02, 1 μM) or DMSO for 4 h. (c) Spectral count values of serine hydrolase activities of the membrane proteomes of mock (uninfected), control-shRNA, KD_1-shRNA and KD_2-shRNA K562 leukemia cell line models as measured by ABPP-MudPIT. Inset shows a blow-up of the ABHD16A spectral counts. (d) PS lipase activities of membrane proteomes from the indicated K562 cell lines. (e) Cellular concentrations of lyso-PS from the indicated K562 cell lines. Data represent mean values±s.e.m.; N=8 per group. Student's t-test: ** p<0.0005, *** p<0.0001 for KD_1 or KD_2 cells versus control or uninfected cells. (f) Secreted concentrations of lyso-PS from the indicated lymphoblast cell lines (LCLs), which includes LCLs from a PHARC subject (ABHD12−/−), the subject's mother (ABHD12+/−), the subject's brother (ABHD12+/+), and two control subjects (ABHD12+/+). Data represent mean values±s. e. m for four biological replicates. Student's t-test: *** p<0.0001 for the PHARC subject LCL versus control 1 or control 2 LCLs. (g) Concentrations of secreted C18:0 lyso-PS from the indicated LCLs treated with DMSO, KC01, or KC02 (1 μM inhibitors, 4 h). Data represent mean values±s. e. m for four biological replicates. Student's t-test: ** p<0.0005, *** p<0.0001 for KC01-treated versus DMSO-treated LCLs.

FIG. 12 shows lyso-PS content of K562 cells treated with KC01 or KC02.

FIG. 13 shows lyso-PS content of MCF7 cells treated with KC01 or KC02.

FIG. 14 shows PS content of K562 cells expressing different shRNA knockdown constructs.

FIG. 15 shows lyso-PS lipase and PS lipase activities of human lymphoblast cell lines (LCLs). (a) Lyso-PS lipase activity of membrane proteomes from indicated LCLs. (b) PS lipase activity of membrane proteomes from indicated LCLs.

FIG. 16 shows treatment of human LCLs with KC01 and KC02. (a) Gel-based ABPP analysis of the membrane proteome of indicated LCLs following treatment with inhibitors (KC01 or KC02, 1 μM) or DMSO for 4 h (in situ). In addition to showing the selective loss of ABHD16A in KC01-treated cells, the ABPP gel also shows selective loss of ABHD12 in PHARC LCL compared to other LCLs. Gel-based ABPP experiments were performed in triplicate with consistent results. (b, c) Concentrations of secreted (b) and cellular (c) lyso-PS from PHARC LCL line (b) or PHARC and indicated Control lines (c) treated with inhibitors (KC01 or KC02, 1 μM) or DMSO for 4 h. For (b, c), Data represent mean values±s.e.m. for four biological replicates. Student's t-test: ** p<0.005, *** p<0.0001 for KC01- versus DMSO-treated samples

FIG. 17 shows protease protection assays for ABHD16A and ABHD12: (a) gel-based ABPP results for ABHD16A; (b) anti-FLAG blotting results for ABHD16A; and (c) gel-based ABPP results for ABHD12.

FIG. 18 shows the effect of PNGaseF treatment on SDS-PAGE migration of ABHD16A and ABHD12. Mouse brain membrane proteome (1 mg/mL) (a) or ABHD16A-transfected HEK293T proteome (1 mg/mL) (b) was treated with FP-rhodamine (2 μM, 30 min, 37° C.) and then exposed to PNGaseF. No change in the gel migration of endogenous brain (a, see inset) or recombinant (b) ABHD16A was observed in the presence of PNGaseF. In contrast, several other serine hydrolases showed changes in migration, including the lumenally oriented, glycoproteins AADACL1 and ABHD12 (a). Gel-based ABPP experiments were performed in triplicate with consistent results.

FIG. 19 shows the gene expression profile of ABHD16A and ABHD12 in peritoneal macrophages, in particular, the BioGPS-derived gene expression profiles of ABHD16A and ABHD12 in mouse peritoneal macrophages following stimulation with lipopolysaccharide (LPS) for 0, 1, and 7 h. Original data can be found at: http://biogps.org/.

FIG. 20 shows ABHD16A-mediated regulation of lyso-PS and pro-inflammatory cytokines in peritoneal macrophages. (a) Normalized spectral count ratios for serine hydrolase activities detected in an ABPP-MudPIT analysis of the membrane fractions of thioglycollate-elicited peritoneal macrophages treated with vehicle (PBS) or LPS (5 μg/mL for 7 h). Data represent mean values±s.e.m. for three biological replicates. (b) PS lipase activity of membrane proteomes of peritoneal macrophages treated with inhibitors (KC01 or KC02, 1 μM) or DMSO for 30 min at 37° C. Data represent mean values±s.e.m. for three biological replicates. (c) Lyso-PS lipase activity of membrane proteomes of peritoneal macrophages treated with DMSO, THL (10 μM, 30 min 37° C.). For (b) and (c), vehicle- and LPS-treated macrophages were prepared as described in (a), and data represent mean values±s.e.m. for three biological replicates. Student's t-test: ** p<0.0005 for KC01- versus DMSO-treated samples; *** p<0.0001, for LPS-versus vehicle-treated samples. (d, e) Concentrations of cellular (d) and secreted (e) lyso-PS from thioglycollate-elicited peritoneal macrophages, treated with vehicle (PBS) or LPS (5 μg/mL) for 7 h. Data represent mean values±s.e.m. for four biological replicates, Student's t-test: * p<0.05, ** p<0.0005 for LPS-treated versus vehicle-treated groups. (f, g) Concentrations of secreted lyso-PS (f) and TNF- (g) for thioglycollate-elicited peritoneal macrophages treated with inhibitors (1 μM) or DMSO for 4 h followed by stimulation with LPS (5 μg/mL, 7 h). Data represent mean values±s.e.m. for four biological replicates. Student's t-test: ** p<0.0005, *** p<0.0001 for KC01-treated versus DMSO-treated groups.

FIG. 21 shows the effect of KC01 and KC02 on cellular lyso-PS content of mouse macrophages, in particular, the concentration of cellular lyso-PS lipids from thioglycollate-elicited peritoneal mouse macrophages treated with inhibitors (KC01 or KC02, 1 μM) or DMSO for 4 h, followed by stimulation with vehicle (PBS, a) or LPS (5 μg/mL, b) for 7 h.

Details for sample preparation and lipid measurements analysis can be found herein. Data represent mean values±s.e.m. for four biological replicates. Student's t-test: ** p<0.0005, *** p<0.0001 for KC01- versus DMSO-treated samples.

FIG. 22 shows the effect of KC01 and KC02 on secreted lyso-PS content of mouse macrophages, in particular, the concentration of secreted lyso-PS lipids from thioglycollate-elicited peritoneal mouse macrophages treated with inhibitors (KC01 or KC02, 1 μM) or DMSO for 4 h, followed by treatment with PBS for 7 h. See FIG. 20f for a similar study performed on macrophages treated with LPS for 7 h. Details for sample preparation and lipid measurements analysis can be herein. Data represent mean values±s.e.m. for four biological replicates. Student's t-test: ** p<0.0005 for KC01- versus DMSO-treated samples.

FIG. 23 shows the effect of KC01 and KC02 on secreted cytokines from mouse macrophages, in particular, the concentration of secreted cytokines from thioglycollate-elicited peritoneal mouse macrophages treated with inhibitors (KC01 or KC02, 1 μM) or DMSO for 4 h followed by treatment with vehicle (PBS) or LPS (5 μg/mL) for 7 h. Data represent mean values±s.e.m. for four biological replicates. Student's t-test: * p<0.05, *** p<0.0001 for KC01- versus DMSO-treated samples.

FIG. 24 shows how the interplay between ABHD12 and ABHD16A regulates lyso-PS and cytokine release from macrophages. (a, b) Concentrations of secreted cytokines from ABHD12^{+/+} and ABHD12^{-/-} thioglycollate-elicited peritoneal macrophages that were either unstimulated (PBS) (a) or stimulated (b) with LPS (5 μg/mL, 7 h). Data represent mean values±s.e.m. for four biological replicates. Student's t-test: * p<0.05 for ABHD12^{-/-} and ABHD12^{+/+} groups. (c) Concentrations of secreted lyso-PS from ABHD12^{+/+} and ABHD12^{-/-} macrophages treated with vehicle (PBS) or LPS (5 μg/mL) for 7 h. Data represent mean values±s.e.m. for four biological replicates. Student's t-test: * p<0.05, ** p<0.0005 for ABHD12^{-/-} and ABHD12^{+/+} groups (d, e) Concentrations of secreted lyso-PS (d) and TNF-α (e) from ABHD12^{-/-} macrophages treated with inhibitors (KC01 or KC02, 1 μM) or DMSO for 4 h followed by treatment with vehicle (PBS) or LPS (5 μg/mL) for 7 hours. Data represent mean values±s.e.m. for four biological replicates. Student's t-test: ** p<0.0005, *** p<0.0001 for KC01-treated versus DMSO-treated groups.

FIG. 25 shows the effect of KC01 and KC02 on cellular lyso-PS content of ABHD12^{-/-} macrophages, in particular, the concentration of cellular lyso-PS lipids from thioglycollate-elicited peritoneal mouse macrophages treated with inhibitors (KC01 or KC02, 1 μM) or DMSO for 4 h, followed by stimulation with vehicle (PBS, a) or LPS (5 μg/mL, b) for 7 h. Details for sample preparation and lipid measurements analysis can be found herein. Data represent mean values±s.e.m. for four biological replicates. Student's t-test: ** p<0.0005, *** p<0.0001 for KC01- versus DMSO-treated samples.

FIG. 26 shows the effect of KC01 and KC02 on secreted cytokines from ABHD12^{-/-} macrophages, in particular, the concentration of secreted cytokines from thioglycollate-elicited peritoneal mouse macrophages treated with inhibitors (KC01 or KC02, 1 μM) or DMSO for 4 h, followed by treatment with vehicle (PBS) or LPS (5 μg/mL) for 7 h. Data represent mean values±s.e.m. for four biological replicates. Student's t-test: * p<0.05, *** p<0.0001 for KC01- versus DMSO-treated samples.

FIG. 27 shows the cellular lyso-PS content in ABHD12/mouse peritoneal macrophages. The mouse thioglycollate-elicited peritoneal macrophages derived from ABHD12^{-/-} mice were treated with vehicle (PBS, a) or LPS (5 μg/mL, b) for 7 hours. Thereafter changes in the cellular lyso-PS levels were measured by MRM methods. Details for the sample preparation and analysis can be found herein. The cellular lyso-PS levels for both vehicle (a) and LPS-stimulated (b) macrophages were comparable to those observed for the ABHD12^{+/+} macrophages (FIG. 20d). Each group had four independent biological replicates and the bars represent data as mean±s.e.m.

FIG. 28 shows the concentrations of secreted TNF-α (a) and IL-6 (b) from mouse peritoneal macrophages following treatment with DMSO or varying concentrations of C18:0 free fatty acid (FFA) or C18:0 lyso-PS (4 h treatment period). Treatments were performed in serum-free media. Data represent mean values±s.e.m. N=8 per group. Student's t-test: # p<0.0005 for C18:0 lyso-PS-versus DMSO-treated samples.

FIG. 29 shows the construct design and PCR genotyping for ABHD16A^{-/-} mice. (a) Schematic representation of the construct design for generation of the ABHD16A^{-/-} mice. The construct targeting the Abhd16A allele was generated by Wellcome Trust Sanger Institute (http://www.mousephenotype.org/martsearch_ikmc_project/martsearch/ikmc_project/768 29). Upper diagram shows expected PCR products for genotyping, where primers are located to generate 536 and 198 bp products for ABHD16A^{+/+} and ABHD16A^{-/-} genotypes, respectively. (b) A representative gel for the genotyping of ABHD16A mice is shown. Mouse tail genomic DNA was analyzed by standard PCR-based genotyping protocols. Homozygous ABHD16A^{+/+} mice show a single band of 536 bp, while homozygous ABHD16A^{-/-} mice show a single band at 198 bp. These PCR fragment sizes match those expected for the construct design.

FIG. 30 shows the reduced size of ABHD16A^{-/-} mice. (a, b) Pictures of ABHD16A^{+/+}, ^{+/-}, and ^{-/-} littermate male mice at 10 weeks (a) and 1-day (b) of age showing smaller size of ABHD16^{-/-} animals. (c, d) Body weights of the ABHD16A^{+/+} and ^{-/-} male (c) and female (d) mice recorded from weaning (four weeks) until 10 weeks of age. Data represent mean±s.e.m. N=4 per group.

FIG. 31 shows data related to the generation and characterization of ABHD16A^{-/-} mice. (a, b) Confirmation of absence of ABHD16A mRNA and protein activity in brain tissue from ABHD16A^{-/-} mice using RT-PCR (a) and ABPP (b) analyses, respectively. ABPP gel represents treatment of cerebellar membrane proteomes ABHD16A^{+/+}, ^{+/-}, and ^{-/-} mice with FP-rhodamine (2 μM, 30 min, 37° C.). (c) PS lipase activity of brain membrane lysates from ABHD16A^{+/+}, ^{+/-}, and ^{-/-} mice. Data represent mean values±s.e.m. for three biological replicates. Student's t-test: ** p<0.0005, *** p<0.0001 for ABHD16^{-/-} versus ABHD16A^{+/+} groups. (d) Concentrations of lyso-PS from brain tissue of ABHD16A^{+/+}, ^{+/-}, and ^{-/-} mice. Data represent mean values±s.e.m. for four biological replicates. Student's t-test: * p<0.05, ** p<0.005, for ABHD16^{-/-} versus ABHD16A^{+/+} groups. (e-g) Concentrations of cellular (e) and secreted (f) lyso-PS and TNF- (g) from thioglycollate-elicited peritoneal macrophages derived from ABHD16A^{+/+}, ^{+/-}, and ^{-/-} mice after treatment with vehicle (PBS) or LPS (5 μg/mL) for 7 h. Data represent mean values±s.e.m. for four biological replicates Student's t-test: ** p<0.0005, *** p<0.0001, ABHD16^{-/-} versus ABHD16A^{+/+} groups.

FIG. 32 shows the serine hydrolase activity profile for whole brain membrane proteomes from ABHD16A^{+/+}, ^{+/-}, and ^{-/-} mice. ABPP analysis of whole brain membrane proteomes from ABHD16A^{+/+}, ^{+/-}, and ^{-/-} mice treated with (a) FP-rhodamine (2 μM, 30 min, 37° C.) or (b) WHP01 (0.5 μM, 30 min, 37° C.) confirms complete and selective loss of ABHD16A in ABHD16A^{-/-} mice.

FIG. 33 shows quantitative MS-based ABPP confirms loss of ABHD16A in brain tissue from ABHD16A^{-/-} mice. Serine hydrolase activities were measured by an ABPP-quantitative MS-based proteomic method involving reductive dimethylation (ReDiMe) of tryptic peptides with heavy and light formaldehyde. See Example 28 for more details. Data represent average median ratios±s.e.m. for all quantified tryptic peptides per enzyme from two biological replicates.

FIG. 34 shows the analysis of spinal cord tissue of ABHD16A^{-/-} mice. ABPP analysis of the membrane fraction of spinal cord tissue from ABHD16A^{+/+}, ABHD16A^{+/-} and ABHD16A^{-/-} mice treated with (a) FP-rhodamine (2 μM, 30 min, 37° C.) or (b) WHP01 (0.5 μM, 30 min, 37° C.) confirms complete and selective loss of ABHD16A in spinal cord tissue from ABHD16A^{-/-} mice. (c) RT-PCR analysis of spinal cord tissue from ABHD16A^{+/+} and ABHD16^{-/-} mice confirms the absence of ABHD16A mRNA in the ABHD16A^{-/-} spinal cords. (d) PS lipase activity of spinal cord membrane proteomes from ABHD16A^{+/+}, ABHD16A^{+/-} and ABHD16A^{-/-} mice measured using a C18:0/C18:2 PS substrate (100 μM) as described in Example 32. Data represent mean±s.e.m. N=3 per group. Student's t-test: * p<0.05, ** p<0.005 versus ABHD16A^{+/+} groups (e) Concentrations of lyso-PS lipids from spinal cords of ABHD16A^{++,} ABHD16A^{+/-} and ABHD16A^{-/-} mice. Data represent mean±s.e.m. N=4 per group. Student's t-test: * p<0.05, ** p<0.005 for ABHD16A^{-/-} versus ABHD16A^{+/+} groups.

FIG. 35 shows analysis of thioglycollate-elicited peritoneal macrophages from ABHD16A^{-/-} mice. (a) ABPP gel showing the loss of ABHD16A activity in the membrane fraction of macrophages from ABHD16A^{-/-} mice (vehicle and LPS treatment). Membrane lysates from macrophages of ABHD16A^{+/+} and ABHD16A^{-/-} mice were treated with FP-rhodamine (2 μM, 30 min, 37° C.) or WHP01 (0.5 μM, 30 min, 37° C.). Loss of ABHD16A was confirmed by WHP01, whereas FP-rhodamine could not resolve ABHD16A signals relative to other macrophage serine hydrolase activities. (b) PS lipase activity of peritoneal macrophages from ABHD16A^{+/+}, ^{+/-}, and ^{-/-} mice. Thioglycollate-elicited peritoneal macrophages were harvested from ABHD16A^{+/+}, ABHD16A^{+/-} and ABHD16A^{-/-} mice and treated with vehicle (PBS) or LPS (5 μg/mL) for 7 h. Macrophage membrane fractions were then isolated and analyzed for PS lipase activity using a C18:0/C18:2 PS substrate (100 μM) as described in Example 32. Note that ABHD16A deletion caused a significant reduction in macrophage PS lipase activity, and LPS treatment induced a significant increase in PS lipase activity in ABHD16A^{+/+} and ^{+/-}, but not ^{-/-} macrophages. Data represent mean±s.e.m. N=3 per group. Student's t-test: * p<0.05, ** p<0.005 for LPS- versus PBS-treated groups and S** p<0.001 for ABHD16A^{-/-} versus ABHD16A^{+/+} groups.

FIG. 36 shows secreted cytokine profiles of peritoneal macrophages from ABHD16A^{-/-} mice. Shown are concentration of secreted cytokines IL-6 (a) and IL-1β (b) from thioglycollate-elicited peritoneal mouse macrophages from ABHD16A^{+/+}, +/− and ^{-/-} mice treated with vehicle (PBS) or LPS (5 μg/mL) for 7 h. Data represent mean±s.e.m. for four biological replicates. Student's t-test: ** p<0.005, *** p<0.0001 for ABHD16^{-/-} versus ABHD16^{+/+} groups.

FIG. 37 shows inhibition of PS lipase activity in mouse brain lysates from ABHD16A^{+/+} and ABHD16A^{-/-} mice. (a) PS lipase activity of mouse brain membrane lysates from ABHD16A^{+/+} and ABHD16A^{-/-} mice treated with KC01 or KC02 (1 μM, 30 min, 37° C.) and thereafter assayed using a C18:0/C18:2 PS substrate (100 μM) is shown. Data represent mean±s.e.m. for three biological replicates. Student's t-test: ** p<0.005 for KC01-treated versus DMSO-treated brain membrane lysates. Concentration-dependent inhibition of the PS lipase activities of brain membrane lysates from ABHD12^{+/+} (b) and ABHD12^{-/-} (c) mice is shown. Indicated concentrations of KC01 and KC02 were incubated with brain membrane lysates (30 min, 37° C.) and thereafter assayed using a C18:0/C18:2 PS substrate (100 μM). Data represent mean±s.e.m. for three biological replicates.

FIG. 38 shows activity dependent labeling of ABHD16A by WHP01. (a) Structure of WHP01, a fluorescent probe for ABHD16A. (b, c) ABPP gel of HEK293T cell lysates transfected with mock, wildtype (WT)-ABHD16A (FLAG-tag) or a catalytic serine S355A-ABHD16A mutant (FLAG-tag) treated with (b) FP-rhodamine (2 μM, 30 min, 37° C.) or (c) WHP01 (0.2 μM, 30 min, 37° C.). (d) Western blot analysis (anti-FLAG) of the mock-, WT-ABHD16A-, and S355A-ABHD16A-transfected HEK293T lysates confirming the expression of WT- and S355A-ABHD16A in HEK293T cells. (e) Concentration-dependent labeling of endogenous ABHD16A in mouse brain membrane lysates by FP-rhodamine and WHP01 (30 min, 37° C.).

FIG. 39 shows the effect of KC01 and KC02 on the secreted lyso-PS and cytokines from ABHD16A^{-/-} macrophages. Concentration of secreted lyso-PS (a) and cytokines IL-6 (b) and TNF-α (c) from thioglycollate-elicited peritoneal mouse macrophages from ABHD16A^{-/-} mice treated with inhibitor (KC01 or KC02, 1 μM, 4 h) followed by vehicle (PBS) or LPS (5 μg/mL) for 7 h is shown. Data represent mean±s.e.m. for four biological replicates. KC01 and KC02 have no effect on lyso-PS or cytokine secretion. Other secreted and cellular lipids in ABHD16A^{-/-} macrophages were also unchanged by KC01 or KC02 treatment (data not shown).

### DETAILED DESCRIPTION OF THE INVENTION

This disclosure is directed, at least in part, to ABHD16A inhibitors and uses thereof.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl, and each R^{f} is independently alkyl, fluoroalkyl, carbocyclyl, carbocyclylalkyl, aryl, aralkyl, heterocyclyl, heterocyclylalkyl, heteroaryl or heteroarylalkyl.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl, and each R^{f} is independently alkyl, fluoroalkyl, carbocyclyl, carbocyclylalkyl, aryl, aralkyl, heterocyclyl, heterocyclylalkyl, heteroaryl or heteroarylalkyl.

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl, and each R^{f} is independently alkyl, fluoroalkyl, carbocyclyl, carbocyclylalkyl, aryl, aralkyl, heterocyclyl, heterocyclylalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₅ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl, and each R^{f} is independently alkyl, fluoroalkyl, carbocyclyl, carbocyclylalkyl, aryl, aralkyl, heterocyclyl, heterocyclylalkyl, heteroaryl or heteroarylalkyl.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aryloxy” refers to a radical bonded through an oxygen atom of the formula —O-aryl, where aryl is as defined above.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Carbocyclyl” refers to a stable non-aromatic monocyclic or polycyclic hydrocarbon radical consisting solely of carbon and hydrogen atoms, which may include fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl may be saturated, (i.e., containing single C—C bonds only) or unsaturated (i.e., containing one or more double bonds or triple bonds.) A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. In certain embodiments, a cycloalkyl comprises three to eight carbon atoms (e.g., C₃-C₈ cycloalkyl). In other embodiments, a cycloalkyl comprises three to seven carbon atoms (e.g., C₃-C₇ cycloalkyl). In other embodiments, a cycloalkyl comprises three to six carbon atoms (e.g., C₃-C₆ cycloalkyl). In other embodiments, a cycloalkyl comprises three to five carbon atoms (e.g., C₃-C₅ cycloalkyl). In other embodiments, a cycloalkyl comprises three to four carbon atoms (e.g., C₃-C₄ cycloalkyl). An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. The alkyl part of the fluoroalkyl radical may be optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which may include fused or bridged ring systems. The heteroatoms in the heterocyclyl radical may be optionally oxidized. One or more nitrogen atoms, if present, are optionally quaternized. The heterocyclyl radical is partially or fully saturated. The heterocyclyl may be attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7, 8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroaryloxy” refers to radical bonded through an oxygen atom of the formula —O— heteroaryl, where heteroaryl is as defined above.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain.

The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein may contain one or more asymmetric centers and may thus give rise to enantiomers, diastereomers, and other stereoisomeric forms that may be defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein may, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

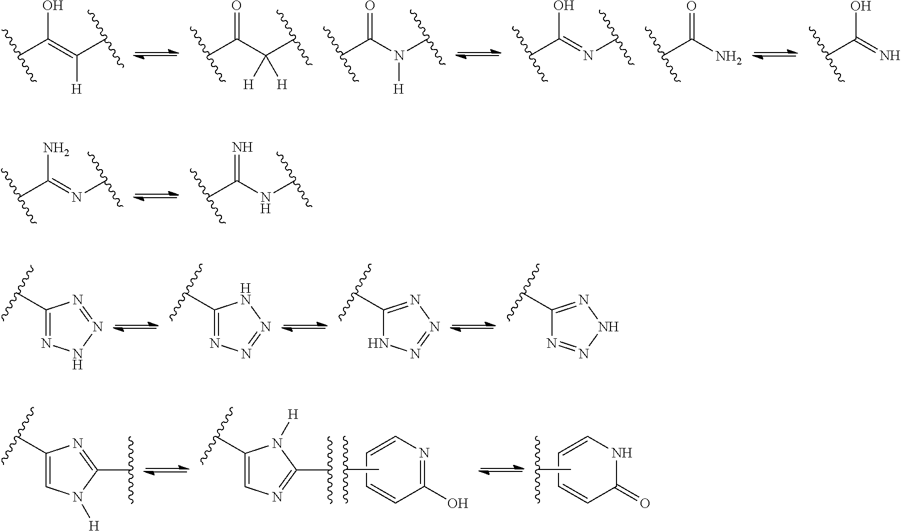

“Optional” or “optionally” means that a subsequently described event or circumstance may or may not occur and that the description includes instances when the event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable salt” includes both acid and base addition salts. A pharmaceutically acceptable salt of any one of the lactone compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates. See, e.g., Berge S. M. et al., “Pharmaceutical Salts,” Journal of Pharmaceutical Science, 66:1-19 (1997). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, “treatment” or “treating” or “palliating” or “ameliorating” are used interchangeably herein. These terms refers to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

“Prodrug” is meant to indicate a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism. See, e.g., Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of alcohol or amine functional groups in the active compounds and the like.

### Compounds

Lactone compounds are described herein. In some embodiments, the lactone compounds disclosed herein are inhibitors of ABHD16A. These compounds, and compositions comprising these compounds, are useful for the treatment of PHARC and/or neuroinflammatory disease.

One embodiment provides a compound of Formula (I):

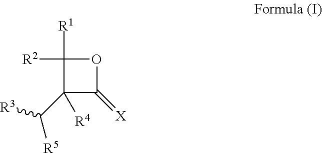

wherein:
X is O or CH₂;
R¹ is alkyl or -alkylene-(optionally substituted aryl);
R² is H;
R³ is a optionally substituted aryl, optionally substituted aralkyl, or optionally substituted alkyl; and
R⁴ and R⁵ are both H; or R⁴ and R⁵ together form a direct bond to provide a double bond;
or a solvate, hydrate, tautomer, N-oxide, stereoisomer, or pharmaceutically acceptable salt thereof.

In another embodiment is a compound of Formula (I), wherein X is O.

In another embodiment is a compound of Formula (I), wherein X is CH₂.

In another embodiment is a compound of Formula (I), wherein R⁴ and R⁵ are both H.

In another embodiment is a compound of Formula (I), wherein R⁴ and R⁵ together form a direct bond to provide a double bond.

In another embodiment is a compound of Formula (I), wherein R³ comprises a fluorophore.

Another embodiment provides a compound of Formula (II):

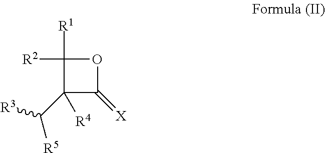

wherein:
X is O or CH₂;
R¹ is alkyl or -alkylene-(optionally substituted aryl);
R² is H;
R³ is optionally substituted aryl, —(CH₂)ₙ—CH₃, —(CH₂)ₙ-(optionally substituted aryl), —(CH₂)ₙ—C(O)NR⁶R⁷, or —(CH₂)ₙ—SO₂NR⁶R⁷;
R⁴ and R⁵ are both H; or R⁴ and R⁵ together form a direct bond to provide a double bond;
R⁶ is H or alkyl;
R⁷ is H or alkyl; and
n is 1, 2, 3, 4, 5, or 6;
or a solvate, hydrate, tautomer, N-oxide, stereoisomer, or pharmaceutically acceptable salt thereof.

In some embodiments of a compound of Formula (II), R¹ is alkyl or -alkylene-(optionally substituted phenyl). In some embodiments of a compound of Formula (II), R¹ is alkyl. In some embodiments of a compound of Formula (II), R¹ is-alkylene-(optionally substituted aryl). In some embodiments of a compound of Formula (II), R³ is optionally substituted phenyl, —(CH₂)ₙ—CH₃, —(CH₂)ₙ-(optionally substituted phenyl), —(CH₂)ₙ—C(O)NR⁶R⁷, or —(CH₂)ₙ—SO₂NR⁶R⁷. In some embodiments of a compound of Formula (II), R³ is optionally substituted aryl. In some embodiments of a compound of Formula (II), R³ is —(CH₂)ₙ—CH₃. In some embodiments of a compound of Formula (II), R³ is —(CH₂)ₙ-(optionally substituted aryl). In some embodiments of a compound of Formula (II), R³ is —(CH₂)ₙ—C(O)NR⁶R⁷. In some embodiments of a compound of Formula (II), R³ is —(CH₂)ₙ—SO₂NR⁶R⁷. In some embodiments of a compound of Formula (II), R³ is —(CH₂)ₙ—C(O)NR⁶R⁷ or —(CH₂)ₙ—SO₂NR⁶R⁷. In some embodiments of a compound of Formula (II), R⁶ and R⁷ are both H. In some embodiments of a compound of Formula (II), R⁴ and R⁵ are both H. In some embodiments of a compound of Formula (II), R⁴ and R⁵ together form a direct bond to provide a double bond. In some embodiments of a compound of Formula (II), X is O. In some embodiments of a compound of Formula (II), X is CH₂. In some embodiments of a compound of Formula (II), R⁴ and R⁵ together form a direct bond to provide a double bond; and X is O. In some embodiments of a compound of Formula (II), R⁴ and R⁵ together form a direct bond to provide a double bond; and X is CH₂.

Another embodiment provides a compound of Formula (III):

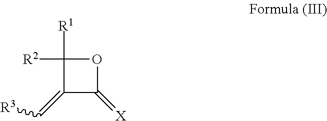

wherein:
X is O or CH₂; R is alkyl or -alkylene-(optionally substituted aryl);
R² is H;
R³ is optionally substituted aryl, —(CH₂)ₙ—CH₃, —(CH₂)ₙ-(optionally substituted aryl), —(CH₂)ₙ—C(O)NR⁶R⁷, or —(CH₂)ₙ—SO₂NR⁶R⁷;
R⁶ is H or alkyl;
R⁷ is H or alkyl; and
n is 1, 2, 3, 4, 5, 6, 7, 8, 9, 10, 11, 12, 13, 14, 15, 16, 17, 18, 19, or 20;
or a solvate, hydrate, tautomer, N-oxide, stereoisomer, or pharmaceutically acceptable salt thereof.

In some embodiments of a compound of Formula (III), R¹ is alkyl or -alkylene-(optionally substituted phenyl). In some embodiments of a compound of Formula (III), R¹ is alkyl. In some embodiments of a compound of Formula (III), R¹ is -alkylene-(optionally substituted aryl). In some embodiments of a compound of Formula (III), R³ is optionally substituted phenyl, —(CH₂)ₙ—CH₃, —(CH₂)ₙ-(optionally substituted phenyl), —(CH₂)ₙ—C(O)NR⁶R⁷, or —(CH₂)ₙ—SO₂NR⁶R⁷. In some embodiments of a compound of Formula (III), R³ is optionally substituted aryl. In some embodiments of a compound of Formula (III), R³ is —(CH₂)ₙ—CH₃. In some embodiments of a compound of Formula (III), R³ is —(CH₂)ₙ-(optionally substituted aryl). In some embodiments of a compound of Formula (III), R³ is —(CH₂)ₙ—C(O)NR⁶R⁷. In some embodiments of a compound of Formula (III), R³ is —(CH₂)ₙ—SO₂NR⁶R⁷. In some embodiments of a compound of Formula (III), R³ is —(CH₂)ₙ—C(O)NR⁶R⁷ or —(CH₂)ₙ—SO₂NR⁶R⁷. In some embodiments of a compound of Formula (III), R⁶ and R⁷ are both H. In some embodiments of a compound of Formula (III), X is O. In some embodiments of a compound of Formula (III), X is CH₂. In some embodiments of a compound of Formula (III), n is 1, 2, 3, 4, 5, or 6.

Another embodiment provides a compound of Formula (IV):

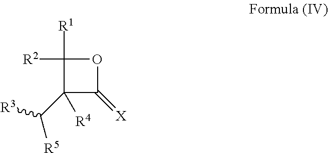

wherein:
X is O or CH₂;
R¹ is alkyl or -alkylene-(phenyl);
R² is H;
R³ is phenyl, —(CH₂)ₙ—CH₃, —(CH₂)ₙ-(phenyl), —(CH₂)ₙ—C(O)NH₂, or —(CH₂)ₙ—SO₂NH₂;
R⁴ and R⁵ are both H; or R⁴ and R⁵ together form a direct bond to provide a double bond; and
n is 1, 2, 3, 4, 5, or 6;
or a solvate, hydrate, tautomer, N-oxide, stereoisomer, or pharmaceutically acceptable salt thereof.

In some embodiments of a compound of Formula (IV), R¹ is alkyl. In some embodiments of a compound of Formula (IV), R¹ is -alkylene-(phenyl). In some embodiments of a compound of Formula (IV), R³ is phenyl. In some embodiments of a compound of Formula (IV), R³ is —(CH₂)ₙ—CH₃. In some embodiments of a compound of Formula (IV), R³ is —(CH₂)ₙ-(phenyl). In some embodiments of a compound of Formula (IV), R³ is —(CH₂)ₙ—C(O)NH₂. In some embodiments of a compound of Formula (IV), R³ is —(CH₂)ₙ—SO₂NH₂. In some embodiments of a compound of Formula (IV), R³ is —(CH₂)ₙ—C(O)NH₂ or —(CH₂)ₙ—SO₂NH₂. In some embodiments of a compound of Formula (IV), R⁴ and R⁵ are both H. In some embodiments of a compound of Formula (IV), R⁴ and R⁵ together form a direct bond to provide a double bond. In some embodiments of a compound of Formula (IV), X is O. In some embodiments of a compound of Formula (IV), X is CH₂. In some embodiments of a compound of Formula (IV), R⁴ and R⁵ together form a direct bond to provide a double bond and X is O. In some embodiments of a compound of Formula (IV), R⁴ and R⁵ together form a direct bond to provide a double bond and X is CH₂.

Another embodiment provides a compound of Formula (V):

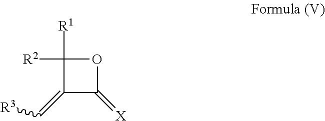

wherein:
X is O or CH₂;
R¹ is alkyl or -alkylene-(phenyl);
R² is H;
R³ is phenyl, —(CH₂)ₙ—CH₃, —(CH₂)ₙ-(phenyl), —(CH₂)ₙ—C(O)NH₂, or —(CH₂)ₙ—SO₂NH₂;
n is 1, 2, 3, 4, 5, 6, 7, 8, 9, 10, 11, 12, 13, 14, 15, 16, 17, 18, 19, or 20;
or a solvate, hydrate, tautomer, N-oxide, stereoisomer, or pharmaceutically acceptable salt thereof

In some embodiments of a compound of Formula (V), R¹ is alkyl. In some embodiments of a compound of Formula (V), R¹ is -alkylene-(phenyl). In some embodiments of a compound of Formula (V), R³ is phenyl. In some embodiments of a compound of Formula (V), R³ is —(CH₂)ₙ—CH₃. In some embodiments of a compound of Formula (V), R³ is —(CH₂)ₙ-(phenyl).

In some embodiments of a compound of Formula (V), R³ is —(CH₂)ₙ—C(O)NH₂. In some embodiments of a compound of Formula (V), R³ is —(CH₂)ₙ—SO₂NH₂. In some embodiments of a compound of Formula (V), R³ is —(CH₂)ₙ—C(O)NH₂ or —(CH₂)ₙ—SO₂NH₂. In some embodiments of a compound of Formula (V), X is O. In some embodiments of a compound of Formula (V), X is CH₂. In some embodiments of a compound of Formula (V), n is 1, 2, 3, 4, 5, or 6. In some embodiments of a compound of Formula (V), X is CH₂; n is 1, 2, 3, 4, 5, or 6; and R³ is —(CH₂)ₙ—C(O)NH₂, or —(CH₂)ₙ—SO₂NH₂. In some embodiments of a compound of Formula (V), X is O; n is 1, 2, 3, 4, 5, or 6; and R³ is —(CH₂)ₙ—C(O)NH₂, or —(CH₂)ₙ—SO₂NH₂.

Another embodiment provides a compound of Formula (VI):

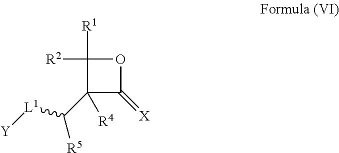

wherein:
X is O or CH₂;
R¹ is alkyl or -alkylene-(optionally substituted aryl);
R² is H;
R⁴ and R⁵ are both H; or R⁴ and R⁵ together form a direct bond to provide a double bond;
L¹ is a linker; and
Y is a fluorophore;
or a solvate, hydrate, tautomer, N-oxide, stereoisomer, or pharmaceutically acceptable salt thereof.

In another embodiment is a compound of Formula (VI), wherein X is O.

In another embodiment is a compound of Formula (VI), wherein X is CH₂.

In another embodiment is a compound of Formula (VI), wherein R⁴ and R⁵ are both H.

In another embodiment is a compound of Formula (VI), wherein R⁴ and R⁵ together form a direct bond to provide a double bond.

In another embodiment is a compound of Formula (VI), wherein X is O and R⁴ and R⁵ together form a direct bond to provide a double bond.

In another embodiment is a compound of Formula (VI), wherein X is O and R⁴ and R⁵ are both H.

It is envisioned that the fluorophore may be, but is not limited to, rhodamine, coumarin, cyanine, xanthene, polymethine, pyrene, dipyrromethene borondifluoride, napthalimide, a phycobiliprotein, peridinium chlorophyll proteins, conjugates thereof, derivatives thereof, and combinations thereof. Non-limiting examples of fluorophores include fluorescein, 6-FAM, rhodamine, Texas Red, California Red, iFluor594, carboxytetramethylrhodamine, tetramethylrhodamine, a carboxyrhodamine, carboxyrhodamine 6F, carboxyrhodol, carboxyrhodamine 110, Cascade Blue, Cascade Yellow, coumarin, Cy2®, Cy3®, Cy3.5®, Cy5®, Cy5.5®, Cy7®, Cy-Chrome, DyLight 350, DyLight 405, DyLight 488, DyLight 549, DyLight 594, DyLight 633, DyLight 649, DyLight 680, DyLight 750, DyLight 800, phycoerythrin, PerCP (peridinin chlorophyll-a Protein), PerCP-Cy5.5, JOE (6-carboxy-4′,5′-dichloro-2′,7′-dimethoxyfluorescein), NED, ROX (5-(and-6-)-carboxy-X-rhodamine), HEX, Lucifer Yellow, Marina Blue, Oregon Green 488, Oregon Green 500, Oregon Green 514, Alexa Fluor® 350, Alex Fluor® 430, Alexa Fluor® 488, Alexa Fluor® 532, Alexa Fluor® 546, Alexa Fluor® 568, Alexa Fluor® 594, Alexa Fluor® 633, Alexa Fluor® 647, Alexa Fluor® 660, Alexa Fluor® 680, 7-amino-4-methylcoumarin-3-acetic acid, BODIPY® FL, BODIPY® FL-Br₂, BODIPY® 530/550, BODIPY® 558/568, BODIPY® 630/650, BODIPY® 650/665, BODIPY® R6G, BODIPY® TMR, BODIPY® TR, conjugates thereof, derivatives thereof, analogs thereof, and combinations thereof.

In some embodiments of a compound of Formula (VI), L¹ is a bond.

In some embodiments of a compound of Formula (VI), L¹ comprises one or more amides, alkylenes, alkenes, alkynes, ethers, thioethers, amines, sulfonamides, ureas, carbamates, or esters, or combinations thereof.

Another embodiment provides a compound selected from:

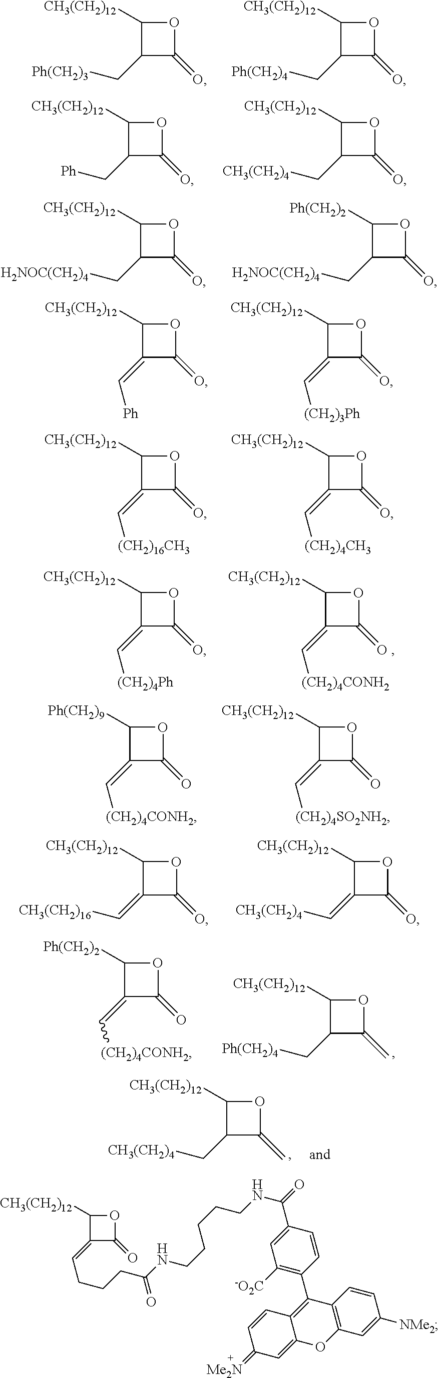

or a solvate, hydrate, tautomer, N-oxide, stereoisomer, or a pharmaceutically acceptable salt thereof.

Further embodiments provided herein include combinations of one or more of the particular embodiments set forth above.

In some embodiments, the compound disclosed herein has the structure provided in Table 1.

TABLE 1

| Chemical | | |
|---|---|---|
| Synthesis | | |
| Example | Structure | Name |
| 1a | 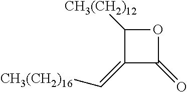 | (E)-3-octadecylidene-4-tridecyloxetan-2-one |
| 1b | 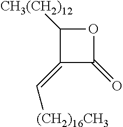 | (Z)-3-octadecylidene-4-tridecyloxetan-2-one |
| 2a | 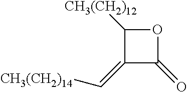 | (E)-3-hexylidene-4-tridecyloxetan-2-one |
| 2b | 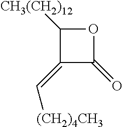 | (Z)-3-hexylidene-4-tridecyloxetan-2-one |
| 3 | 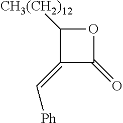 | (Z)-3-benzylidene-4-tridecyloxetan-2-one |
| 4 | 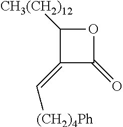 | (Z)-3-(5-phenylpentylidene)-4-tridecyloxetan-2-one |
| 5 | 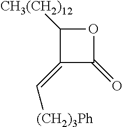 | (Z)-3-(4-phenylbutylidene)-4-tridecyloxetan-2-one |
| 6a | 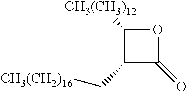 | (cis)-3-octadecyl-4-tridecyloxetan-2-one |
| 6b | 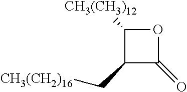 | (trans)-3-octadecyl-4-tridecyloxetan-2-one |
| 7a | 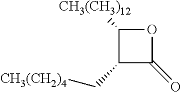 | (cis)-3-3-hexyl-4-tridecyloxetan-2-one |
| 7b | 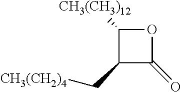 | (trans)-3-3-hexyl-4-tridecyloxetan-2-one |
| 8a | 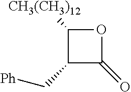 | (cis)-3-benzyl-4-tridecyloxetan-2-one |
| 8b | 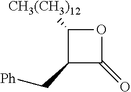 | (trans)-3-benzyl-4-tridecyloxetan-2-one |
| 9a | 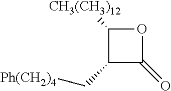 | (cis)-3-(5-phenylpentyl)-4-tridecyloxetan-2-one |
| 9b | 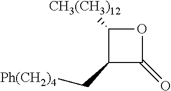 | (trans)-3-(5-phenylpentyl)-4-tridecyloxetan-2-one |
| 10a | 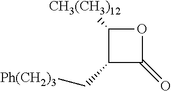 | (cis)-3-(4-phenylbutyl)-4-tridecyloxetan-2-one |
| 10b | 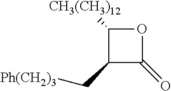 | (trans)-3-(4-phenylbutyl)-4-tridecyloxetan-2-one |
| 11 | 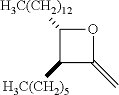 | (trans)-3-hexyl-2-methylene-4-tridecyloxetane |
| 12 | 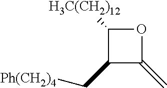 | (trans)-2-methylene-3-(5-phenylpentyl)-4- tridecyloxetane |
| 13b | 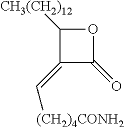 | (Z)-6-(2-oxo-4-tridecyloxetan-3-ylidene)hexanamide |
| 14 | 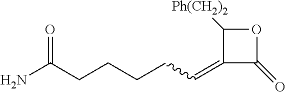 | (E/Z)-6-(2-oxo-4-(2-phenethyl)oxetan-3-ylidene) hexanamide |
| 15 | 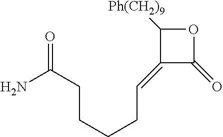 | (Z)-6-(2-oxo-4-(9-phenylnonyl)oxetane-3-ylidene) hexanamide |
| 16 | 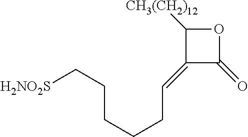 | (Z)-6-(2-Oxo-4-tridecyloxetan-3-ylidene)hexane-1- sulfonamide) |
| 17 | 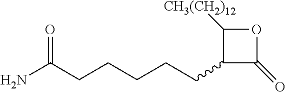 | (cis/trans)-6-(2-oxo-4-tridecyloxetan-3-yl) hexanamide |
| 17a | 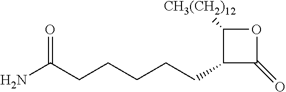 | (cis)-6-(2-oxo-4-tridecyloxetan-3-yl)hexanamide |
| 18 | 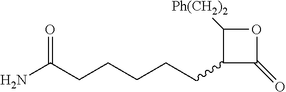 | (cis/trans)-6-(2-oxo-4-(2-phenethyl) oxetan-3-yl)hexanamide |
| 19 | 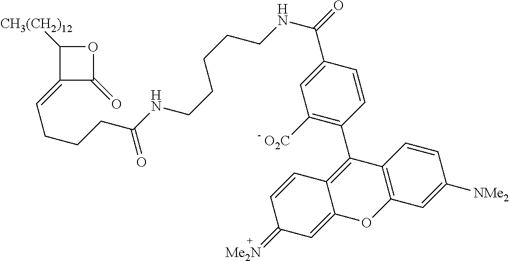 | (Z)-2-(6-(dimethylamino)-3-(dimethyliminio)-3H- xanthen-9-yl)-5-((5-(5-(2-oxo-4-tridecyloxetan- 3-ylidene)pentanamido)pentyl) carbamoyl)benzoate |

### Preparation of the Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, Pa.), Aldrich Chemical (Milwaukee, Wis., including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

Methods known to one of ordinary skill in the art are identified through various reference books and databases. Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the lactone compounds described herein is P. H. Stahl & C. G. Wermuth “Handbook of Pharmaceutical Salts”, Verlag Helvetica Chimica Acta, Zurich, 2002.

The lactone compounds are prepared by the general synthetic routes described below in Schemes 1-4.

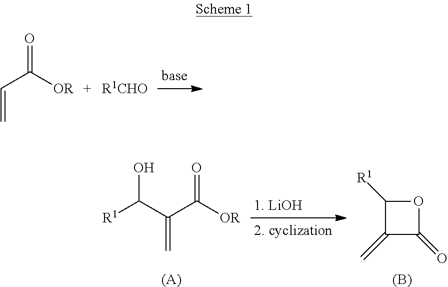

A method for preparing intermediates of formula B is provided in Scheme 1. An aldehyde and acrylic acid ester undergo a Morita-Baylis-Hilman reaction under basic conditions to afford an intermediate of formula A. Saponification followed by cyclization leads to an α-methylene-β-lactone of formula B.

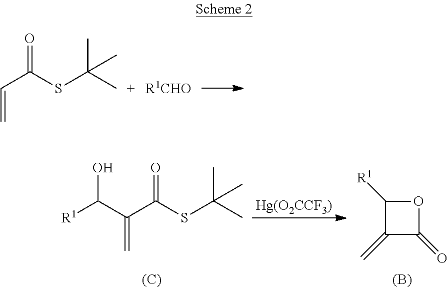

An alternative method for preparing intermediates of formula B is provided in Scheme 2. A Morita-Baylis-Hilman reaction between an aldehyde and thio-tert-butyl acrylate results in a intermediate of formula C. Mercury-mediated cyclization leads to an α-methylene-β-lactone of formula B.

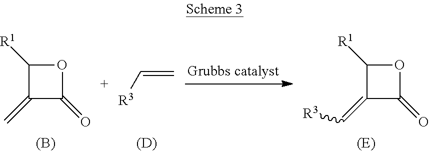

A method of preparing compounds of formula E is provided in Scheme 3. Compounds of formula E are prepared by cross-metathesis between a compound of formula B and an alkene of formula D.

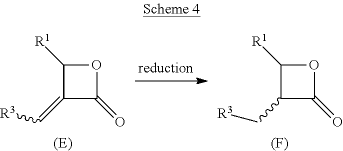

A method of preparing compounds of formula F is provided in Scheme 4. Reduction of the alkene of a compound of formula E results in a compound of formula F. In some embodiments, stereoselective reduction conditions comprise hydrogenation with a palladium catalyst. In some embodiments, stereoselective reduction conditions comprise CoCl₂(PPh)₃ and NaBH₄.

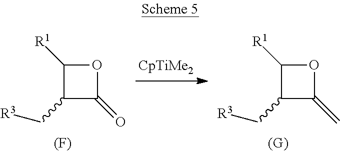

A method of preparing compounds of formula G is provided in Scheme 5. Methylenation of a compound of formula F is performed using a reagent such as Petasis reagent or Tebbe reagent to obtain a compound of formula G.

### Further Forms of Lactone Compounds Disclosed Herein

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the metabolites, pharmaceutically acceptable salts, esters, prodrugs, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, prodrug, solvate, hydrate or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the invention, or by separately reacting a purified compound in its free form with a suitable acid or base, and isolating the salt thus formed.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Prodrugs

In some embodiments, the compounds described herein exist in prodrug form. The invention provides for methods of treating diseases by administering such prodrugs. The invention further provides for methods of treating diseases by administering such prodrugs as pharmaceutical compositions.

In some embodiments, prodrugs include compounds wherein an amino acid residue, or a polypeptide chain of two or more (e. g., two, three or four) amino acid residues is covalently joined through an amide or ester bond to a free amino, hydroxy or carboxylic acid group of compounds of the present invention. The amino acid residues include but are not limited to the 20 naturally occurring amino acids and also includes 4-hydroxyproline, hydroxylysine, demosine, isodemosine, 3-methylhistidine, norvaline, beta-alanine, gamma-aminobutyric acid, citrulline, homocysteine, homoserine, ornithine and methionine sulfone. In other embodiments, prodrugs include compounds wherein a nucleic acid residue, or an oligonucleotide of two or more (e. g., two, three or four) nucleic acid residues is covalently joined to a compound of the present invention.

Pharmaceutically acceptable prodrugs of the compounds described herein also include, but are not limited to, esters, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts and sulfonate esters. Compounds having free amino, amido, hydroxy or carboxylic groups can be converted into prodrugs. For instance, free carboxyl groups can be derivatized as amides or alkyl esters. In certain instances, all of these prodrug moieties incorporate groups including but not limited to ether, amine and carboxylic acid functionalities.

Hydroxy prodrugs include esters, such as though not limited to, acyloxyalkyl (e.g. acyloxymethyl, acyloxyethyl) esters, alkoxycarbonyloxyalkyl esters, alkyl esters, aryl esters, phosphate esters, sulfonate esters, sulfate esters and disulfide containing esters; ethers, amides, carbamates, hemisuccinates, dimethylaminoacetates and phosphoryloxymethyloxycarbonyls, as outlined in Advanced Drug Delivery Reviews 1996, 19, 115.

Amine derived prodrugs include, but are not limited to the following groups and combinations of groups:

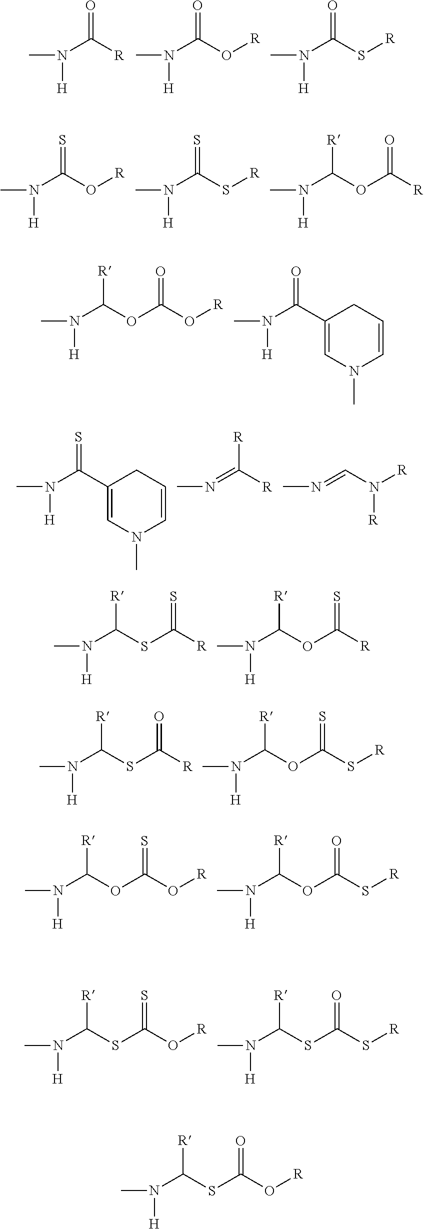

as well as sulfonamides and phosphonamides.

In certain instances, sites on any aromatic ring portions are susceptible to various metabolic reactions, therefore incorporation of appropriate substituents on the aromatic ring structures, can reduce, minimize or eliminate this metabolic pathway.

### Metabolites

In some embodiments, lactone compounds described herein are susceptible to various metabolic reactions. Therefore, in some embodiments, incorporation of appropriate substituents into the structure will reduce, minimize, or eliminate a metabolic pathway. In specific embodiments, the appropriate substituent to decrease or eliminate the susceptibility of an aromatic ring to metabolic reactions is, by way of example only, a halogen, or an alkyl group.

In additional or further embodiments, lactone compounds described herein are metabolized upon administration to an organism in need to produce a metabolite that is then used to produce a desired effect, including a desired therapeutic effect.

### Pharmaceutical Compositions

In certain embodiments, the lactone compound as described herein is administered as a pure chemical. In other embodiments, the lactone compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, Pa. (2005)), the disclosure of which is hereby incorporated herein by reference in its entirety.

Accordingly, provided herein is a pharmaceutical composition comprising at least one lactone compound described herein, or a stereoisomer, pharmaceutically acceptable salt, hydrate, solvate, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

One embodiment provides a pharmaceutical composition comprising a pharmaceutically acceptable carrier and a compound of Formula (I), or a pharmaceutically acceptable salt thereof.

One embodiment provides a pharmaceutical composition comprising a pharmaceutically acceptable carrier and a compound of Formula (II), or a pharmaceutically acceptable salt thereof.

One embodiment provides a pharmaceutical composition comprising a pharmaceutically acceptable carrier and a compound of Formula (III), or a pharmaceutically acceptable salt thereof.

One embodiment provides a pharmaceutical composition comprising a pharmaceutically acceptable carrier and a compound of Formula (IV), or a pharmaceutically acceptable salt thereof.

One embodiment provides a pharmaceutical composition comprising a pharmaceutically acceptable carrier and a compound of Formula (V), or a pharmaceutically acceptable salt thereof.

Another embodiment provides a pharmaceutical composition consisting essentially of a pharmaceutically acceptable carrier and a compound of Formula (I), or a pharmaceutically acceptable salt thereof. Another embodiment provides a pharmaceutical composition consisting essentially of a pharmaceutically acceptable carrier and a compound of Formula (II), or a pharmaceutically acceptable salt thereof. Another embodiment provides a pharmaceutical composition consisting essentially of a pharmaceutically acceptable carrier and a compound of Formula (III), or a pharmaceutically acceptable salt thereof. Another embodiment provides a pharmaceutical composition consisting essentially of a pharmaceutically acceptable carrier and a compound of Formula (IV), or a pharmaceutically acceptable salt thereof. Another embodiment provides a pharmaceutical composition consisting essentially of a pharmaceutically acceptable carrier and a compound of Formula (V), or a pharmaceutically acceptable salt thereof.

In certain embodiments, the lactone compound as described herein is substantially pure, in that it contains less than about 5%, or less than about 1%, or less than about 0.1%, of other organic small molecules, such as contaminating intermediates or by-products that are created, for example, in one or more of the steps of a synthesis method.

These formulations include those suitable for oral, rectal, topical, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

Exemplary pharmaceutical compositions may be used in the form of a pharmaceutical preparation, for example, in solid, semisolid or liquid form, which includes one or more of a disclosed compound, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a disclosed compound or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds disclosed herein may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants

Also contemplated are enteral pharmaceutical formulations including a disclosed compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives of the present disclosure.

The dose of the composition comprising at least one lactone compound as described herein may differ, depending upon the patient's (e.g., human) condition, that is, stage of the disease, general health status, age, and other factors that a person skilled in the medical art will use to determine dose.

Pharmaceutical compositions may be administered in a manner appropriate to the disease to be treated (or prevented) as determined by persons skilled in the medical arts. An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses may generally be determined using experimental models and/or clinical trials. The optimal dose may depend upon the body mass, weight, or blood volume of the patient.

Oral doses can typically range from about 1.0 mg to about 1000 mg, one to four times, or more, per day.

Methods

Disclosed herein are methods of inhibiting ABHD16A in mammalian cells. Contemplated methods, for example, comprise contacting the mammalian cells with a compound described herein. In some embodiments, the compound utilized by one or more of the foregoing methods is one of the generic, subgeneric, or specific compounds described herein, such as a compound of Formula (I), (II), (III), (IV), or (V). In some embodiments, the method is an in vitro method. In other embodiments, the method is an in vivo method. The ability of compounds described herein to inhibit ABHD16A can be evaluated by procedures known in the art and/or described herein. Another aspect of this disclosure provides methods of treating a disease associated with expression or activity of ABHD16A in a patient. For example, provided herein are compounds that may be selective in inhibiting ABHD16A as compared to inhibition of other serine hydrolases e.g., FAAH, e.g., 10, 100, 1000 or more fold inhibition of ABHD16A over FAAH.

Also contemplated herein are methods of treating a neuroinflammatory disease or neuroinflammation associated with a neurodegenerative disease. Non-limiting examples include Alzheimer's disease, Parkinson's disease, and multiple sclerosis. Contemplated methods, for example, comprise contacting the mammalian cells with a compound described herein. In some embodiments, the compound utilized by one or more of the foregoing methods is one of the generic, subgeneric, or specific compounds described herein, such as a compound of Formula (I), (II), (III), (IV), or (V). Contemplated methods include administering a pharmaceutically effective amount of a disclosed compound.

In an embodiment, provided herein are methods of reducing cellular and/or secreted levels of pro-inflammatory lysophosphatidylserine lipids in mammalian cells. In some embodiments, the method comprises contacting the mammalian cells with an ABHD16A inhibitor. In some embodiments, the method comprises contacting the mammalian cells with a compound disclosed herein. In some embodiments, the method is an in vitro method. In other embodiments, the method is an in vivo method

In certain embodiments, a disclosed compound utilized by one or more of the foregoing methods is one of the generic, subgeneric, or specific compounds described herein, such as a compound of Formula (I), (II), (III), (IV), or (V).

Disclosed compounds may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician. For treating clinical conditions and diseases noted above, a contemplated compound disclosed herein may be administered orally, subcutaneously, topically, parenterally, by inhalation spray or rectally in dosage unit formulations containing conventional non-toxic pharmaceutically acceptable carriers, adjuvants and vehicles. Parenteral administration may include subcutaneous injections, intravenous or intramuscular injections or infusion techniques.

Also contemplated herein are combination therapies, for example, co-administering a disclosed compound and an additional active agent, as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents.

Administration of these therapeutic agents in combination typically is carried out over a defined time period (usually weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner.

Substantially simultaneous administration can be accomplished, for example, by administering to the subject a single formulation or composition, (e.g., a tablet or capsule having a fixed ratio of each therapeutic agent or in multiple, single formulations (e.g., capsules) for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection.

Combination therapy also can embrace the administration of the therapeutic agents as described above in further combination with other biologically active ingredients and non-drug therapies. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The components of the combination may be administered to a patient simultaneously or sequentially. It will be appreciated that the components may be present in the same pharmaceutically acceptable carrier and, therefore, are administered simultaneously. Alternatively, the active ingredients may be present in separate pharmaceutical carriers, such as, conventional oral dosage forms, that can be administered either simultaneously or sequentially.

Other embodiments and uses will be apparent to one skilled in the art in light of the present disclosures. The following examples are provided merely as illustrative of various embodiments and shall not be construed to limit the invention in any way.

### Examples

### List of Abbreviations

As used above, and throughout the description of the invention, the following abbreviations, unless otherwise indicated, shall be understood to have the following meanings:
ACN or MeCN acetonitrile
Bn benzyl
BOC or Boc tert-butyl carbamate
t-Bu tert-butyl
Cy cyclohexyl
DCE dichloroethane (ClCH₂CH₂Cl)
DCM dichloromethane (CH₂Cl₂)
DIPEA or DIEA diisopropylethylamine
DMF dimethylformamide
DMSO dimethylsulfoxide
eq equivalent(s)
Et ethyl
Et₂O diethyl ether
EtOH ethanol
EtOAc ethyl acetate
FFA free fatty acid
GC gas chromatography
HMPA hexamethylphosphoramide
Lyso-PC lysophosphatidylcholine
Lyso-PE lysophosophatidylethanolamine
Lyso-PS lysophosphatidylserine
MAG monoacylglycerol
Me methyl
MeOH methanol
MS mass spectroscopy
NMR nuclear magnetic resonance
PC phosphatidylcholine
PE phosophatidylethanolamine
PS phosphatidylserine
rt room temperature
TBAF tetra-n-butylammonium fluoride
THF tetrahydrofuran
TLC thin layer chromatography

### I. Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized.

Column chromatography and thin layer chromatography (TLC) were performed on silica gel unless otherwise noted. NMR spectra were obtained on a Bruker Avance DRX-400 (400 MHz ¹H, 100 MHz ¹³C), Bruker Avance (500 MHz ¹H, 125 MHz ¹³C), or Bruker Avance (300 MHz ¹H, 75 MHz ¹³C) spectrometer. NMR spectra are given in ppm (δ) and coupling constants, J are reported in Hertz. For proton spectra the solvent peak was used as the reference peak. IR spectra were recorded on a Brucker FT-IR spectrometer. GC/MS spectra were obtained on a gas chromatograph equipped with a HP-1 methyl siloxane column and detected on a low-resolution 5970 series mass selective detector. High-resolution mass spectra were obtained on a AccuTOF instrument equipped with a DART ionization source. Melting points were observed in open Pyrex capillary tubes and are uncorrected. Flash chromatography was performed on Silica Gel, 40 micron, 32-63 flash silica. Thin layer chromatography was performed on silica gel. Compounds were visualized by UV, 5% phosphomolybdic acid in ethanol, 0.5% potassium permanganate in water or a solution of ethanol/H₂SO₄/AcOH/p-anisaldehyde (135:5:1.5:3.7).

### Intermediate 1: 3-Hydroxy-2-methylenehexadecanoic acid

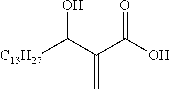

Tetradecanal (4.93 g, 23.2 mmol) and methyl acrylate (4.20 mL, 46.8 mmol) were combined in an empty flask. 3-Hydroxyquinuclidine (0.74 g, 5.81 mmol) was added, followed by MeOH (0.70 mL) The resulting mixture was allowed to stir for 3 d. MeOH and excess methyl acrylate were removed under reduced pressure. The resulting residue was diluted with a H₂O/sat'd aq. NH₄Cl solution (5:1, 120 mL), and the resulting mixture was extracted with CH₂Cl₂ (3×50 mL). The combined organic layers were dried (Na₂SO₄) and concentrated. Methyl 3-hydroxy-2-methylenehexadecanoate was obtained as a white solid (5.82 g, 84%) and used without further purification.

Methyl 3-hydroxy-2-methylenehexadecanoate (5.82 g, 19.5 mmol) was dissolved in EtOH/H₂O (2:1, 45 mL). Lithium hydroxide monohydrate (0.45 g, 19.5 mmol) was added; the resulting solution was allowed to stir overnight. The reaction was quenched with 1 M HCl (200 mL), and the solution was extracted with CH₂Cl₂ (3×100 mL). The combined organic layers were washed with H₂O (100 mL) and dried (MgSO₄); CH₂Cl₂ was removed under reduced pressure. See Martinez et al. Org. Lett. (2003) 5, 399. Purification of the residue by flash chromatography on silica gel (petroleum ether/EtOAc 85:15) gave 3-hydroxy-2-methylenehexadecanoic acid (5.38 g, 95%) as a white solid: mp: 72.4-73.5° C.; IR (KBr) 3853, 2918, 2850, 2594, 2360, 1697, 1637 cm⁻; ¹H NMR (400 MHz, CDCl₃) δ 6.38 (s, 1H), 5.91 (s, 1H), 4.43 (dd, J=6.5, 6.5 Hz, 1H), 1.68-1.66 (m, 2H), 1.43-1.41 (m, 1H), 1.30-1.26 (m, 22H), 0.90 (t, J=6.9 Hz, 3H); ¹³C NMR (100 MHz, CDCl₃) δ 171.4, 142.1, 127.6, 71.8, 36.4, 32.1, 29.9, 29.9, 29.9, 29.8, 29.8, 29.6, 29.6, 26.0, 22.9, 14.3; MS (EI) m/z 266 (M−OH)⁺, 221, 192, 116, 101 (100), 83, 71, 57; HRMS (FAB) calcd for C₁₇H₃₂NaO₃ [M+Na]⁺ m/z 307.2244, found 307.2258.

### Intermediate 2: S-tert-Butyl 3-hydroxy-2-methylenehexadecanethioate

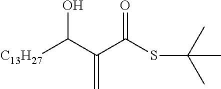

DABCO (0.040 g, 0.37 mmol) was added to a mixture of thio-tert-butyl acrylate. See Zhou et al. Org. Lett. (2007) 9, 4663) (1.14 g, 7.87 mmol) and tetradecanal (0.84 g, 3.7 mmol. The reaction mixture was allowed to stir for a week. It was diluted with CH₂Cl₂ (100 mL) and washed with 1 M aqueous HCl (50 mL), followed by sat. NaHCO₃ (50 mL). The organic layer was dried (Na₂SO₄) and concentrated. Purification by flash chromatography on silica gel (petroleum ether/EtOAc 95:5) gave the desired product (1.03 g, 84%) as slightly yellow oil: IR (neat) 2921, 2852, 1655, 1456, 1363, 967, 721 cm⁻¹; ¹H NMR (400 MHz, CDCl₃) δ 6.05 (s, 1H), 5.71 (d, J=1.0 Hz, 1H), 4.38 (dd, J=6.7, 6.7 Hz, 1H), 2.40 (br. s, 1H), 1.65-1.56 (m, 2H), 1.49 (s, 9H), 1.45-1.41 (m, 1H), 1.33-1.25 (m, 21H), 0.88 (t, J=6.7 Hz, 3H); ¹³C NMR (100 MHz, CDCl₃) δ 195.8, 151.4, 121.7, 72.3, 48.4, 36.4, 32.1, 30.0, 29.9, 29.8, 29.7, 29.6, 29.6, 25.9, 22.9, 14.3; HRMS (ESI) calcd for C₂₁H₄₁O₂S [M+H]⁺ m/z 357.2822, found 357.2822.).

### Intermediate 3: 3-Methylene-4-tridecyloxetan-2-one

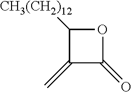

Pathway A (see Martinez et al. Org. Lett. (2003) 5, 399): 3-Hydroxy-2-methylenehexadecanoic acid (5.38 g, 18.9 mmol) was dissolved in dry CH₂Cl₂ (70 mL). Oven-dried Na₂CO₃ (20.1 g, 18.9 mmol) was added, and the resulting suspension was stirred for 30 min. O-Nosyl chloride (8.50 g, 37.7 mmol) was added, and the resulting mixture was stirred for 3 d. The reaction was diluted with CH₂Cl₂ (200 mL), and then 1 M HCl (200 mL) was added and separated. The aqueous layer was then extracted with CH₂Cl₂ (3×75 mL); the combined organic layers were dried (Na₂SO₄) and concentrated. Purification of the residue via flash chromatography on silica gel (petroleum ether/EtOAc 95:5) gave 3-methylene-4-tridecyloxetan-2-one (2.19 g, 43%) as a white solid. Pathway B. See Capozzi et al. J. Org. Chem. (1993) 58, 7932): Hg(CO₂CF₃)₂ (0.72 g, 1.68 mmol), was added to a solution of S-tert-butyl 3-hydroxy-2-methylenehexadecanethioate (Intermediate 2) (0.30 g, 0.84 mmol) in dry CH₃CN (50 mL). The reaction mixture was stirred in a preheated oil bath for 10 min at 50° C. ¹H NMR was used to monitor the reaction. The reaction mixture was filtered, and the filtrate concentrated. Purification of the residue by flash chromatography on silica gel (petroleum ether/EtOAc 95:5) gave 3-methylene-4-tridecyloxetan-2-one (0.15 g, 67%) as a white solid: mp: 34-35.5° C.; IR (neat) 2916, 2849, 1810, 1468, 1085, 962, 818 cm⁻¹; ¹H NMR (400 MHz, CDCl₃) δ 5.90 (dd, J=2.0, 2.0 Hz, 1H), 5.41 (dd, J=1.7 1.7 Hz, 1H), 4.98-4.94 (m, 1H), 1.92-1.79 (m, 2H), 1.54-1.40 (m, 2H), 1.37-1.26 (m, 20H), 0.88 (t, J=6.7 Hz, 3H); ¹³C NMR (100 MHz, CDCl₃) δ 163.9, 146.7, 115.0, 79.9, 33.5, 32.1, 29.9, 29.9, 29.8, 29.7, 29.6, 29.6, 29.5, 24.8, 22.9, 14.3; MS (EI) m/z 266 (M+), 108, 95, 83 (100), 67, 55; HRMS (ESI) calcd for C₁₇H₃₁O₂[M+H]⁺ m/z 267.2319, found 267.2335.

### Intermediate 4: 3-Methylene-4-(9-phenylnonyl)oxetan-2-one

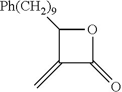

### Step a. Preparation of 1-(tert-Butyldimethylsilyloxy)-10-phenyldec-9-yne

n-Butyllithium (2.5 M in THF, 1.62 mL, 4.05 mmol) was added to a solution of phenyl acetylene (3.43 mL, 31.2 mmol) in THF (10 mL) at 0° C. After 10 min a solution of 1-(tert-butyldimethyl-silyloxy)-8-iodooctane (5.87 g, 15.6 mmol) and HMPA (10 mL) was added drop-wise. The resultant solution was stirred at 0° C. for 30 min, then allowed to slowly warm to rt overnight. Saturated aqueous NH₄Cl (60 mL) was added. The layers were separated, followed by extraction of the aqueous layer with Et₂O (3×30 mL). The combined organic layers were dried (MgSO₄) and concentrated. The crude residue was purified by flash chromatography on silica gel (petroleum ether/EtOAc, 99:1) to yield 1-(tert-butyldimethylsilyloxy)-10-phenyldec-9-yne (A) (2.56 g, 48%) as a colorless oil: IR (neat) 2927, 2855, 1462, 1253, 1093, 834, 774, 754, 691 cm⁻; ¹H NMR (400 MHz, CDCl₃) δ 7.47-7.38 (m, 2H), 7.30-7.23 (m, 3H), 3.61 (t, J=6.6 Hz, 2H), 2.40 (t, J=7.1 Hz, 2H), 1.64-1.57 (m, 2H), 1.53-1.44 (m, 4H) 1.38-1.33 (m, 6H), 0.90 (s, 9H), 0.05 (s, 6H); ¹³C NMR (100 MHz, CDCl₃) δ 131.8, 128.4, 127.6, 124.3, 90.6, 80.8, 63.5, 33.1, 29.6, 29.4, 29.1, 29.0, 26.2, 26.0, 19.6, 18.6, −5.0; HRMS (ESI) calcd for C₂₂H₃₇OSi [M+H]⁺ m/z 345.2608, found 345.2596.

### Step b. Preparation of 1-(tert-Butyldimethylsilyloxy)-10-phenyldecane

Pd/C (10 mol %, 0.27 g, 0.26 mmol) was added to a solution of 1-(tert-butyldimethylsilyloxy)-10-phenyldec-9-yne (2.96 g, 8.59 mmol) in THF (34 mL). The mixture was purged with H₂ for 5 min and then stirred under H₂ for 7.5 h. The reaction mixture was filtered through a pad of celite, and the filtrate was concentrated to provide 1-(tert-butyldimethylsilyloxy)-10-phenyldecane (2.21 g, 74%) as a colorless oil, which was clean based on ¹H and ¹³C NMR and was used without further purification in the next reaction: IR (neat) 2925, 2853, 1462, 1252, 1096, 834, 774, 679 cm⁻¹; ¹H NMR (400 MHz, CDCl₃) δ 7.29-7.25 (m, 2H), 7.18-7.15 (m, 3H), 3.60 (t, J=6.6 Hz, 2H), 2.60 (t, J=7.6 Hz, 2H), 1.65-1.59 (m, 2H), 1.51 (quin, J=6.8 Hz, 2H) 1.34-1.28 (m, 12H), 0.90 (s, 9H), 0.05 (s, 6H); ¹³C NMR (100 MHz, CDCl₃) δ 143.2, 128.6, 128.4, 125.7, 63.6, 36.2, 33.1, 31.7, 29.8, 29.7, 29.7, 29.7, 29.6, 26.2, 26.0, 18.6, −5.0; HRMS (ESI) calcd for C₂₂H₄₀OSi [M+H]⁺ m/z 349.2921, found 349.2919.

### Step c. Preparation of 10-Phenyldecane-1-ol

TBAF (1M in THF, 15.0 mL, 15.0 mmol) was added drop-wise to a stirred solution of 1-(tert-butyldimethylsilyloxy)-10-phenyldecane (2.81 g, 7.5 mmol) in THF (140 mL) at 0° C. The reaction was allowed to warm to rt and stir overnight. Saturated aqueous NH₄Cl (100 mL) was added. The layers were separated, and the aqueous layer was extracted with EtOAc (3×50 mL). The combined organic layers were dried (MgSO₄) and concentrated. The crude residue was purified by flash chromatography on silica gel (petroleum ether/EtOAc, 90:10) to give 10-phenyldecane-1-ol (1.41 g, 81%) as a colorless oil: IR (neat) 3431, 2919, 2848, 1494, 1453, 1348, 1060, 1028, 1006, 741, 717, 694 cm⁻¹; ¹H NMR (400 MHz, CDCl₃) δ 7.31-7.26 (m, 2H), 7.20-7.17 (m, 3H), 3.64 (t, J=6.6 Hz, 2H), 2.60 (t, J=7.6 Hz, 2H), 1.64-1.52 (m, 4H), 1.48 (br. s, 1H), 1.30-1.28 (m, 12H); ¹³C NMR (100 MHz, CDCl₃) δ 143.1, 128.6, 128.4, 125.7, 63.2, 36.2, 32.9, 31.8, 29.8, 29.7, 29.6, 29.5, 25.9; HRMS (ESI) calcd for C₁₆H₂₇O [M+H]⁺ m/z 235.2056, found 235.2065.

### Step d. Preparation of 10-Phenyldecanal

4-Acetamido-2,2,6,6-tetramethylpiperidine-1-oxoammonium tetrafluoroborate (2.01 g, 6.68 mmol) and SiO₂ (2.01 g) were added to a solution of 10-phenyldecane-1-ol (1.41 g, 6.07 mmol) in CH₂Cl₂ (48 mL). The reaction mixture was stirred at rt overnight. The reaction mixture was then filtered through a pad of SiO₂, and the filtrate was concentrated and used without further purification, as both ¹H and ¹³C NMR showed clean 10-phenyldecanal (1.26 g, 90%) as colorless crystals: mp: 41-42° C.; IR (neat) 2931, 2847, 1698, 1411, 1282, 1250, 1218, 942, 744, 696 cm⁻¹; ¹H NMR (400 MHz, CDCl₃) δ 9.76 (t, J=1.8 Hz, 1H), 7.29-7.25 (m, 2H), 7.18-7.15 (m, 3H), 2.60 (t, J=7.5 Hz, 2H), 2.41 (dt, J=7.3, 1.8 Hz, 2H), 1.66-1.57 (m, 4H), 1.30-1.29 (m, 10H); ¹³C NMR (100 MHz, CDCl₃) δ 203.1, 143.1, 128.6, 128.4, 125.8, 44.1, 36.2, 31.7, 29.6, 29.5, 29.5, 29.3, 22.3; HRMS (ESI) calcd for C₁₆H₂₄O [M−H]⁺ m/z 231.1754, found 231.1734.

### Step e. Preparation of Methyl 3-hydroxy-2-methylene-12-phenyldodecanoate

10-Phenyldecanal (1.26 g, 5.45 mmol) and methyl acrylate (0.98 mL, 10.9 mmol) were combined in an empty flask. 3-Hydroxyquinuclidine (0.173 g, 1.36 mmol) was added, followed by MeOH (0.17 mL). The resulting mixture was stirred for 2 d. MeOH and excess methyl acrylate were removed under reduced pressure. The resulting residue was diluted with a H₂O/sat'd aq. NH₄Cl solution (5:1, 30 mL), and the resulting mixture was extracted with CH₂Cl₂ (3×15 mL). The combined organic layers were dried (Na₂SO₄) and concentrated. Purification by flash chromatography on silica gel (petroleum ether/EtOAc, 95:5) gave methyl 3-hydroxy-2-methylene-12-phenyldodecanoate (1.06 g, 61%) as a colorless oil: IR (neat) 3350 (br), 2824, 2853, 1717, 1438, 1266, 1194, 1153, 819, 755 cm⁻¹; ¹H NMR (400 MHz, CDCl₃) δ 7.29-7.25 (m, 2H), 7.19-7.15 (m, 3H), 6.22 (d, J=0.3 Hz, 1H), 5.81 (t, J=0.8 Hz, 1H), 4.39 (dd, J=13.0, 6.7 Hz, 1H), 3.78 (s, 3H), 2.62-2.56 (m, 3H), 1.69-1.57 (m, 4H), 1.48-1.43 (m, 1H), 1.30-1.28 (m, 11H); ¹³C NMR (100 MHz, CDCl₃) δ 167.2, 143.1, 142.7, 128.6, 128.4, 125.7, 125.1, 72.0, 52.0, 36.4, 36.2, 31.7, 29.7, 29.7, 29.7, 29.6, 29.5, 26.0; HRMS (ESI) calcd for C₂₀H₃₁O₃[M+H]⁺ m/z 319.2268, found 319.2281.

### Step f. Preparation of 3-Hydroxy-2-methylene-12-phenyldodecanoic acid

Methyl 3-hydroxy-2-methyl-ene-12-phenyldodecanoate (1.06 g, 3.37 mmol) was dissolved in EtOH/H₂O (2:1, 8 mL). Lithium hydroxide (77 mg, 3.4 mmol) was added; the resulting solution was stirred overnight. See Knapp et al. J. Org. Chem. (1988) 53, 4006. The reaction was quenched with 1 M HCl (30 mL), and the solution was extracted with CH₂Cl₂ (3×20 mL). The combined organic layers were washed with H₂O (30 mL) and dried (MgSO₄); CH₂Cl₂ was removed under reduced pressure. Purification of the residue by flash chromatography on silica gel (petroleum ether/EtOAc, 85:15) gave 3-hydroxy-2-methylene-12-phenyldodecanoic acid (0.396 g, 54%) as a colorless oil: IR (neat) 3853, 2919, 2848, 1685, 1622, 1493, 1435, 1284, 1181, 1118, 1070, 1025, 967, 747, 695 cm⁻; ¹H NMR (400 MHz, CDCl₃) δ 7.29-7.25 (m, 2H), 7.18-7.15 (m, 3H), 6.38 (s, 1H), 5.91 (s, 1H), 4.42 (dd, J=6.3, 6.3 Hz, 1H), 2.60 (t, J=7.6 Hz, 2H), 1.74-1.57 (m, 4H), 1.48-1.43 (m, 1H), 1.30-1.28 (m, 11H); ¹³C NMR (100 MHz, CDCl₃) δ 170.8, 143.2, 142.0, 128.6, 128.4, 127.6, 125.8, 71.8, 36.4, 36.2, 31.7, 29.7, 29.7, 29.6, 29.5, 26.0; HRMS (ESI) calcd for C₁₉H₂₈O₃[M+H]⁺ m/z 304.2038, found 305.2111.

### Step g. Preparation of 3-Methylene-4-(9-phenylnonyl)oxetan-2-one

3-Hydroxy-2-methylene-12-phenyl-dodecanoic acid (0.40 g, 1.8 mmol) was dissolved in dry CH₂Cl₂ (7 mL). Oven-dried Cs₂CO₃ (0.64 g, 1.8 mmol) was added, and the resulting suspension was stirred for 20 min. o-Nosyl chloride (800 mg, 3.6 mmol) was added, and the resulting mixture was stirred for 3 d. The reaction was diluted with CH₂Cl₂ (20 mL), and then 1 M HCl (20 mL) was added. The aqueous layer was separated and extracted with CH₂Cl₂ (3×15 mL). The combined organic layers were dried (Na₂SO₄) and concentrated. Purification of the residue by flash chromatography on silica gel (petroleum ether/EtOAc, 95:5) gave 3-methylene-4-(9-phenylnonyl)oxetan-2-one (0.089 g, 17%) as a colorless oil: IR (neat) 2926, 2854, 1821, 1080 cm⁻¹; ¹H NMR (400 MHz, CDCl₃) δ 7.27-7.23 (m, 2H), 7.17-7.13 (m, 3H), 5.87 (dd, J=1.8, 1.8 Hz, 1H), 5.38 (dd, J=1.5, 1.5 Hz, 1H), 4.95-4.91 (m, 1H), 2.59 (t, J=7.6 Hz, 2H), 1.88-1.78 (m, 2H), 1.60 (quin, J=8.0 Hz, 2H), 1.50-1.40 (m, 2H), 1.38-1.29 (m, 10H); ¹³C NMR (100 MHz, CDCl₃) δ 163.8, 146.6, 143.0, 128.5, 128.4, 125.7, 115.0, 79.8, 36.1, 33.4, 31.6, 29.6, 29.5, 29.5, 29.4, 29.4, 24.7; HRMS (ESI) calcd for C₁₉H₂₇O₂[M+H]⁺ m/z 287.2006, found 287.1999.

### Intermediate 5: Hept-6-enamide

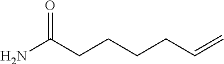

A solution of 6-heptenoic acid (0.50 g, 3.9 mmol), SOCl₂ (0.34 mL, 4.7 mmol), and a drop of DMF in CHCl₃ (6 mL) was refluxed for 2 h. The reaction mixture was cooled to rt and then poured into a mixture of aqueous NH₄OH (28-30%, 6 mL) and ice (6.3 g) and stirred for 2 h. The layers were separated, and the organic layer was dried (Na₂SO₄) and concentrated. Purification by flash column chromatography on silica gel (CH₂Cl₂/MeOH, 98:2) gave hept-6-enamide (0.42 g, 85%) as a white solid: mp: 84.8-84.9° C.; IR (neat) 3355, 3178, 2938, 1630, 1460, 1410, 1221 cm⁻¹; ¹H NMR (400 MHz, CDCl₃) δ 6.32 (br s, 1H), 5.84 (br s, 1H), 5.79-5.69 (m, 1H), 4.95 (d, J=17.2 Hz, 1H), 4.88 (d, J=10.2 Hz, 1H), 2.17 (t, J=7.2 Hz, 2H), 2.02 (q, J=6.4 Hz, 2H), 1.60 (quin, J=7.5 Hz, 2H), 1.39 (quin, J=7.6 Hz, 2H); ¹³C NMR (100 MHz, CDCl₃) δ 176.3, 138.5, 114.8, 35.9, 33.5, 28.5, 25.1 MS (EI) m/z 127 (M+), 72, 59 (100); HRMS (ESI) calcd for C₇H₁₄NO [M+H]⁺ m/z 128.1070, found 128.1057.

### Intermediate 6: Hept-6-ene-1-sulfonamide

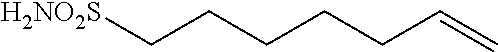

A solution of 7-bromo-1-heptene (0.50 g, 4.3 mmol), and sodium sulfite (0.65 g, 5.1 mmol) in H₂O (3.0 mL) was refluxed overnight. See Dauban et al. Org. Lett. (2000) 2, 2327. After cooling to rt the aqueous solution was washed with Et₂O (2.2 mL) before being evaporated to dryness. The resulting white solid was dried under vacuum at 130° C. for 1 h and then POCl₃ (4.3 mL) was added, and the mixture was stirred for 4 h at 130° C. The reaction mixture was evaporated, and the residue was taken up in CH₃CN (5 mL), and a solution of NH₄OH (28.0-30.0%, 10 mL) in CH₃CN (4 mL) was slowly added at 0° C. The reaction mixture was stirred at 0° C. for 1 h before being diluted with CH₂Cl₂ (30 mL) and washed with H₂O (20 mL). The organic layer was dried (Na₂SO₄) and concentrated. Purification by flash column chromatography on silica gel (petroleum ether/EtOAc, 80:20) gave hept-6-ene-1-sulfonamide (0.29 g, 42%) as a white solid: mp: 52-52.5° C.; IR (neat) 3338, 3253, 2927, 1469, 1294, 1134, 993, 922, 885, 807 cm⁻¹; ¹H NMR (400 MHz, CDCl₃) δ 5.78 (ddt, J=17.0, 10.2, 6.4 Hz, 1H), 5.03-4.94 (m, 2H), 4.84 (br s, 2H), 3.13-3.09 (m, 2H), 2.06 (q, J=6.3 Hz, 2H), 1.89-1.81 (m, 2H), 1.45-1.43 (m, 4H); ¹³C NMR (100 MHz, CDCl₃) δ 138.5, 114.9, 55.3, 33.4, 28.4, 27.7, 23.8; HRMS (ESI) calcd for C₇H₁₆NO₂S [M+H]⁺ m/z 178.0896, found 178.0913.

### Example 1: (E/Z)-3-Octadecylidene-4-tridecyloxetan-2-one

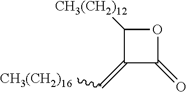

Catalyst A (40 mg, 0.047 mmol) was added to a solution of 3-methylene-4-tridecyloxetan-2-one (Intermediate 3) (0.25 g, 0.94 mmol) and 1-nonadecene (0.38 g, 1.4 mmol) in dry CH₂Cl₂ (46 mL). The mixture was stirred overnight at 40° C. See Raju et al. Org. Lett. (2006) 8, 2139. The next day ¹H NMR showed complete consumption of Intermediate 3. The reaction was allowed to cool to rt followed by removal of CH₂Cl₂ under reduced pressure to yield a brownish residue. Purification of the residue by flash chromatography on silica gel (petroleum ether/EtOAc 98:2) gave (Z/E)-3-octadecylidene-4-tridecyloxetan-2-one, (Z/E, 2.2/1), (0.56 g, 75%) as a white solid. The isomers were separated by careful chromatography using the same solvent system. E-isomer (1a): mp 49-50° C.; IR (neat) 2917, 2849, 1791, 1466, 1127 cm⁻¹; ¹H NMR (400 MHz, CDCl₃) δ 6.33 (dt, J=7.9, 1.4 Hz, 1H), 4.99 (m, 1H), 2.10 (dt, J=7.4, 7.4 Hz, 2H), 2.00-1.88 (m, 1H), 1.82-1.72 (m, 1H), 1.52-1.43 (m, 4H), 1.37-1.26 (m, 48H), 0.88 (t, J=6.5 Hz, 6H); ¹³C NMR (100 MHz, CDCl₃) δ 164.6, 137.8, 134.2, 79.4, 33.5, 32.1, 29.9, 29.9, 29.9, 29.9, 29.8, 29.7, 29.7, 29.7, 29.6, 29.5, 29.5, 29.4, 29.1, 28.6, 24.9, 22.9, 14.3; MS (EI) m/z 194,117, 91 (100), 77, 65, 51; HRMS (ESI) calcd for C₃₄H₆₅O₂[M+H]⁺ m/z 505.4957, found 505.5003. Z-isomer (1b): mp 61.5-62.5° C.; IR (neat) 2914, 2848, 1795, 1471, 1117, 1070 cm⁻¹; ¹H NMR (400 MHz, CDCl₃) δ 5.84 (t, J=7.5 Hz, 1H), 4.85 (t, J=6.3 Hz, 1H), 2.48 (dt, J=7.4, 7.4 Hz, 2H), 1.86-1.73 (m, 2H), 1.47-1.39 (m, 4H), 1.35-1.26 (m, 48H), 0.88 (t, J=6.5 Hz, 6H); ¹³C NMR (100 MHz, CDCl₃) δ 164.4, 137.7, 136.5, 78.9, 34.0, 32.1, 29.9, 29.9, 29.8, 29.8, 29.7, 29.7, 29.6, 29.6, 29.5, 29.5, 29.5, 29.3, 29.1, 29.1, 24.8, 22.9, 14.3; MS (EI) m/z 460 (M-CO₂)⁺, 355, 341, 293, 281, 236, 207 (100), 194, 110, 95, 81, 67, 55; HRMS (ESI) calcd for C₃₄H₆₅O₂ [M+H]⁺ m/z 505.4957, found 505.4957.

Catalyst A is

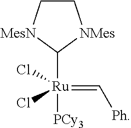

### Example 2: (E/Z)-3-Hexylidene-4-tridecyloxetan-2-one

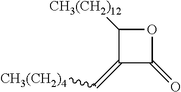

Catalyst A (0.02 g, 0.04 mmol) was added to a solution of 3-methylene-4-tridecyloxetan-2-one (Intermediate 3) (0.20 g, 0.75 mmol) and 1-heptene (0.15 g, 1.5 mmol) in dry CH₂Cl₂ (29 mL). The mixture was stirred overnight at 40° C. The next day ¹H NMR showed complete consumption of 3-methylene-4-tridecyloxetan-2-one. The reaction was cooled to rt followed removal of the CH₂Cl₂ under reduced pressure to yield a brownish residue. Purification of the residue by flash chromatography on silica gel (petroleum ether/EtOAc 98:2) gave (Z/E)-3-hexylidene-4-tridecyloxetan-2-one (Z/E, 3.3/1), (0.24 g, 97%) as a colorless oil. The isomers were separated by careful chromatography using the same solvent system. E-isomer (2a): IR (neat) 2922, 2852, 1813, 1464, 1117 cm⁻¹; ¹H NMR (400 MHz, CDCl₃) δ 6.33 (t, J=7.6 Hz, 1H), 4.99 (m, 1H), 2.11 (dt, J=7.1, 7.1 Hz, 2H), 1.96-1.88 (m, 1H), 1.81-1.72 (m, 1H), 1.50-1.43 (m, 4H), 1.31-1.26 (m, 24), 0.90 (m, 3H), 0.88 (t, J=7.5 Hz, 3H); ¹³C NMR (100 MHz, CDCl₃) δ 164.6, 137.8, 134.1, 79.4, 33.5, 32.1, 31.6, 29.9, 29.9, 29.8, 29.7, 29.6, 29.6 29.5, 29.0, 28.3, 24.9, 22.9, 22.6, 14.3, 14.1; MS (EI) m/z 336 (M⁺), 178 (100); HRMS (ESI) calcd for C₂₂H₄₁O₂ [M+H]⁺ m/z 337.3101, found 337.3124. Z-isomer (2b): IR (neat) 2914, 2848, 1793, 1721, 1470, 1116, 1070, cm⁻¹; ¹H NMR (400 MHz, CDCl₃) δ 5.84 (dt, J=7.9, 1.0 Hz, 1H), 4.84 (dd, J=6.2, 6.2 Hz, 1H), 2.55-2.41 (m, 2H), 1.84-1.73 (m, 2H), 1.50-1.39 (m, 4H), 1.33-1.26 (m, 24H), 0.89 (m, 3H), 0.88 (t, J=7.0 Hz 3H); ¹³C NMR (100 MHz, CDCl₃) δ 164.4, 137.7, 136.4, 78.8, 33.9, 32.1, 31.4, 29.9, 29.9, 29.8, 29.7, 29.6, 29.6, 29.5, 29.0, 28.7, 24.8, 22.9, 22.6, 14.3, 14.1; MS (EI) m/z 336 (M+), 275, 111, 81, 67, 55 (100); HRMS (ESI) calcd for C₂₂H₄₁O₂[M+H]⁺ m/z 337.3101, found 337.3123.

### Example 3: (Z)-3-Benzylidene-4-tridecyloxetan-2-one

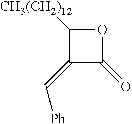

Catalyst A (0.01 g, 0.01 mmol) was added to a solution of 3-methylene-4-tridecyloxetan-2-one (Intermediate 3) (0.060 g, 0.21 mmol) and styrene (0.050 g, 0.43 mmol) in dry CH₂Cl₂ (8 mL) The mixture was stirred overnight at 40° C. The next day TLC showed incomplete consumption of Intermediate 3; so 1 equiv more of styrene was added, and the reaction was allowed to stir for 6 h at 40° C. After the reaction mixture was allowed to cool to rt, the CH₂Cl₂ was removed under reduced pressure to yield a brown residue. Purification of the residue by flash chromatography on silica gel (petroleum ether/EtOAc 98:2) gave (Z)-3-benzylidene-4-tridecyloxetan-2-one (0.030 g, 40%) as a white solid: mp 61-62° C.; IR (neat) 2914, 2848, 1780, 1687, 1468, 1212, 1156, 1128, 1068 cm⁻; ¹H NMR (400 MHz, CDCl₃) δ 7.95 (d, J=6.8 Hz, 2H), 7.43-7.41 (m, 3H), 6.53 (s, 1H), 4.96 (dd, J=5.6, 5.6 Hz, 1H), 1.97-1.83 (m, 2H), 1.59-1.45 (m, 2H), 1.40-1.26 (m, 20H), 0.88 (t, J=6.6 Hz, 3H); ¹³C NMR (100 MHz, CDCl₃) δ 163.3, 136.0, 133.4, 133.2, 130.8, 130.4, 129.1, 78.0, 34.0, 32.1, 29.9, 29.9, 29.8, 29.7, 29.6, 29.6, 24.8, 22.9, 14.3; MS (EI) m/z 342 (M⁺), 159, 130 (100); HRMS (ESI) calcd for C₂₃H₃₅O₂[M+H]⁺ m/z 343.2632, found 343.2655.

### Example 4: (Z)-3-(5-Phenylpentylidene)-4-tridecyloxetan-2-one

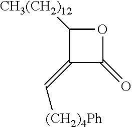

Catalyst A (0.03 g, 0.04 mmol) was added to a solution of 3-methylene-4-tridecyloxetan-2-one (Intermediate 3) (0.20 g, 0.75 mmol) and 6-phenyl-1-hexene (0.24 g, 1.50 mmol) in dry CH₂Cl₂ (29 mL). The mixture was stirred overnight at 40° C. The next day TLC showed incomplete consumption of Intermediate 3; so 1 equiv more of 6-phenyl-1-hexene was added, and the reaction was allowed to stir for 6 h at 40° C. After the reaction was allowed to cool to rt, the CH₂Cl₂ was removed under reduced pressure to yield a brown residue. Purification of the residue by flash chromatography on silica gel (petroleum ether/EtOAc 97:3) gave (Z)-3-(5-phenylpentylidene)-4-tridecyloxetan-2-one (0.20 g, 68%) as a wax: mp 34-35° C.; IR (neat) 2914, 2848, 1793, 1723, 1468, 1182.70, 1120, 1071 cm⁻¹; ¹H NMR (400 MHz, CDCl₃) δ 7.22-7.19 (m, 2H), 7.13-7.09 (m, 3H), 5.75 (t, J=7.8 Hz, 1H), 4.77 (dd, J=6.0, 6.0 Hz, 1H), 2.56 (t, J=7.2 Hz, 2H), 2.51-2.38 (m, 2H), 1.77-1.69 (m, 2H), 1.60 (quin, J=7.6 Hz, 2H), 1.47-1.33 (m, 4H), 1.28-1.19 (m, 20H), 0.81 (t, J=6.8 Hz, 3H); ¹³C NMR (100 MHz, CDCl₃) δ 164.4, 142.4, 138.0, 136.0, 128.6, 128.5, 126.0, 78.9, 35.7, 33.9, 32.1, 30.9, 29.9, 29.9, 29.8, 29.7, 29.6, 29.6, 29.5, 28.9, 28.5, 24.8, 22.9, 14.3; MS (EI) m/z 354 (M-CO₂)+, 104, 91(100); HRMS (ESI) calcd for C₂₇H₄₃O₂[M+H]⁺ m/z 399.3263, found 399.3258.

### Example 5: (Z)-3-(4-Phenylbutylidene)-4-tridecyloxetan-2-one

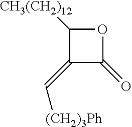

Catalyst A (0.02 g, 0.04 mmol) was added to a solution of 3-methylene-4-tridecyloxetan-2-one (Intermediate 3) (0.20 g, 0.75 mmol) and 5-phenyl-1-pentene (0.44 g, 3.00 mmol) in dry CH₂Cl₂ (29 mL). The mixture was stirred overnight at 40° C. The next day TLC showed incomplete consumption of Intermediate 3; so 1 equiv more of 5-phenyl-1-pentene was added and the reaction was allowed to stir for 6 h at 40° C. After the reaction was allowed to cool to rt, the CH₂Cl₂ was removed under reduced pressure to yield a brown residue. Purification of the residue by flash chromatography on silica gel (petroleum ether/EtOAc 97:3) gave (Z)-3-(4-phenylbutylidene)-4-tridecyloxetan-2-one (0.15 g, 51%) as a colorless oil: IR (neat) 2921, 2852, 1805, 1454, 1067 cm⁻¹; ¹H NMR (400 MHz, CDCl₃) δ 7.31-7.27 (m, 2H), 7.21-7.18 (m, 3H), 5.84 (t, J=7.6 Hz, 1H), 4.86 (dd, J=6.0, 6.0 Hz, 1H), 2.67 (t, J=7.2 Hz, 2H), 2.56 (dt, J=7.8, 7.8 Hz, 2H), 1.84-1.77 (m, 4H), 1.50-1.43 (m, 2H), 1.36-1.27 (m, 20H), 0.89 (t, J=6.5 Hz, 3H); ¹³C NMR (100 MHz, CDCl₃) δ 164.3, 141.8, 138.1, 135.8, 128.6, 126.2, 78.9, 35.6, 33.9, 32.1, 30.8, 29.9, 29.9, 29.8, 29.7, 29.6, 29.6, 29.5, 28.8, 24.8, 22.9, 14.3; MS (EI) m/z 384 (M+), 104 (100), 91; HRMS (ESI) calcd for C₂₆H₄₁O₂[M+H]⁺ m/z 385.3101, found 385.3114.

### Example 6: (cis/trans)-3-Octadecyl-4-tridecyloxetan-2-one

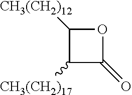

(E/Z)-3-Octadecylidene-4-tridecyloxetan-2-one (0.10 g, 0.20 mmol) (Example 1) was dissolved in a mixture of THF:MeOH (1.6:0.32 mL). This solution was cooled to −10° C., followed by the addition of CoCl₂(PPh)₃ (0.02 g, 0.04 mmol) and then portion-wise addition of NaBH₄ (39.0 mg, 1.2 mmol) within 10 min. See Moritani et al. Bull. Chem. Soc. Jpn (1996) 69, 2286. The mixture was vigorously stirred for 2 h between −7 and −5° C. The reaction mixture was filtered through a pad of celite, and the celite was then washed with CHCl₃ (10 mL). The filtrate was washed with 2M HCl (10 mL), dried (MgSO₄) and concentrated. Purification by flash chromatography on silica gel (petroleum ether/EtOAc, 98:2) gave a mixture of (cis/trans)-3-octadecyl-4-tridecyloxetan-2-one (trans/cis 2/1), (0.06 g, 61%) as a white solid. The isomers were separated by careful chromatography using the same solvent system. cis-isomer (6a): mp 63-64° C.; IR (neat) 2916, 2849, 1790, 1464, 1146, 1076 cm⁻¹; ¹H NMR (400 MHz, CDCl₃) δ 4.52 (ddd, J=9.9, 6.3, 4.1 Hz, 1H), 3.59 (ddd, J=8.1, 6.9, 6.9 Hz, 1H), 1.82-1.72 (m, 2H), 1.69-1.55 (m, 2H), 1.52-1.48 (m, 2H), 1.39-1.26 (m, 52H), 0.88 (t, J=7.0 Hz, 6H); ¹³C NMR (100 MHz, CDCl₃) δ 172.6, 76.0, 52.9, 32.1, 30.4, 29.9, 29.9, 29.9, 29.8, 29.7, 29.6, 29.6, 29.6, 29.5, 27.8, 25.8, 24.2, 22.9, 14.3; MS (EI) m/z 463 (M-CO₂)+, 281, 207, 111, 97(100), 83, 57; HRMS (ESI) calcd for C₃₄H₆₇O₂[M+H]⁺ m/z 507.5136, found 507.5158. trans-isomer (6b) (nocardiolactone): mp 64-65° C. (Lit. 66-68° C.). See Mikami, et al. Nat. Prod. Lett. (1999) 13, 277); IR (neat) 2915, 2847, 1796, 1468, 1154, cm⁻¹; ¹H NMR (400 MHz, CDCl₃) δ 4.21 (ddd, J=6.8, 6.8, 3.9 Hz, 1H), 3.17 (ddd, J=10.3, 6.5, 4.0 Hz, 1H), 1.89-1.79 (m, 2H), 1.76-1.66 (m, 2H), 1.45-1.25 (m, 54H), 0.88 (t, J=7.0 Hz, 6H); ¹³C NMR (100 MHz, CDCl₃) δ 171.9, 78.4, 56.4, 34.7, 32.1, 29.9, 29.9, 29.8, 29.7, 29.7, 29.6, 29.6, 29.5, 29.5, 28.1, 27.2, 25.2, 22.9, 14.3; MS (EI) m/z 462 (M-CO₂)⁺, 281, 207, 111, 97 (100), 83, 57; HRMS (ESI) calcd for C₃₄H₆₇O₂[M+H]⁺ m/z 507.5136, found 507.5157.

### Example 7: (cis/trans)-3-3-Hexyl-4-tridecyloxetan-2-one

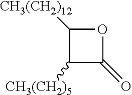

(E/Z)-3-Hexylidene-4-tridecyloxetan-2-one (Example 2) (0.03 g, 0.09 mmol) was dissolved in a mixture of THF:MeOH (0.70 mL: 0.14 mL). This solution was cooled to −10° C., followed by the addition of CoCl₂(PPh)₃ (0.01 g, 0.01 mmol) and then portion-wise addition of NaBH₄ (9.9 mg, 27.0 mmol) within 10 min. The mixture was vigorously stirred for 2 h between −7 to −5° C. The reaction mixture was filtered through a pad of celite, and the celite was then washed with CHCl₃ (5 mL). The filtrate was washed with 2M HCl (5 mL), dried (MgSO₄) and concentrated. Purification by flash chromatography on silica gel (petroleum ether/EtOAc 98:2) gave mixture of (cis/trans)-3-3-hexyl-4-tridecyloxetan-2-one (trans/cis, 1.3/1), (14 mg, 44%). The isomers were separated by careful column chromatography using the same solvent system. cis-isomer (7a) (colorless oil): IR (neat) 2921, 2852, 1820, 1464, 1121 cm⁻¹; ¹H NMR (400 MHz, CDCl₃) δ 4.57-4.49 (m, 1H), 3.61-3.56 (m, 1H), 1.78-1.51 (m, 6H), 1.39-1.26 (m, 28H), 0.91-0.87 (m, 6H); ¹³C NMR (100 MHz, CDCl₃) δ 172.6, 76.0, 52.9, 32.1, 31.7, 30.4, 29.9, 29.9, 29.7, 29.6, 29.6, 29.5, 29.3, 27.8, 25.8, 24.2, 22.9, 22.8, 14.3, 14.2; MS (EI) m/z 294 (M-CO₂)+, 207, 125, 111, 97 (100), 83, 69, 55; HRMS (ESI) calcd for C₂₂H₄₃O₂[M+H]⁺ m/z 339.3258, found 339.3295. trans-isomer (7b) (waxy solid): mp 29-30° C.; IR (neat) 2918, 2850, 1794, 1466, 1142, 1076 cm⁻¹; ¹H NMR (400 MHz, CDCl₃) δ 4.24-4.17 (m, 1H), 3.18-3.13 (m, 1H), 1.85-1.79 (m, 2H), 1.73-1.69 (m, 2H), 1.43-1.26 (m, 30H), 0.89-0.86 (m, 6H); ¹³C NMR (100 MHz, CDCl₃) δ 171.9, 78.4, 56.4, 34.7, 32.1, 31.7, 29.9, 29.9, 29.8, 29.7, 29.6, 29.6, 29.5, 29.2, 28.1, 27.2, 25.2, 22.9, 22.7, 14.3, 14.2; MS (EI) m/z 294 (M-CO₂)⁺, 281, 207, 125, 11, 97, 83 (100), 69, 55; HRMS (ESI) calcd for C₂₂H₄₃O₂ [M+H]⁺ m/z 339.3258, found 339.3296.

### Example 8: (cis/trans)-3-Benzyl-4-tridecyloxetan-2-one

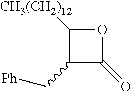

(Z)-3-Benzylidene-4-tridecyloxetan-2-one (Example 3) (0.05 g, 0.15 mmol) was dissolved in a mixture of THF:MeOH (1.1 mL:0.05 mL). This solution was cooled to −10° C., followed by the addition of CoCl₂(PPh)₃ (0.02 g, 0.03 mmol) and then portion-wise addition of NaBH₄ (16.0 mg, 0.44 mmol) within 10 min. The mixture was vigorously stirred for 2 h between −7 to −5° C. The reaction mixture was filtered through a pad of celite, and the celite was then washed with CHCl₃ (5 mL). The filtrate was washed with 2M HCl (5 mL), dried (MgSO₄) and concentrated. Purification by flash chromatography on silica gel (petroleum ether/EtOAc 99:1) gave (cis/trans)-3-benzyl-4-tridecyloxetan-2-one (trans/cis, 2/1), (0.02 g, 39%) as a wax. The isomers were separated by careful column chromatography using the same solvent system. cis-isomer (8a) (white solid): mp 43-44° C.; IR (neat) 2917, 2849, 1797, 1466, 1136, 1067 cm⁻¹; ¹H NMR (400 MHz, CDCl₃) δ 7.34-7.30 (m, 2H), 7.24-7.21 (m, 3H), 4.60 (ddd, J=10.0, 6.4, 3.6 Hz, 1H), 4.01 (ddd, J=9.0, 6.9, 6.9 Hz, 1H), 3.19 (dd, J=15.1, 7.1 Hz, 1H), 2.98 (dd, J=15.1, 9.0 Hz, 1H), 1.86-1.76 (m, 1H), 1.71-1.63 (m, 1H), 1.57-1.46 (m, 1H), 1.37-1.26 (m, 21H), 0.88 (t, J=6.6 Hz, 3H); ¹³C NMR (100 MHz, CDCl₃) δ 171.7, 137.9, 129.0, 128.6, 127.0, 76.3, 53.5, 32.1, 30.6, 29.9, 29.9, 29.8, 29.7, 29.6, 29.6, 29.5, 25.8, 22.9, 14.3; MS (EI) m/z 300 (M-CO₂)⁺, 117, 104 (100), 91; HRMS (ESI) calcd for C₂₃H₃₇O₂[M+H]⁺ m/z 345.2788, found 345.2817. trans-isomer (8b) (white solid): mp 40-41° C.; IR (neat) 2916, 2849, 1800, 1134, 1072 cm⁻¹; ¹H NMR (400 MHz, CDCl₃) δ 7.34-7.30 (m, 2H), 7.27-7.25 (m, 2H), 7.20 (d, J=7.0 Hz, 1H), 4.27 (ddd, J=6.7, 6.7, 4.1 Hz, 1H), 3.45 (ddd, J=9.5, 5.6, 4.2 Hz, 1H), 3.18 (dd, J=14.3, 5.7 Hz, 1H), 3.00 (dd, J=14.3, 9.4 Hz, 1H), 1.83-1.74 (m, 1H), 1.62-1.57 (m, 1H), 1.35-1.18 (m, 22H), 0.88 (t, J=7.0 Hz, 3H); ¹³C NMR (100 MHz, CDCl₃) δ 171.0, 137.4, 129.1, 128.9, 127.3, 77.8, 57.6, 34.4, 34.0, 32.1, 29.9, 29.9, 29.8, 29.6, 29.6, 29.6, 29.3, 24.8, 22.9, 14.3; MS (EI) m/z 300 (M-CO₂)⁺, 117, 104 (100), 91; HRMS (ESI) calcd for C₂₃H₃₇O₂[M+H]⁺ m/z 345.2788, found 345.2814.

### Example 9: (cis/trans)-3-(5-Phenylpentyl)-4-tridecyloxetan-2-one

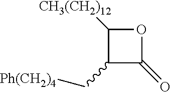

(Z)-3-(5-Phenylpentylidene)-4-tridecyloxetan-2-one (Example 4) (0.10 g, 0.25 mmol) was dissolved in a mixture of THF:MeOH (2.0:0.4 mL). This solution was cooled to −10° C., followed by the addition of CoCl₂(PPh)₃ (0.03 g, 0.04 mmol) and then portion-wise addition of NaBH₄ (28.0 mg, 0.75 mmol), within 10 min. The mixture was vigorously stirred for 4 h between −7 to −5° C. The reaction mixture was filtered through a pad of celite, and the celite was washed with CHCl₃ (15 mL). The filtrate was washed with 2M HCl (10 mL), dried (MgSO₄) and concentrated. Purification by flash chromatography on silica gel (petroleum ether/EtOAc 99:1) gave a mixture of (cis/trans)-3-(5-phenylpentyl)-4-tridecyloxetan-2-one (trans/cis, 2/1), (0.03 g, 32%) as a colorless oil. The isomers were separated by careful column chromatography using the same solvent system. cis-isomer (9a): IR (neat) 2916, 2846, 1805, 1464, 1135, 1064 cm⁻¹; ¹H NMR (400 MHz, CDCl₃) δ 7.29-7.26 (m, 2H), 7.20-7.16 (m, 3H), 4.55-4.49 (m, 1H), 3.57 (ddd, J=8.9, 6.8, 6.8 Hz, 1H), 2.61 (t, J=7.4 Hz, 2H), 1.82-1.70 (m, 2H), 1.68-1.52 (m, 4H), 1.45-1.26 (m, 26H), 0.88 (t, J=6.5 Hz, 3H); ¹³C NMR (100 MHz, CDCl₃) δ 172.5, 142.7, 128.6, 128.5, 125.9, 75.9, 52.8, 36.0, 32.1, 31.3, 30.4, 29.9, 29.9, 29.8, 29.7, 29.6, 29.6, 29.5, 29.2, 27.7, 25.8, 24.1, 22.9, 14.3; MS (GC) m/z 356 (M-CO₂)+, 117, 104 (100), 91; HRMS (ESI) calcd for C₂₇H₄₅O₂[M+H]⁺ m/z 401.3414, found 401.3441. trans-isomer (9b): IR (neat) 2921, 2852, 1819, 1454, 1114 cm⁻¹; ¹H NMR (400 MHz, CDCl₃) δ 7.32-7.28 (m, 2H), 7.20 (m, 3H), 4.23-4.19 (m, 1H), 3.19-3.14 (m, 1H), 2.64 (t, J=7.1 Hz, 2H), 1.87-1.80 (m, 2H), 1.74-1.63 (m, 4H), 1.50-1.29 (m, 26H), 0.91 (br. t, J=6.4 Hz, 3H); ¹³C NMR (100 MHz, CDCl₃) δ 171.8, 142.6, 128.6, 128.5, 125.9, 78.3, 56.3, 36.0, 34.6, 32.1, 31.3, 29.9, 29.9, 29.8, 29.7, 29.6, 29.6, 29.5, 29.1, 28.0, 27.1, 25.2, 22.9, 14.3; MS (EI) m/z 356 (M-CO₂)+, 117, 104 (100), 91; HRMS (ESI) calcd for C₂₇H₄₅O₂ [M+H]⁺ m/z 401.3414, found 401.3447.

### Example 10: (cis/trans)-3-(4-Phenylbutyl)-4-tridecyloxetan-2-one

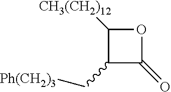

(Z)-3-(4-Phenylbutylidene)-4-tridecyloxetan-2-one (Example 5) (0.05 g, 0.13 mmol)) was dissolved in a mixture of THF:MeOH (1:0.2 mL). This solution was cooled to −10° C., followed by the addition of CoCl₂(PPh)₃ (15.0 mg, 0.02 mmol) and then portion-wise addition of NaBH₄ (14 mg, 0.39 mmol) within 10 min. The mixture was vigorously stirred for 1.5 h between −7 to −5° C. The reaction mixture was filtered through a pad of celite, and the celite was washed with CHCl₃ (10 mL). The filtrate was washed with 2M HCl (5 mL), dried (MgSO₄) and concentrated. Purification by flash chromatography on silica gel (petroleum ether/EtOAc 98:2) gave (cis/trans)-3-(4-phenylbutyl)-4-tridecyloxetan-2-one (trans/cis, 1.4/1), (24 mg, 46%) as a colorless oil. The isomers were separated by careful column chromatography using the same solvent system. cis-isomer (10a): IR (neat) 2919, 2850, 1818, 1462, 1052 cm⁻¹; ¹H NMR (400 MHz, CDCl₃) δ 7.29-7.26 (m, 2H), 7.20-7.16 (m, 3H), 4.51 (ddd, J=10.0, 6.2, 4.0 Hz, 1H), 3.58 (ddd, J=8.6, 6.7, 7.7 Hz, 1H), 2.63 (t, J=7.5 Hz, 2H), 1.85-1.77 (m, 1H), 1.75-1.62 (m, 6H), 1.51-1.26 (m, 21H), 0.88 (t, J=6.5 Hz, 3H); ¹³C NMR (100 MHz, CDCl₃) δ 172.4, 142.3, 128.6, 126.0, 75.9, 52.8, 35.8, 32.1, 31.3, 30.4, 29.9, 29.9, 29.8, 29.7, 29.6, 29.6, 29.5, 27.4, 25.7, 24.0, 22.9, 14.3; MS (EI) m/z 342 (M−CO₂), 117, 104 (100), 91; HRMS (ESI) calcd for C₂₆H₄₃O₂[M+H]⁺ m/z 387.3258, found 387.3261. trans-isomer (10b): IR (neat) 2917, 2851, 1804, 1470, 1140 cm⁻¹; ¹H NMR (400 MHz, CDCl₃) δ 7.30-7.26 (m, 2H), 7.20-7.15 (m, 3H), 4.19 (ddd, J=6.5, 6.5, 3.9 Hz, 1H), 3.15 (m, 1H), 2.63 (t, J=7.5 Hz, 2H), 1.90-1.81 (m, 2H), 1.79-1.62 (m, 4H), 1.51-1.26 (m, 24H), 0.88 (t, J=7.0 Hz, 3H); ¹³C NMR (100 MHz, CDCl₃) δ 171.8, 142.3, 128.6, 126.1, 78.3, 56.3, 35.8, 34.6, 32.1, 31.3, 29.9, 29.9, 29.8, 29.7, 29.6, 29.6, 29.5, 28.0, 26.8, 25.2, 22.9, 14.3; MS (EI) m/z 342 (M-CO₂)+, 117, 104 (100), 91; HRMS (ESI) calcd for C₂₆H₄₃O₂ [M+H]⁺ m/z 387.3258, found 387.3259.

### Example 11: (trans)-3-Hexyl-2-methylene-4-tridecyloxetane

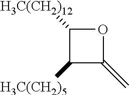

(trans)-3-Hexyl-4-tridecyloxetan-2-one (Example 7) (12 mg, 34 mmol) and Petasis solution (0.2 mL, 0.72 M) were stirred in a pre-heated oil bath at 80° C. for 6 h. See Dollinger et al. J. Org. Chem. (1996) 61, 7248. TLC showed remaining starting material so 0.2 mL more of the Petasis solution was added, and stirring was continued for 2 h. Petroleum ether (10 mL) was added to quench the reaction, and the mixture was allowed to stir overnight. The mixture was then filtered through a pad of celite until the filtrate was colorless, and the filtrate was concentrated to 1 mL solution. Purification by flash column chromatography on silica gel (petroleum ether/EtOAc/Et₃N, 96:3:1) gave (trans)-3-hexyl-2-methylene-4-tridecyloxetane (4.5 mg, 38%) as a slightly yellow oil: IR (neat) 2924, 2854, 1689, 784 cm⁻¹; ¹H NMR (400 MHz, CDCl₃) δ 4.39 (m, 1H), 4.06 (br s, 1H), 3.72 (br s, 1H), 2.96-2.91 (m, 1H), 1.85-1.78 (m, 1H), 1.71-1.62 (m, 3H), 1.29-1.26 (m, 30H), 0.89-0.86 (m, 6H); ¹³C NMR (100 MHz, CDCl₃) δ 167.9, 86.3, 78.5, 47.5, 36.0, 32.3, 32.2, 31.9, 29.9, 29.9, 29.7, 29.7, 29.6, 29.4, 27.0, 24.7, 22.9, 22.8, 14.3, 14.3; HRMS (ESI) calcd for C₂₃H₄₅O [M+H]⁺ m/z 337.3465, found 337.3469.

### Example 12: (trans)-2-Methylene-3-(5-phenylpentyl)-4-tridecyloxetane

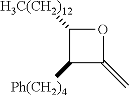

(trans)-3-(5-Phenylpentyl)-4-tridecyloxetan-2-one (Example 9), (11 mg, 27 mmol) and Petasis solution (0.080 mL, 0.50 M) were stirred in a pre-heated oil bath at 80° C. for 5 h. Petroleum ether (10 mL) was added to quench the reaction, and the mixture was allowed to stir overnight. The mixture was then filtered through a pad of celite, and the filtrate was concentrated to 1 mL solution. Purification by flash column chromatography on silica gel (petroleum ether/EtOAc/Et₃N 96:3:1) gave (trans)-2-methylene-3-(5-phenylpentyl)-4-tridecyloxetane (3.2 mg, 40%) as a colorless oil: IR (neat) 2922, 2852, 1687, 1454 cm⁻¹; ¹H NMR (500 MHz, CDCl₃) δ 7.30-7.26 (m, 2H), 7.19-7.16 (m, 3H), 4.38 (m, 1H), 4.06 (m, 1H), 3.71 (m, 1H), 2.94-2.90 (m, 1H), 2.64 (t, J=7.5 Hz, 2H), 1.84-1.76 (m, 1H), 1.70-1.62 (m, 5H), 1.36-1.26 (m, 26H), 0.91 (t, J=7.0 Hz, 3H); ¹³C NMR (125 MHz, CDCl₃) δ 167.8, 142.8, 128.6, 128.5, 125.9, 86.2, 78.5, 47.5, 36.1, 36.0, 32.2, 32.1, 31.5, 29.9, 29.9, 29.8, 29.7, 29.6, 29.4, 27.0, 24.7, 22.9, 14.3; MS (EI) m/z 398 (M+), 104 (100), 91; HRMS (ESI) calcd for C₂₈H₄₇O [M+H]⁺ m/z 399.3621, found 399.3594.

### Example 13: (E/Z)-6-(2-Oxo-4-tridecyloxetan-3-ylidene)hexanamide

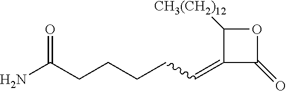

Catalyst B (0.03 g, 0.04 mmol) was added to a solution of 3-methylene-4-tridecyloxetan-2-one (Intermediate 3) (0.20 g, 0.75 mmol) and hept-6-enamide (Intermediate 5) (0.19 g, 1.5 mmol) in dry CH₂Cl₂ (29 mL). The mixture was stirred at 40° C. for 48 h. After cooling to rt, the CH₂Cl₂ was removed under reduced pressure to yield a greenish brown residue. Purification by flash chromatography on silica gel (CH₂Cl₂/MeOH, 98:2) gave (E/Z)-6-(2-oxo-4-tridecyloxetan-3-ylidene)hexanamide (Z/E, 3.7:1), (0.20 g, 73%) as an off white solid.

The Z-isomer was separable from the E-isomer by careful column chromatography. The E-isomer was not obtained as a single compound, but chemical shifts for a number of the protons and carbons were identifiable in their respective NMRs: E-isomer (13a): ¹H NMR (400 MHz, CDCl₃) δ 6.31 (t, J=7.5 Hz, 1H), 5.35 (br s, 2H) 5.00 (m, 1H), 2.28-2.23 (m, 2H), 2.16 (dt, J=7.4, 7.4 Hz, 2H), the remaining proton signals cannot be readily distinguished from those of the Z-isomer; ¹³C NMR (100 MHz, CDCl₃) δ 174.6, 164.4, 138.3, 133.2, 79.4, 35.5, 33.5, 32.1, 29.9, 29.8, 29.7, 29.7, 29.6, 29.6, 29.5, 28.9, 28.2, 28.1, 25.1, 25.0, 24.8, 24.6, 22.9, 14.3. Z-isomer (13b): mp: 106-107° C.; IR (neat) 3384, 3196, 2915, 2850, 1792, 1656, 1468, 1422, 1180, 1124, 1074, 803 cm⁻; ¹H NMR (500 MHz, CDCl₃) δ 5.86 (t, J=8.0 Hz, 1H), 5.51 (br s, 1H), 5.27 (br s, 1H), 4.87 (dd, J=6.5, 6.5 Hz, 1H), 2.59-2.47 (m, 2H), 2.26 (t, J=7.5 Hz, 2H), 1.82-1.77 (m, 2H), 1.73-1.67 (m, 2H), 1.58-1.52 (m, 2H), 1.47-1.42 (m, 2H), 1.37-1.26 (m, 20H), 0.88 (t, J=6.8 Hz, 3H); ¹³C NMR (125 MHz, CDCl₃) δ 175.0, 164.5, 136.2, 135.5, 79.0, 35.8, 33.9, 32.1, 29.8, 29.8, 29.8, 29.8, 29.7, 29.6, 29.5, 29.4, 28.2, 28.0, 24.8, 24.5, 22.8, 14.3; HRMS (ESI) calcd for C₂₂H₄₀NO₃ [M+H]+m/z 366.3003, found 366.3009.

Catalyst B is

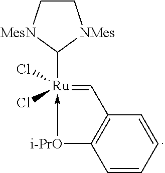

### Example 14: (E/Z)-6-(2-Oxo-4-(2-phenethyl)oxetan-3-ylidene)hexanamide

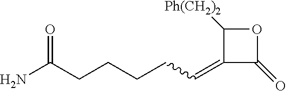

Catalyst B (0.026 g, 0.041 mmol) was added to a solution of 3-methylene-4-(2-phenethyl)oxetan-2-one (0.073 g, 0.41 mmol) and hept-6-enamide (Intermediate H) (0.053 g, 0.41 mmol) in dry CH₂Cl₂ (16 mL). The solution was stirred at 40° C. for 24 h. After the reaction was allowed to cool to rt, the CH₂Cl₂ was removed under reduced pressure to yield a greenish brown residue. Purification by flash chromatography on silica gel (CH₂Cl₂/MeOH, 99:1) gave (E/Z)-6-(2-oxo-4-(2-phenethyl)oxetan-3-ylidene)hexanamide (Z/E, 4:1) (0.050 g, 43%) as an off white solid: IR (neat) 3384, 3182, 2942, 1783, 1644, 1416, 1218, 1179, 1135, 1096, 1014 cm⁻¹; Peak assignment for Z-isomer (14b): ¹H NMR (400 MHz, CDCl₃) δ 7.32-7.28 (m, 2H), 7.23-7.18 (m, 3H), 5.74 (dt, J=8.0, 1.2 Hz, 1H), 5.35 (br s, 2H) 4.87 (dd, J=6.1, 6.1 Hz, 1H), 2.89-2.70 (m, 2H), 2.56-2.42 (m, 2H), 2.27-2.21 (m, 2H), 2.15-2.08 (m, 2H), 1.70-1.62 (m, 2H), 1.54-1.47 (m, 2H); ¹³C NMR (100 MHz, CDCl₃) δ 175.2, 164.3, 140.5, 137.8, 136.1, 128.8, 128.6, 126.55, 78.1, 35.6, 35.1, 31.1, 28.3, 28.1, 24.6; The E-isomer (14a): ¹H NMR (400 MHz, CDCl₃) δ 7.32-7.28 (m, 2H), 7.23-7.18 (m, 3H), 6.32 (dt, J=7.8, 1.6 Hz, 1H), 5.49 (br s, 2H) 4.99 (dd, J=8.0, 0.0 Hz, 1H), the remaining proton signals cannot be readily distinguished from those of the Z-isomer; ¹³C NMR (100 MHz, CDCl₃) δ 174.9, 164.2, 140.6, 138.0, 136.3, 133.7, 128.8, 128.6, 126.6, 78.2, 35.4, 35.3, 28.8, 28.1, 25.0; HRMS (ESI) calcd for C₁₇H₂₂NO₃ [M+H]+m/z 288.1594, found 288.1630.

### Example 15: (Z)-6-(2-Oxo-4-(9-phenylnonyl)oxetan-3-ylidene)hexanamide

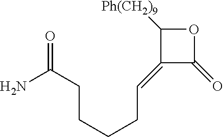

Catalyst A (0.025 g, 0.30 mmol) was added to a solution of 3-methylene-4-(9-phenylnonyl)oxetan-2-one (Intermediate 4) (0.085 g, 0.30 mmol) and hept-6-enamide (Intermediate 5) (0.038 g, 0.30 mmol) in dry CH₂Cl₂ (11 mL). The solution was stirred at 40° C. for 3 d. After the reaction was allowed to cool to rt, the CH₂Cl₂ was removed under reduced pressure. Purification of the residue by flash chromatography on silica gel (CH₂Cl₂/MeOH, 98:2) gave (E/Z)-6-(2-oxo-4-(9-phenylnonyl)oxetan-3-ylidene)hexanamide (Z/E, 3.7:1), (0.068 g, 59%) as an off white solid. Further chromatography using Et₂O gave pure Z-isomer: mp: 93-94° C.; IR (neat) 3384, 3194, 2916, 2850, 1791, 1655, 1467, 1421, 1180, 1124, 1074 cm⁻; 1H NMR (400 MHz, CDCl₃) δ 7.29-7.25 (m, 2H), 7.18-7.15 (m, 3H), 5.84 (t, J=8.0 Hz, 1H), 5.50 (s, 1H), 5.29 (s, 1H), 4.86 (dd, J=6.2, 6.2 Hz, 1H), 2.60 (t, J=7.8 Hz, 2H), 2.56-2.45 (m, 2H), 2.26 (t, J=7.3 Hz, 2H), 1.84-1.77 (m, 2H), 1.73-1.66 (m, 2H), 1.62-1.53 (m, 5H), 1.49-1.41 (m, 2H), 1.30-1.29 (m, 10H), 0.87 (t, J=6.7 Hz, 3H); ¹³C NMR (100 MHz, CDCl₃) δ 175.0, 164.6, 143.1, 138.3, 135.6, 128.6, 128.4, 125.8, 79.1, 36.2, 35.1, 33.9, 31.7, 29.6, 29.6, 29.6, 29.5, 29.5, 28.3, 28.1, 24.8, 24.6; HRMS (ESI) calcd for C₂₂H₄₀NO₃ [M+H]⁺ m/z 386.2690, found 386.2699.

### Example 16: (Z)-6-(2-Oxo-4-tridecyloxetan-3-ylidene)hexane-1-sulfonamide

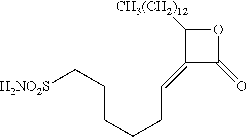

Catalyst B (0.02 g, 0.03 mmol) was added to a solution of 3-methylene-4-tridecyloxetan-2-one (Intermediate 3) (0.16 g, 0.60 mmol) and hept-6-ene-1-sulfonamide (Intermediate 6) (0.15 g, 0.90 mmol) in dry CH₂Cl₂ (23 mL). The solution was stirred at 40° C. for 48 h. After the reaction was allowed to cool to rt, the CH₂Cl₂ was removed under reduced pressure to yield a greenish brown residue. Purification by flash chromatography on silica gel (CH₂Cl₂/MeOH, 98:2) gave (Z/E)-6-(2-oxo-4-tridecyloxetan-3-ylidene)hexane-1-sulfonamide (Z/E, 4:1) (0.19 g, 79%) as an off white solid. The Z-isomer, isolated as a white solid, was separable from the E-isomer by careful chromatography using the same solvent system: mp: 79-80° C.; IR (neat) 2914, 2849, 1791, 1469, 1331, 1143 cm⁻; 1H NMR (400 MHz, CDCl₃) δ 5.84 (t, J=7.6 Hz, 1H), 4.92 (s, 2H), 4.86 (dd, J=6.2, 6.2 Hz, 1H), 3.11 (t, J=7.3 Hz, 2H), 2.58-2.24 (m, 2H), 1.91-1.85 (m, 2H), 1.82-1.77 (m, 2H), 1.51 (m, 4H), 1.45-1.39 (m, 2H), 1.34-1.25 (m, 20H), 0.87 (t, J=6.7 Hz, 3H); ¹³C NMR (100 MHz, CDCl₃) δ 164.6, 138.2, 135.6, 79.1, 55.1, 33.9, 32.1, 29.8, 29.8, 29.7, 29.6, 29.5, 29.5, 28.3, 28.2, 27.4, 24.8, 23.6, 22.9, 14.3; HRMS (ESI) calcd for C₂₂H₄₂NO₃ [M+H]⁺ m/z 416.2829, found 416.2842.

### Example 17: (cis/trans)-6-(2-Oxo-4-tridecyloxetan-3-yl)hexanamide

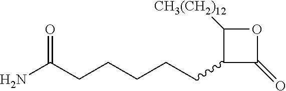

(Z)-6-(2-Oxo-4-tridecyl-oxetan-3-ylidene)hexanamide (Example 13b) (0.050 g, 0.14 mmol) was dissolved in mixture of THF:MeOH (0.9:0.2 mL). This solution was cooled to −10° C., followed by the addition of CoCl₂(PPh₃) (0.01 g, 0.04 mmol) and then portion-wise addition of NaBH₄ (13 mg, 0.41 mmol) within 10 min. The mixture was vigorously stirred for 4 h between −10 to −7° C. The reaction mixture was filtered through a pad of celite, and the celite was then washed with CHCl₃ (10 mL). The filtrate was washed with 2M HCl (5 mL), dried (MgSO₄) and concentrated. Purification by flash column chromatography on silica gel (CH₂Cl₂/MeOH, 98:2) gave an inseparable mixture of (cis/trans)-6-(2-oxo-4-tridecyloxe-tan-3-yl)hexanamide (trans/cis, 1.4:1) (18 mg, 35%) as an off white solid. Peak assignments for trans-isomer (17b): ¹H NMR (400 MHz, CDCl₃) δ 5.34 (br, 2H), 4.21 (ddd, J=6.2, 6.2, 4.0 Hz, 1H), 3.16 (ddd, J=7.8, 7.8, 4.0 Hz, 1H),), 2.23 (t, J=7.4 Hz, 2H), 1.87-1.81 (m, 2H), 1.78-1.65 (m, 5H), 1.55-1.26 (m, 27H), 0.88 (t, J=7.0 Hz, 3H); ¹³C NMR (100 MHz, CDCl₃) δ 175.2, 171.8, 78.3, 56.3, 35.7, 34.6, 32.1, 29.9, 29.9, 29.8, 29.7, 29.6, 29.6, 29.5, 29.0, 27.9, 26.8, 26.8, 25.2, 22.9, 14.3; (cis/trans mixture): HRMS (ESI) calcd for C₂₂H₄₂NO₃ [M+H]⁺ m/z 368.3159, found 368.3186.

Stereoselective synthesis of the cis-isomer (17a): (Z)-6-(2-Oxo-4-tridecyloxetan-3-yli-dene)hexanamide (Intermediate 13b) (50.0 mg, 0.14 mmol) was dissolved in THF (2 mL). Pd/C (10 mol %, 4 mg, 0.004 mmol) was added to the solution. The mixture was purged with H₂ gas for 5 min. It was then stirred under H₂ gas for 2 h. The mixture was filtered through a pad of celite, and the celite was washed with THF (5 mL). The filtrate was concentrated and purified by flash column chromatography on silica gel (CH₂Cl₂/MeOH, 98:2) to give (cis/trans)-6-(2-oxo-4-tridecyloxetan-3-yl)hexanamide (trans/cis, 1:9), (16 mg, 65%) as an off white solid. Careful chromatography under the same conditions provided the cis-isomer in >95% purity: mp 98-101° C.; IR (neat) 3393, 2918, 2849, 1795, 1649, 1417, 1132, 804 cm⁻¹; ¹H NMR (400 MHz, CDCl₃) δ 5.40-5.35 (br, 2H), 4.54-4.50 (m, 1H), 3.61-3.55 (m, 1H), 2.23 (t, J=6.1 Hz, 2H), 1.79-1.51 (m, 8H), 1.40-1.26 (m, 24H), 0.89-0.86 (m, 3H); ¹³C NMR (100 MHz, CDCl₃) δ 175.2, 172.5, 75.9, 52.7, 35.8, 32.1, 30.4, 29.9, 29.8, 29.7, 29.6, 29.6, 29.5, 29.0, 27.4, 25.8, 25.2, 23.9, 22.9, 14.3; MS (EI) m/z 323, 306, 294, 281, 264, 240, 226, 207, 168, 154, 140, 126, 112, 95, 81, 72, 59; HRMS (ESI) calcd for C₂₂H₄₂NO₃ [M+H]⁺ m/z 368.3159, found 368.3145.

### Example 18: (cis/trans)-6-(2-Oxo-4-(2-phenethyl)oxetan-3-yl)hexanamide

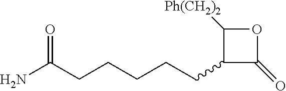

(Z/E)-6-(2-Oxo-4-(2-phenethyl)oxetan-3-ylidene)hexanamide (Example 14) (0.031 g, 0.011 mmol) was dissolved in THF (1 mL). Pd/C (10 mol %, 1.2 mg, 0.0011 mmol) was added to the solution. The mixture was purged with H₂ gas for 5 min. It was then stirred under H₂ gas for 2 h 15 min. The mixture was filtered through a pad of celite, and the celite was washed with THF (5 mL). The filtrate was concentrated and purified by flash column chromatography on silica gel (CH₂Cl₂/MeOH 98:1) to give (cis/trans)-6-(2-oxo-4-(2-phenethyl)oxetan-3-yl)hexanamide (trans/cis, 1:5,) (19 mg, 60%) as an inseparable mixture and off white solid. IR (neat) 3392, 2938, 1796, 1646, 1416, 1135, 836 cm⁻¹; cis-isomer (18a): ¹H NMR (400 MHz, CDCl₃) δ 7.33-7.29 (m, 2H), 7.24-7.19 (m, 3H), 5.58-5.50 (br, 2H), 4.54 (ddd, J=10.0, 6.4, 3.4 Hz, 1H), 3.59 (ddd, J=9.2, 6.5, 6.5 Hz, 1H), 2.88 (ddd, J=14.0, 9.4, 5.0 Hz, 1H), 2.73-2.66 (m, 1H), 2.21 (t, J=7.4 Hz, 2H), 2.10-2.01 (m, 1H), 1.98-1.78 (m, 1H), 1.83-1.90 (m, 1H), 1.67-1.50 (m, 4H), 1.43-1.32 (m, 3H); ¹³C NMR (100 MHz, CDCl₃) δ 175.4, 172.2, 140.6, 128.8, 128.7, 126.6, 74.7, 52.6, 35.7, 32.4, 29.9, 27.3, 25.1, 23.9; trans-isomer (18b): ¹H NMR (400 MHz, CDCl₃) δ 4.54 (ddd, J=10.0, 6.4, 3.4 Hz, 1H), 3.59 (ddd, J=9.2, 6.5, 6.5 Hz, 1H), the remaining proton signals cannot be readily distinguished from those of the cis-isomer; ¹³C NMR (100 MHz, CDCl₃) δ 175.4, 172.2, 140.6, 128.8, 128.7, 126.6, 74.7, 52.6, 35.7, 32.4, 29.9, 27.3, 25.1, 23.9; HRMS (ESI) calcd for C₁₇H₂₄NO₃ [M+H]+m/z 290.1751, found 290.1754.

### Example 19: (Z)-2-(6-(dimethylamino)-3-(dimethyliminio)-3H-xanthen-9-yl)-5-((5-(5-(2-oxo-4-tridecyloxetan-3-ylidene)pentanamido)pentyl)carbamoyl)benzoate (WHP01)

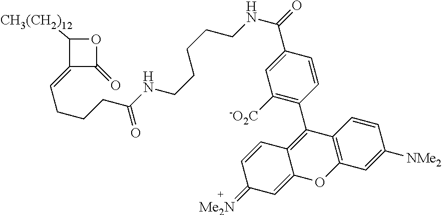

Step a. Preparation of (Z)-5-(2-oxo-4-tridecyloxetan-3-ylidene)pentanoic acid Catalyst A (8.5 mg, 0.01 mmol, 0.05 equiv) was added to a solution of 3-methylene-4-tridecyloxetan-2-one (Intermediate 3) (53 mg, 0.2 mmol, 1.0 equiv) and 6-hexenoic acid (36 μL, 0.3 mmol, 1.5 equiv) in dry CH₂Cl₂ (6 mL). The pale brown solution was then stirred at 40° C. for 48 h. The solution was concentrated under reduced pressure to give a brown residue, which was purified by column chromatography on silica gel (35% EtOAc in hexanes+1% AcOH) to provide the title compound as a white powder (Z:E=4:1), (28 mg, 40%). The Z-isomer was separable from the E-isomer by careful chromatography. ¹H NMR (400 MHz, CDCl₃) δ 5.84 (td, J=8.0, 1.2 Hz, 1H), 4.86 (dd, J=6.4, 6.4 Hz, 1H), 2.61-2.51 (m, 2H), 2.41 (t, J=7.0 Hz, 2H), 1.86 1.77 (m, 4H), 1.48-1.26 (m, 22H), 0.88 (t, J=6.8 Hz, 3H); ¹³C NMR (125 MHz, CDCl₃) δ 178.5, 164.1, 138.8, 134.4, 79.0, 33.8, 33.2, 32.1, 29.8, 29.8, 29.8, 29.6, 29.6, 29.5, 29.4, 28.2, 24.8, 23.9, 22.8, 14.3; HRMS (ESI-TOF⁺) calcd for C₂₁H₃₆O₄[M+H]⁺ m/z 353.2686, found 353.2689.

### Step b. Preparation of 2,5-dioxopyrrolidin-1-yl (Z)-5-(2-oxo-4-tridecyloxetan-3-ylidene)pentanoate

To a solution of (Z)-5-(2-oxo-4-tridecyloxetan-3-ylidene)pentanoic acid (28 mg, 0.08 mmol, 1.0 equiv) and DIPEA (21 μL, 0.12 mmol, 1.5 equiv) in dry CH₂Cl₂ (1 mL) was added N,N-disuccinimidyl carbonate (23 mg, 0.09 mmol, 1.1 equiv). The reaction mixture was stirred at rt for 3 h, eventually forming a white suspension. The mixture was concentrated under reduced pressure and purified by column chromatography on silica gel (1:2:2 EtOAc/hexanes/CH₂Cl₂) to provide the title compound (29 mg, 80%) as a white powder. ¹H NMR (400 MHz, CDCl₃) δ 5.85 (td, J=7.6, 0.8 Hz, 1H), 4.88 (dd, J=6.4, 6.4 Hz, 1H), 2.84 (s, 4H), 2.67 (t, J=7.6 Hz, 2H), 2.67-2.54 (m, 2H), 1.98-1.91 (m, 2H), 1.84-1.78 (m, 2H), 1.50-1.39 (m, 2H), 1.36-1.26 (m, 20H), 0.88 (t, J=6.8 Hz, 3H); ¹³C NMR (125 MHz, CDCl₃) δ 169.1, 168.2, 164.0, 139.3, 133.6, 79.0, 33.8, 32.1, 30.4, 29.8, 29.8, 29.8, 29.6, 29.5, 29.5, 29.4, 28.0, 25.7, 24.8, 23.8, 22.8, 14.3; HRMS (ESI-TOF⁺) calcd for C₂₅H₃₉NO₆ [M+H]⁺ m/z 450.2850, found 450.2851.

### Step c. Preparation of (Z)-2-(6-(dimethylamino)-3-(dimethyliminio)-3H-xanthen-9-yl)-5-((5-(5-(2-oxo-4-tridecyloxetan-3-ylidene)pentanamido)pentyl)carbamoyl)benzoate (WHP01)

2,5-Dioxopyrrolidin-1-yl (Z)-5-(2-oxo-4-tridecyloxetan-3-ylidene)pentanoate (4.5 mg, 0.01 mmol, 1.0 equiv), dissolved in dry CH₂Cl₂ (0.5 mL), was slowly added to a solution of 5-TAMRA cadaverine (5.0 mg, 0.01 mmol, 1.0 equiv) and DIPEA (9 μL, 0.05 mmol, 5.0 equiv) in dry CH₂Cl₂ (0.5 mL), chilled in an ice bath. The dark pink solution was allowed to slowly warm to rt to stir for 2 h. The reaction mixture was then concentrated under reduced pressure, and the residue purified by column chromatography on silica gel (10% to 15% MeOH/CH₂Cl₂) to yield the title compound (4.6 mg, 54%) as a dark pink solid. ¹H NMR (500 MHz, CDCl₃) δ 8.39 (s, —NH), 8.19 (d, J=8.4 Hz, 1H), 7.12 (d, J=7.2 Hz, 1H), 6.85-6.80 (m, 1H), 6.57 (d, J=8.8 Hz, 2H), 6.49 (s, 2H), 6.39 (d, J=8.8 Hz, 2H), 5.86 (t, J=7.6 Hz, 1H), 5.12 (s, —NH), 4.83 (dd, J=6.4, 6.4 Hz, 1H), 3.54-3.49 (m, 2H), 3.32-3.27 (m, 2H), 2.99 (s, 12H), 2.57-2.43 (m, 2H), 2.22 (t, J=6.8 Hz, 2H), 1.86-1.55 (m, 8H), 1.49-1.39 (m, 4H), 1.31-1.25 (m, 20H), 0.88 (t, J=6.4 Hz, 3H) ppm; HRMS (ESI-TOF⁺) calcd for C₅₁H₆₈N₄O₇ [M+H]⁺ m/z 849.5161, found 849.5164.

### II. Biological Evaluation

All PS-lipase substrate assays were performed on either 18:0-18:2 PS (Avanti Catalog #840063) or 16:0-18:1 PS (Avanti Catalog #840034). 17:1 FFA (Sigma-Aldrich #H8896) was used an internal standard for substrate assays, as well as lipidomic analysis. 2-AG-d5 (Catalog #362162) and PGE2-d9 (Catalog #10581) were purchased from Cayman Chemicals. COLO205, MCF7, K562 and HEK293T cells were purchased from ATCC. All full-length cDNA constructs coding for serine hydrolases ABHD16A, and ABHD12 were purchased from Open Biosystems (GE Healthcare), and all primers were obtained from Integrated DNA Technologies. All DNA sequencing was performed by Eton Biosciences Inc. All cytokine analysis was done using single analyte ELISA kits purchased from R&D Systems, Mouse TNF-α (Catalog # DY410), Mouse IL-6 (Catalog # DY406), and Mouse IL-1β (Catalog # DY401). All tissue culture media and supplements (RPMI 1640, DMEM, and Trypsin) were purchased from Caisson Labs, and SILAC RPMI was purchased from Thermo Scientific. Phenol red free RPMI 1640 media was purchased from Life Technologies (Catalog #1835-030). All mouse studies were done using the C57Bl/6 mouse line unless stated otherwise.

Mass spectrometry was performed using a ThermoFinnigan LTQ (for spectral counting studies), a ThermoFinnigan LTQ-Orbitrap (for SILAC studies), or a ThermoFinnigan Orbitrap Velos (for ReDiMe studies) following previously described protocols. See Jessani et al. Nat. Methods (2005) 2, 691-697; Hsu et al. Nat. Chem. Bio. (2012) 8, 999-1007. Peptides were eluted using a five-step multidimensional LC-MS protocol in which increasing concentrations of ammonium acetate are injected followed by followed by a gradient of increasing acetonitrile, as previously described. See Jessani et al. Nat. Methods (2005) 2, 691-697; Washburn et al. Nat. Biotechnol. (2001) 19, 242-247. For all samples, data were collected in data-dependent acquisition mode over a range from 400-1800 m/z. Each full scan was followed by up to 7- or 30-fragmentation events for experiments utilizing the LTQ and Orbitrap or Orbitrap Velos instruments, respectively. Dynamic exclusion was enabled (repeat count of 1, exclusion duration of 20 s) for all experiments. The data were searched using the ProLuCID algorithm (http://fields.scripps.edu), against a mouse or human reverse-concatenated nonredundant (gene-centric) FASTA database that was assembled from the Uniprot database (http://www.uniprot.org/). ProLuCID searches specified static modification of cysteine residues (+57.0215 m/z; iodoacetamide alkylation) and required peptides to contain at least one tryptic terminus. For ReDiMe samples, each data set was independently searched with light and heavy parameter files; for the light search, static modifications on lysine (+28.0313 m/z) and N-termini (+28.0313 m/z) were specified; for the heavy search, static modifications on lysine (+34.06312 m/z) and N-termini (+34.06312 m/z) were specified. For SILAC samples, datasets were searched independently with the following parameter files; for the light search, all amino acids were left at default masses; for the heavy search, static modifications on lysine (+8.0142 m/z) and arginine (+10.0082 m/z) were specified. For data collected on the Orbitrap mass spectrometers, precursor-ion mass tolerance was set to 50 ppm. The resulting peptide spectral matches were filtered using DTASelect (version 2.0.47), and only half-tryptic or fully tryptic peptides were accepted for identification. Peptides were restricted to a specified false positive rate of <1%. SILAC and ReDiMe ratios were quantified using in-house CIMAGE software12. Briefly, a 10 minute retention time window was used for peak identification using 10 ppm mass accuracy and requiring a co-elution R2 value greater than 0.8. Peptides detected as singletons, where only the heavy or light isotopically labeled peptide was detected and sequenced, but which passed all other filtering parameters, were given a ratio of 20, which is the maximum SILAC or ReDiMe ratio reported here.

All statistical analysis was performed using the GraphPad Prism 6 (for Mac OS X) software. All data is shown as mean±s.e.m. Student's t-test (two-tailed) was used to study statistically significant differences between study groups. A p-value of <0.05 was considered statistically significant for this study.

### Example 20: Competitive Gel-Based ABPP Analysis

Competitive gel-based ABPP were performed as described previously with slight modifications. See Adibekian et al. Nat. Chem. Biol. (2011) 7, 469. Briefly the tissue (mouse brain) and cell (COLO205 cancer cell line) membrane proteomes were adjusted to a final concentration 1 mg/mL in a reaction volume of 100 μL and treated with the β-lactone compounds at a final concentration of 25 μM for 30 minutes at 37° C. with constant shaking. Thereafter 2 μM FP-rhodamine was added to label the proteins to assess their activities in gel for 30 minutes at 37° C. with constant shaking. Proteomes were also treated with DMSO and tetrahydrolipstatin (25 μM) as controls. The reactions were quenched by adding 40 μL of 4×SDS-PAGE loading buffer for reducing gels, followed by boiling for 2 minutes. All samples were visualized in-gel using an FMBio II Multiview flatbed fluorescence scanner (Hitachi). The fluorescence from rhodamine is presented in gray scale.

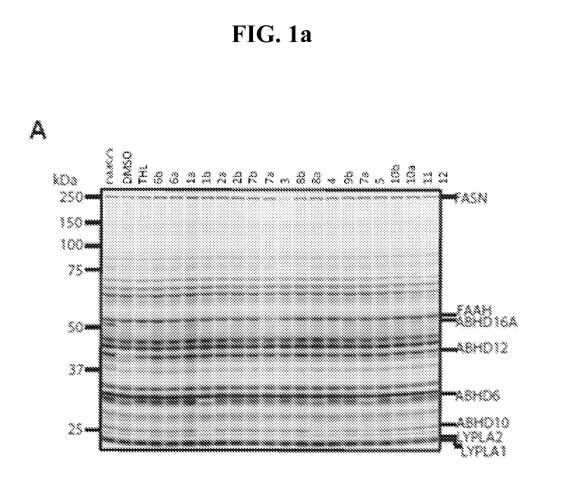

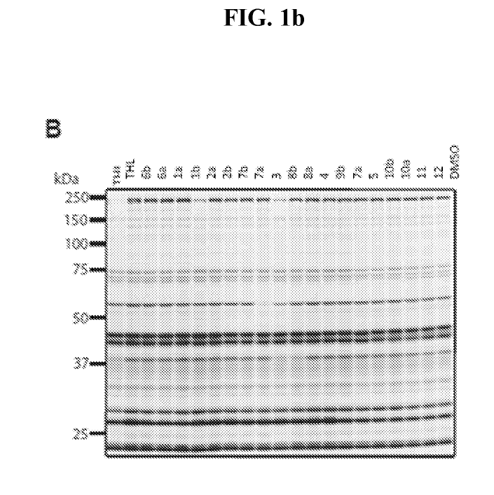

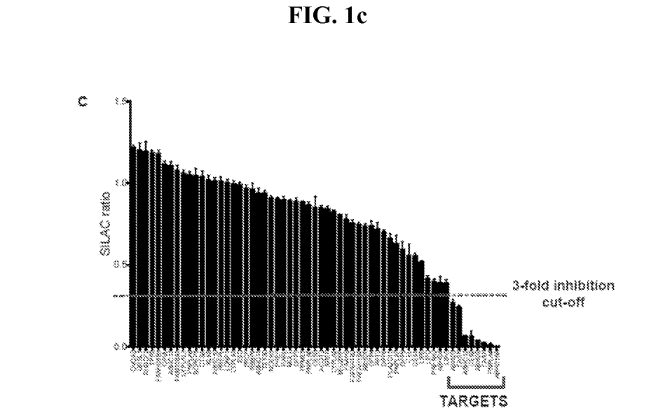

Results from these studies are shown in FIGS. 1a and 1b. The β-lactone compounds inhibited activities of several serine hydrolase targets in the mouse brain membrane proteome (FIG. 1a), including fatty acid synthase (FASN), lysophospholipase 1 and 2 (LYPLA1, LYPLA2), ABHD10, and ABHD16A. Profiles of the human COLO205 colon cancer membrane proteome (FIG. 1b) also revealed a diverse set of serine hydrolase targets for the β-lactone compounds. Compound 8b exhibited the broadest reactivity across the detected serine hydrolases.

### Example 21: ABPP-SILAC Sample Preparation and Analysis

All ABPP-SILAC experiments were performed using the human COLO205 colon cancer cell line. Light and heavy amino acid-labeled COLO205 cancer cell lines were generated using standard SILAC culturing protocols, using SILAC RPMI media. 2 mg/mL light proteome (0.5 mL) and 2 mg/mL heavy proteome (0.5 mL) were treated separately with 25 μM KC6-10-3 and DMSO respectively for 30 minutes at 37° C., and subsequently labeled with FP-biotin (5 μM final concentration) for 45 minutes at 37° C. with constant shaking. After FP-biotin labeling, the proteomes were combined, denatured and precipitated using 4:1 MeOH: CHCl₃, resuspended in 0.5 mL of 6M urea in PBS, reduced using tris(2-carboxyethyl)-phosphine (TCEP) (10 mM final concentration) for 30 minutes at 37° C., and then alkylated using iodoacetamide (40 mM final concentration) for 30 minutes at 25° C. in the dark. The biotinylated proteins were enriched with PBS washed avidin-agarose beads (100 μL) (Sigma-Aldrich) by shaking at 25° C. for 1.5 hours in PBS with 2% SDS to final volume of 5.5 mL. After enrichment, the beads were separated from the unlabeled/unenriched proteome by centrifugation at 2800×g for 10 minutes and washed sequentially, first with 10 mL PBS with 0.2% SDS (×3 times), then with 10 mL PBS (×3 times) and finally with 10 mL DI H₂O (×3 times). On bead digestion was performed using sequence-grade trypsin (2 μg) (Promega Catalog # V5111) in 2M urea in PBS with 2 mM CaCl₂ for 12-14 hours at 37° C. Peptides obtained from this procedure were acidified using 5% (vol/vol) formic acid, and stored at −80° C. prior to analysis. Extensive probe-probe, probe-no probe experiments were done prior to testing compounds in the SILAC cell lines, to ensure complete labeling of heavy proteomes, and enrichment of SH targets by FP-biotin probe (data not shown). Peptide samples were analyzed using the multidimensional protein identification technology (MudPIT) that employs liquid chromatography with tandem mass spectrometry. See Washburn et al. Nat. Biotechnol. (2001) 3, 242; Jessani et al. Nat. Methods (2005) 2, 691. Briefly, peptides are eluted in a 5-step MudPIT experiment utilizing 0%, 25%, 50%, 80% and 100% salt bumps of ammonium acetate (aqueous), and the data was collected in data-dependent acquisition mode as described previously. See Hsu et al. Nat. Chem. Biol. (2012) 8, 999. The MS2 spectra were extracted from the raw file using the RAW Xtractor software (version 1.9.9.2). The MS2 spectra were searched using the ProLuCID algorithm against the latest non-redundant protein IDs for human or mouse proteins listed in Uniprot database. See The Uniprot Consortium, Nucl. Acids Res. (2014) 42, D191. ProLuCID has provisions for identification of static modification like alkylation of cysteine residues (+57.02146), methionine oxidation (+15.9949), and mass shifts from heavy amino acids in SILAC experiments (+10.0083 for arginine, and +8.0142 for lysine), enzyme specificity (half-tryptic specificity) and a precursor ion mass tolerance (50 ppm was used in this study). Lastly the MS2 spectra searches were subjected to filters using the DTASelect (version 2.0) for minimum number of peptides per protein, statistics with delta mass (-- mass), statistics with modifications (-- modstat), and statistics with tryptic status (--trypstat). The light/heavy peptide ratios for ABPP-SILAC experiments were computed using the in-house software CIMAGE. See Weerapana et al. Nature (2010) 468, 790. Reported ratios in this study represent the median of the unique quantified peptides per protein. Proteins with less than 2 quantified peptides were excluded from this study. The ABPP-SILAC experiments were conducted using an Agilent 1200-series quaternary pump, and a Thermo Scientific Finnigan series LTQ-Orbitrap ion trap mass spectrometer.

Enriched serine hydrolases that showed substantially greater (>3-fold) MS1 signals in DMSO (heavy) versus compound 8b (light)-treated proteomes were considered targets of the compound 8b probe (FIG. 1c). Eight serine hydrolases satisfied this criterion, with the most potently inhibited targets being ABHD16A, PNPLA4, FAAH, ABHD2, CES2, and ABHD12 (all inhibited >90%). A second set of enzymes that included FASN and ABHD3 were inhibited from 70-90%.

### Example 22: Generation of ABHD16A^{-/-} Mice

ABHD16A^{-/-} mice were generated on the C57Bl/6 background using the EUCOMM ES clone EPD0605_5_A11, which contains a gene trap-targeting cassette for the 13-galactosidase. This strain has conditional potential to generate tissue specific knockout mice through removal of the targeting cassette using Flp recombinase. Male ABHD16A^{+/-} mice were initially bred with female ABHD16A^{+/+} mice on the C57Bl/6 background to yield a pool of ABHD16A^{+/-} mice. The ABHD16A^{+/-} mice were further bred to eventually yield ABHD16A^{-/-} mice on the C57Bl/6 background. ABHD16A genotypes were confirmed using PCR amplification of the genomic tail DNA with the following primers: ABHD16A forward 5′-GGCCAGCCTGAGTTCCATAG-3′, ABHD16A reverse 5′-GGGCCTCTTAGGTGGGAAAC-3′, and ABHD16A gene trap reverse 5′-TCGTGGTATCGTTATGCGCC-3′. This PCR amplification strategy yielded a 536-bp product for the WT allele, and a 198-bp product for the gene-trap mutant allele. All mice used in this study were generated from breeding ABHD16A+/− mice, and had ad libitum access to water and food.

### Example 23: RT-PCR

Total RNA was isolated from half a brain of 10-week old ABHD16A^{+/+} and ABHD16A^{-/-} mice using RiboZol RNA extraction reagent (Amresco) as per manufacturer's instructions. Subsequently SuperScript III first-strand synthesis system (Life Technologies) was used to synthesize first strand cDNA from the total RNA isolated as per manufacturer's protocol. PCR amplification (50 cycles) of a 206-bp fragment of the ABHD16A cDNA was performed using 5′-TGGAAGCCACACATAGGAACC-3′ and 5′-CCTGTTGAGAAACGTGTCTGC-3′ primers. As controls PCR amplification (50 cycles) of a 211-bp fragment of the GAPDH cDNA using 5′-TGGATTTGGACGCATTGGTC-3′ and 5′-TTTGCACTGGTACGTGTTGAT-3′ primers, and a 154-bp fragment of the ACTB cDNA using 5′-GGCTGTATTCCCxCTCCATCG-3′ and 5′-CCAGTTGGTAACAATGCCATGT-3′ primers was performed. All primers were designed from the PrimerBank database from http://pga.mgh.harvard.edu/primerbank/.

### Example 24: Preparation of Mouse Tissue Proteomes

Mice were anaesthetized with isoflurane and then killed by cervical dislocation. Tissues (brains and spinal cords) were harvested for these mice. The tissue of interest (half-brain or entire spinal cord) were initially suspended in 0.5 mL of cold PBS in a 1.5 mL centrifuge tube and was homogenized using a Bullet Blender 24 (Next Advance) using 1 scoop of 0.5 mm diameter glass beads (Next Advance Catalog # GB05) at a speed setting of 8, for 3 minutes at 4° C. Thereafter 0.5 mL of cold PBS was added to the homogenates, and mixed by pipetting 8-10 times, and centrifuged at 1400×g for 5 minutes at 4° C., to separate tissue debris, and glass beads from the proteome. The supernatant (˜750 μL) was separated and centrifuged at 16,000×g for 45 minutes at 4° C. The supernatant obtained from this step was saved as the “soluble” proteome. The pellet obtained from this step was washed 3 times using 0.5 mL cold PBS, and thereafter re-suspended by pipetting in 750 μL of cold PBS. This proteome was saved as the “membrane” proteome. All protein concentrations were measured using Bio-Rad DC Protein Assay Kit as per manufacturer's protocol. 100 μL aliquots of both the proteome were stored at −80° C. for later use. Sonication to re-suspend the membrane proteome seems to impair the ABHD16A activity for reasons not fully understood.

### Example 25: Recombinant Expression of ABHD16A

HEK293T cells were grown to 40% confluence in a 15 cm dish in 25 mL of Dulbecco's modified Eagle medium (DMEM) supplemented with L-glutamine and 10% (vol/vol) FCS at 37° C. and 5% CO₂ (vol/vol). The cells were transiently transfected with 12 μg of the full-length cDNA of human ABHD16A (Open Biosystems Catalog # MHS6278-202758315) or murine ABHD16A (Open Biosystems Catalog # MMM1013-202760516) in pCMV-SPORT6 vector using 40 μg polyethyleneimine “MAX” (MW 40,000) (PEI) (Polysciences Inc. Catalog #24765) as the transfection reagent as per manufacturer's instructions. As a “mock” control, an empty vector was transiently transfected. 48 hours after transfection, the cells were harvested by scrapping, washed 3 times with PBS, and re-suspended in 1 mL of PBS. The cells were lysed by sonication, and 200 μL aliquots were flash frozen, and stored at −80° C. for further use. Protein concentration was measured using the Bio-Rad DC Protein Assay Kit as per manufacturer's protocol, and ABHD16A expression was confirmed using gel-based ABPP.

### Example 26: Substrate Hydrolysis Assays

All the lipid species used in the lipid substrate hydrolysis assays were purchased from Avanti Polar Lipids Inc unless mentioned otherwise. In general, 20 μg of proteome was incubated with 100 μM lipid in a reaction volume of 100 μL in PBS at 30° C. with constant shaking. After 30 minutes the reaction was quenched with 350 μL of 2:1 (vol/vol) CHCl₃: MeOH, doped with 0.5 nmol internal standard (17:1 FFA). The mixture was vortexed, and the centrifuged at 2800×g for 5 minutes to separate the aqueous (top) and organic (bottom) phase. The organic phase was collected. 5 μL of formic acid was added to the aqueous phase, and extracted again using 350 μL of 2:1 (vol/vol) CHCl₃: MeOH using above mentioned protocol. The organic phase was collected, and pooled with the organic phase from the first extraction. The pooled organic phase (˜700 μL) was then dried under a stream of N₂, re-solubilized in 150 μL of 2:1 (vol/vol) CHCl₃: MeOH, and subjected to MS analysis. A fraction of the organic extract (˜20 μL) was injected onto an Agilent 6520 quadrupole-time-of-flight (QTOF) LC-MS. LC separation was achieved using a Gemini 5U C-18 column (Phenomenex, 5 μm, 50×4.6 mm) coupled to a Gemini guard column (Phenomenex, 4×3 mm, Phenomenex security cartridge). The LC solvents were: buffer A: 95:5 (vol/vol) H₂O: MeOH+0.1% ammonium hydroxide, and buffer B: 60:35:5 (vol/vol) iPrOH:MeOH:H₂O+0.1% ammonium hydroxide. A typical LC run consisted of 15 minutes post-injection: 0.1 mL/min 100% buffer A from for 1.5 minutes, 0.5 mL/min linear gradient to 100% buffer B over 5 minutes, 0.5 mL/min 100% buffer B for 5.5 minutes, and equilibration with 0.5 mL/min 100% buffer A for 3 minutes. All MS analysis was performed using an electrospray ionization source (ESI) in negative ion mode for product formation (free fatty acid from lipid). The following parameters were used for the ESI-MS analysis: drying gas temperature: 350° C., drying gas flow rate: 11 mL/min, fragmentor voltage: 100 V, capillary voltage: 4 kV, and nebulizer pressure: 45 psi. All the data was collected and analyzed using the Agilent MassHunter Workstation software version B.04 using the Data Acquisition and Quantitative Analysis programs respectively. Measuring the area under the peak, and normalizing it to the internal standard quantified the product release for the lipid substrate hydrolysis assays. The substrate hydrolysis rate was corrected by subtracting the non-enzymatic rate of hydrolysis, which was obtained by using heat-denatured proteome (15 minutes at 95° C.) as a control. All the data presented for substrate hydrolysis assays is the average of three independent biological replicates, error bars represent s.e.m. Some products from the substrate hydrolysis assays were detected in positive ion mode for qualitative identification. All the MS parameters for the positive ion mode experiments, and LC separation conditions were the same except for the buffers, which contained 0.1% formic acid instead of 0.1% ammonium hydroxide. A comprehensive list of the substrates assayed against recombinant ABHD16A (mouse) is presented in Table 2.

### Example 27: ABPP-MudPIT and ABPP-SILAC Sample Preparation and Analysis

For the ABPP-MudPIT samples, proteomes were adjusted to a final concentration of 1 mg/mL in 1 mL of PBS, and labeled with FP-biotin (5 μM final concentration) for 45 minutes at 37° C. with constant shaking. After labeling, the proteomes were denatured and precipitated using 4:1 MeOH: CHCl₃, re-suspended in 0.5 mL of 6 M urea in PBS, reduced using tris(2-carboxyethyl)phosphine (TCEP) (10 mM final concentration) for 30 minutes at 37° C., and then alkylated using iodoacetamide (40 mM final concentration) for 30 minutes at 25° C. in the dark. The biotinylated proteins were enriched with PBS-washed avidin-agarose beads (100 μL) (Sigma-Aldrich) by shaking at 25° C. for 1.5 hours in PBS with 2% SDS to final volume of 5.5 mL. After enrichment, the beads were separated from the unlabeled/unenriched proteome by centrifugation at 2800×g for 10 minutes. The beads were washed sequentially, first with 10 mL PBS with 0.2% SDS (×3 times), then with 10 mL PBS (×3 times), and finally with 10 mL DI H₂O (×3 times). On-bead digestion was performed using sequence-grade trypsin (2 μg) (Promega Catalog # V5111) in 2M urea in PBS with 2 mM CaCl₂ for 12-14 hours at 37° C. Peptides obtained from this procedure were acidified using 5% (vol/vol) formic acid and stored at −80° C. prior to analysis. All SILAC experiments were performed using the human COLO205 colon cancer cell line. Isotopically light and heavy amino acid-labeled COLO205 cancer cell lines were generated using standard SILAC culturing protocols. For ABPP-SILAC experiments, 2 mg/mL light proteome (0.5 mL) and 2 mg/mL heavy proteome (0.5 mL) were treated with 10 μM final concentration of FP-biotin, combined, and thereafter processed similar to the ABPP-MudPIT protocol described above.

Extensive in vitro “probe-versus-probe” and “native-versus-heat-denatured proteome” comparisons were performed prior to using the SILAC lines to confirm the full incorporation of heavy-isotopic label (verified in probe-probe experiments where both heavy and light proteomes were treated with FP-biotin and the resulting SILAC peptide ratios for serine hydrolases generally centered around ˜1.0) and enrichment of active serine hydrolases (verified in native-versus-heat-denatured proteome experiments where light proteome was heat-denatured for 15 minutes at 95° C., and then both light and heavy proteomes were labeled and enriched using FP-biotin; only serine hydrolases showing greater than 5-fold enrichment in heavy proteomes were considered active and included in subsequent analyses).

### Example 28: ABPP-Reductive Dimethylation (ReDiMe)

Mouse half brain membrane proteomes and ABPP-MudPIT samples were prepared as described above, with minor adjustments. The final wash steps and trypsin digestion were performed in 100 mM triethylammonium bicarbonate buffer in preparation for downstream reductive dimethylation labeling. Reductive dimethylation was performed as previously described in the literature. See Boersema et al. Nature Protocols 4, 484-494; Wilson-Grady et al. Methods 61, 277-286. Briefly, either 4% ¹³C-labeled deuterated formaldehyde (heavy) or formaldehyde (light) was added to each sample (0.15% final concentration) followed by addition of 0.6 M sodium cyanoborohydride (22.2 mM final concentration). Following a 1 h incubation period at room temperature, the reaction was quenched by addition of 1% ammonium hydroxide (0.23% final concentration) and 5% formic acid (0.5% final concentration). The samples were then combined and analyzed by LC-MS analysis.

### Example 29: shRNA Knockdown Studies

The human K562 leukemia cancer cell line was chosen to stably knockdown ABHD16A, since this cell line had sufficient ABHD16A activity for detection by convenient gel-based ABPP assays. ABHD16A MISSION® shRNA bacterial glycerol stocks (Product type: SHCLNG-NM_021160) were purchased from Sigma-Aldrich, and the lentiviral-based shRNA gene knockdown was performed using standard manufacturers protocol. 6 shRNA transfer vector constructs were used initially to generate lentiviral transduction particles (Sigma-Aldrich catalog # TRCN0000046818, TRCN0000046819, TRCN0000046820, TRCN0000046821, TRCN0000046822, and TRCN0000413301). Briefly, 1 μg shRNA transfer vector, 1 μg of the pCMV-VSVG envelope vector, and 1 μg packaging vector, were transfected using 10 μg of PEI (transfection reagent), into 2.5×105 HEK293T cells cultured in 2 mL DMEM media with L-glutamine and 10% (vol/vol) FCS to generate the lentiviral transduction particles as per manufacturer's instructions (http://www.sigmaaldrich.com/life-science/functional-genomics-and-rai/shma/leaming-center/lentiviral-packaging.html). 1×10⁶ K562 cells were infected with the lentiviral transduction particles generated from the transfections along with 8 μg/mL polybrene (to enhance infection) cultured in 10 mL RPMI 1640 media with 10% FCS, and thereafter the lentiviral-infected K562 cells were selected on puromycin (2 μg/mL). After 6 rounds of selection on puromycin, the K562 cells infected with the lentiviral particles generated using the constructs TRCN0000046818 (KD_1) and TRCN0000413301 (KD_2) showed greater than 90% knockdown of ABHD16A by gel-based ABPP and substrate assays, and were selected for further studies. As a negative control, empty shRNA transfer vector was used (Sigma-Aldrich catalog # SHC001).

### Example 30: Sample Preparation and Targeted LC-MS Metabolite Profiling

ABHD16A^{+/+,} ABHD16A^{+/+}, and ABHD16A^{-/-} littermates of 10 weeks age were anaesthetized by isoflurane and killed by decapitation. The tissues (brains were sliced laterally into two hemispheres; entire spinal cord) were harvested, weighed, and immediately submerged in liquid N₂. Tissues (one half brain or entire spinal cord) were then Dounce-homogenized in 8 mL of 2:1:1 (vol/vol/vol) CHCl₃: MeOH: PBS containing the internal standard mix (1 nmol 17:1 FFA, and 100 pmol each of 17:1 lyso-PS, 17:0-20:4 PS, 17:1 lyso-PC, 17:0-20:4 PC, 17:0-20:4 PE and 50 pmol of 2-AG-d5). Homogenates were transferred to a glass vial and centrifuged at 2800×g for 10 minutes to separate the two phases. The organic phase (bottom) was removed, 200 μL of formic acid was added to acidify the aqueous homogenate (to enhance extraction of phospholipids), and CHCl₃ was added to make up 8 mL volume. The mixture was vortexed, and separated using centrifugation described above. Both the organic extracts were pooled, and dried under a stream of N₂. The metabolomes were then re-solubilized in 120 μL of 2:1 (vol/vol) CHCl₃: MeOH, and 10 μL were used for the targeted LC-MS analysis. For cellular and secreted metabolomics studies, serum free phenol red free RPMI 1640 media was used to prevent any promiscuous contributions from serum lipids. For the cellular metabolomics studies, 3×10⁶ cells were used for all cancer cell lines (COLO205, K562, and MCF7) and lymphoblast cell lines (LCLs), and 2×10⁶ cells were used for thioglycollate-elicited peritoneal macrophages. 4 mL of the serum free phenol red free RPMI 1640 media was harvested from all cultures to measure secreted metabolites (with the exception of the K562 leukemia cell line which need 10% FCS complementation for culturing and did not suit secreted metabolic studies protocol). For cellular metabolomics samples, the cells were washed with PBS (×3 times), and transferred into a glass vial using 1 mL PBS. 3 mL of 2:1 (vol/vol) CHCl₃: MeOH with the internal standard mix was added, and the mixture was vigorously vortexed. The two phases were separated by centrifugation at 2800×g for 10 minutes. The organic phase (bottom) was collected, and the aqueous phase was re-extracted after acidification by 50 μL of formic acid. The organic phases were pooled, and dried under a stream of N₂. The metabolome was re-solubilized using 120 μL of 2:1 (vol/vol) CHCl₃: MeOH, and 10 μL were used for the targeted LC-MS analysis. For the secreted metabolomic profiles, 4 mL of the serum free phenol red free RPMI 1640 media harvested from the cells, was transferred to a glass vial, to which 12 mL of 2:1 (vol/vol) CHCl₃: MeOH with the internal standard mix was added, and the mixture was vigorously vortexed. The two phases were separated by centrifugation at 2800×g for 10 minutes, the organic phase (bottom) was collected. The aqueous phase was re-extracted after acidification with 200 μL of formic acid. The organic phases were pooled, and dried using a stream of N₂. Thereafter the secreted metabolome was re-solubilized using 120 μL of 2:1 (vol/vol) CHCl₃: MeOH, and 20 μL were used for the targeted LC-MS analysis.

All the metabolites analyzed in this study were quantified using the multiple reaction monitoring (MRM) methods the targeted LC/MS analysis was done using an Agilent Technologies 6460 Triple Quad LC/M with an Agilent Technologies 1290 Infinity series quaternary pump. All data was collected using the LC/MS Data Acquisition mode of the Mass Hunter Workstation Software version B.05.00 build 5.0.5027.0 SP, and analyzed using the Quantitative Analysis mode of the same software. The solvents and LC separation conditions were the same as those described in the substrate assays section. All the MS analysis was performed using an electrospray ion source (ESI) using the following parameters: drying gas temperature=350° C., drying gas flow=9 L/min, nebulizer pressure=45 psi, sheath gas temperature=375° C., sheath gas flow=12 L/min, fragmentor voltage=100 V, and capillary voltage=3.5 kV. A typical run consisted of 33 minutes, with the following solvent run sequence post injection: 0.1 ml/min 0% buffer B for 5 minutes, 0.4 ml/min linear gradient of buffer B from 0-100% over 15 minutes, 0.5 ml/min of 100% buffer B for 8 minutes, and re-equilibration with 0% buffer B for 5 minutes. All the species were quantified by measuring the area under the curve in comparison to the appropriate unnatural internal standard, and then normalizing to with wet tissue weight (tissue samples) or number of cells (cellular and secreted samples). All the data is represented as mean±s.e.m. of between 4-8 biological replicates depending on the experiment.

### Example 31: Harvesting and Treating Thioglycollate-Elicited Macrophages

Peritoneal macrophages were elicited and harvested from mice according to the standard protocols available from LIPID MAPS (See http://www.lipidmaps.org/protocols/PP0000001400.pdf and http://www.lipidmaps.org/protocols/PP0000001500.pdf). After removal of the RBC lysis buffer, the macrophages were re-suspended in RPMI 1640 with 10% FCS and IX penicillin-streptomycin. Cell counts measured using a Bio-Rad TC10 automated cell counter, and equal numbers of cells (2×10⁶) were plated in 10 cm tissue culture dishes. Macrophages were allowed to adhere to the plates for 2 h at 37° C. and 5% CO₂. The macrophages were then washed three times with sterile PBS to remove any cellular debris, and the treated with KC01 or KC02 (1 μM) or DMSO (control) for 4 h in 4.5 mL of serum free phenol red free RPMI 1640 medium. Macrophages were then stimulated with lipopolysaccharide (5 μg/mL) for 7 hours. Sterile PBS was the vehicle in these studies. Four milliliters of the media was used for measuring secreted metabolites, and 0.5 mL was used for cytokine measurements.

Macrophages were harvested by scraping and washed twice with sterile cold PBS before further use. All samples were either used immediately or flash frozen in liquid N₂, and stored at −80° C. until further use.

### Example 32: Biochemical Characterization of PS Lipase Activity in Mouse Brain

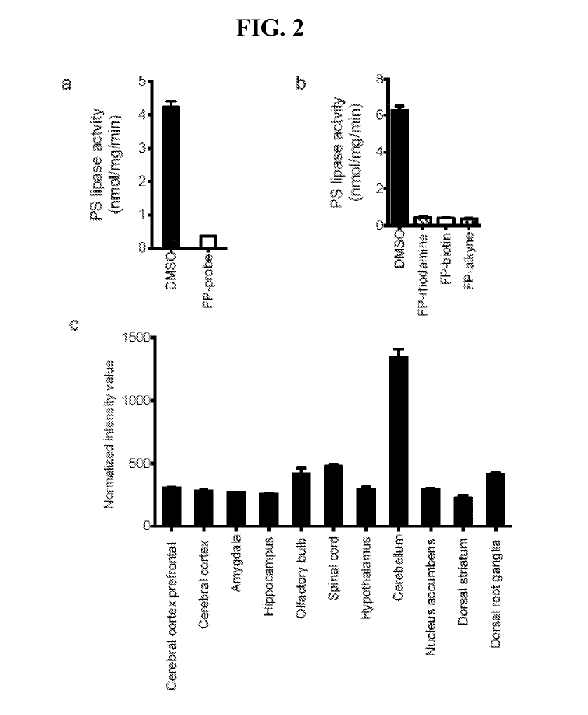

Mouse brain proteomes (1 mg/mL) were treated with DMSO or serine hydrolase-directed activity-based fluorophosphonate-rhodamine (FP-probe; 20 μM, 30 min 37° C.) and then assayed for PS lipase activity with a C18:0/C18:2 PS substrate (100 μM). Reactions were quenched and extracted with 2:1 CHCl₃:CH₃OH, and the organic extracts analyzed by LC-MS as described herein. Data is shown in FIG. 2a. (Data represent mean values±s.e.m. for three biological replicates.) Inhibition of mouse brain membrane fraction PS lipase activity by different FP-probes, assayed as described above is shown in FIG. 2b. (Data represent mean values±s.e.m. for three biological replicates.) BioGPS gene expression profile for mouse Abhd16a in various regions of the brain and nervous system (http://biogps.org/) is shown in FIG. 2c.

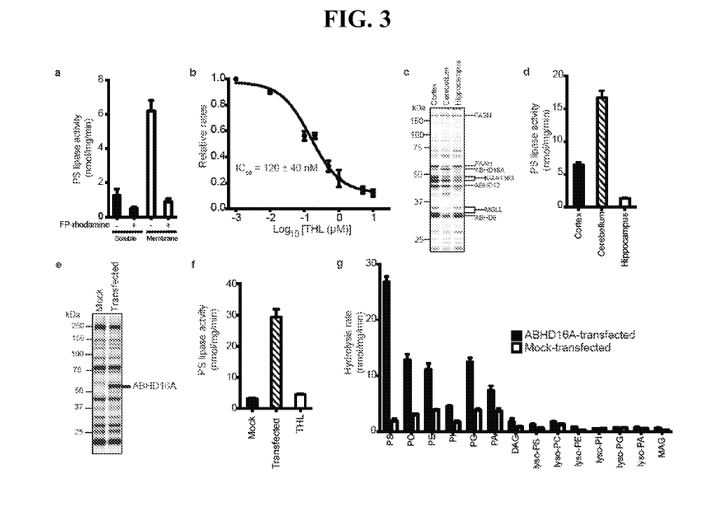

PS lipase activity of mouse brain soluble and membrane proteomes after pre-treatment with DMSO or FP-rhodamine (10 μM, 30 min, 37° C.) is shown in FIG. 3a. (Data represent mean values±s.e.m. for three biological replicates.)

Concentration-dependent inhibition of brain membrane PS lipase activity by tetrahydrolipstatin (THL23; also known as Orlistat) is shown in FIG. 3b. Data represent mean values±s.e.m. for three biological replicates, and the 95% interval for the reported IC₅₀ value is 40-210 nM.

ABPP gel of the membrane proteomes (1 mg protein/mL) of the different mouse brain regions treated with FP-rhodamine (2 μM, 30 min, 37° C.) is shown in FIG. 3c.

Representative brain serine hydrolases are designated.

PS lipase activity of membrane proteomes from mouse brain regions is shown in FIG. 3d. Data represent mean values±s.e.m. for three biological replicates.

ABPP-gel of membrane proteomes from mock- and ABHD16A-transfected HEK293T cells, showing robust expression and activity of the recombinant mouse ABHD16A enzyme, is depicted in FIG. 3e.

PS lipase activity of membrane proteomes of mock- and ABHD16A-transfected HEK293T cells is shown in FIG. 3f. The heightened PS lipase activity of the ABHD16A-transfected proteome was blocked by pre-treatment with THL (20 μM, 30 min, 37° C.) Data represents mean values±s.e.m. for three biological replicates.

Results for in vitro lipid substrate hydrolysis assays for membrane proteomes of mock and murine ABHD16A transfected HEK293T proteomes is shown in FIG. 3g. Data represent mean values±s.e.m. for two biological replicates. See Table 2 for complete details of data sets of lipid substrate hydrolysis assays.

Data Analysis

LC-MS assay results for monitoring conversion of PS to lyso-PS indicated that the mouse brain proteome possesses an sn-2-selective PS lipase activity that generates lyso-PS and free fatty acid (FFA) products. The majority of this activity was membrane-associated (FIG. 3a) and blocked by pre-treatment with FP probes (e.g., FP-rhodamine, FP-biotin). The mouse brain PS lipase activity was blocked by the general serine lipase inhibitor THL with an IC₅₀ value of 120±40 nM. See FIG. 3b.

THL has previously been shown to inhibit several brain serine hydrolases using competitive activity-based protein profiling (ABPP) assays, including ABHD12 and an uncharacterized 60 kDa membrane enzyme tentatively identified as ABHD16A/BAT5, both of which exhibited IC₅₀ values of −100 nM. ABHD12 was previously demonstrated to not exhibit PS lipase activity and therefore attention was turned to ABHD16A. Consistent with ABHD16A potentially exhibiting PS lipase activity, the cerebellum was found to possess substantially higher amounts of this enzyme (ABPP analysis; FIG. 3c) and greater PS lipase activity (FIG. 3d) compared to other brain regions. Most other brain serine hydrolases were not enriched in the cerebellum relative to other brain regions (e.g., cortex, hippocampus; FIG. 3c). Large-scale gene expression studies (http://biogps.org) also support the enriched expression of ABHD16A in mouse cerebellum (FIG. 2c)

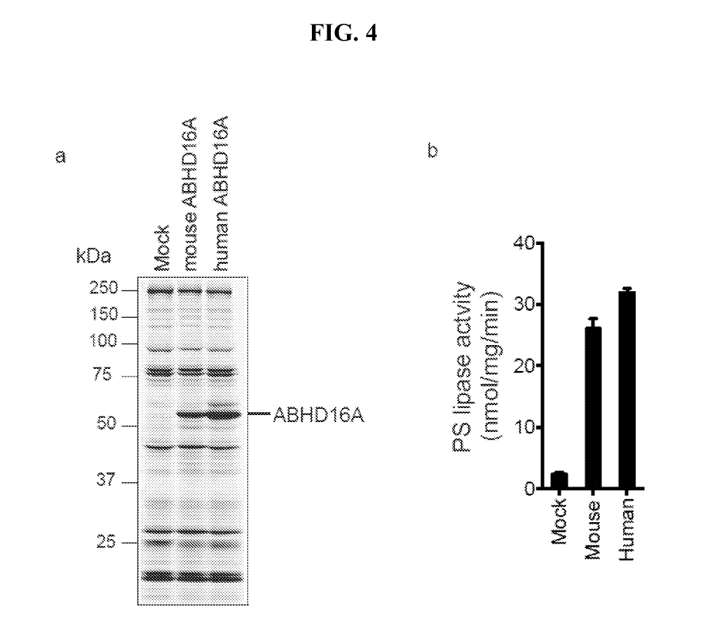

Recombinant expression of mouse and human ABHD16A by transient transfection in HEK293T cells was performed as described herein and/or as follows. Gel-based ABPP experiments were performed by labeling 1 mg/mL of each proteome with FP-rhodamine (2 μM, 30 min, 37° C.). Reactions were then quenched using SDS-PAGE loading buffer, separated by SDS-PAGE, and enzyme activities detected by in-gel fluorescence scanning (fluorescent gel shown in gray scale). Gel-based ABPP experiments were performed in triplicate with consistent results. Gel-based ABPP confirmed robust expression of ABHD16A proteins in transfected cells compared to mock-transfected counterparts (see FIG. 3e and FIG. 4).

PS-lipase activity of mock-, mouse ABHD16A-, and human ABHD16A-transfected HEK293T membrane proteomes was measured using a C18:0/C18:2 PS substrate (100 μM) (FIG. 3f and FIG. 4b). Assay was performed as described for FIG. 2. Data represent mean values±s.e.m. for three biological replicates. Mouse and human ABHD16A-transfected cell membrane proteomes displayed nearly ten-fold higher PS lipase activity compared to mock-transfected cell membranes, and this heightened activity was blocked by pretreatment with THL (20 μM, 30 min). See FIG. 3f and FIG. 4. Mouse ABHD16A-transfected cell membrane preparations was also tested for activity with various mono- and diacyl lipid substrates, which revealed that ABHD16A shows highest specific activity with PS (Kₘ=40±5 μM and Vₘₐₓ=35±3 nmol/mg/min for a C18:0/C18:2 PS substrate) and lower activity with other diacylated phospholipids, but negligible activity with lysophospholipids and neutral lipids (FIG. 3g).

Taken together, these biochemical data indicate that ABHD16A is a major PS lipase of mouse brain tissue.

### Example 33: Screening of α-Alkylidene-β-Lactones Against Human ABHD16A by Competitive ABPP

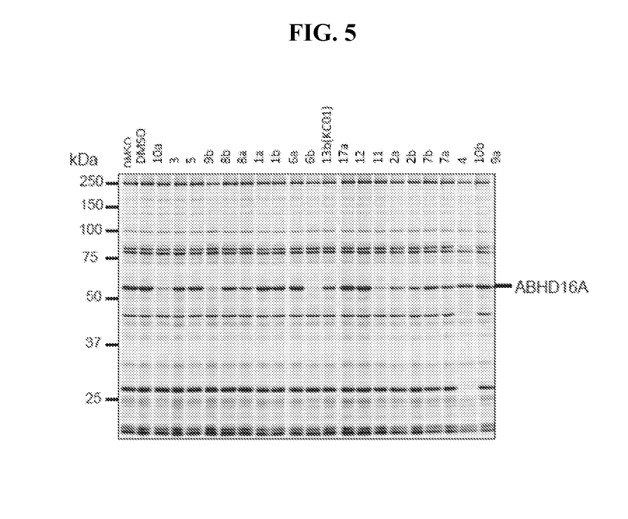

Recombinant human ABHD16A was assayed against a focused library of α-methylene-β-lactones or reduced versions of these compounds. Human ABHD16A-transfected HEK293T cell proteome (1 mg/mL) was pre-treated with α-alkylidene-β-lactones (10 μM, 30 min 37° C.) and then incubated with FP-rhodamine (2 μM, 30 min, 37° C.), and the samples were analyzed by gel-based ABPP (as described herein). Inhibitory activity was detected by loss of FP-rhodamine labeling of ABHD16A, and the selectivity of inhibitors was assessed by loss of other serine hydrolase activity signals detected by gel-based ABPP. Gel-based ABPP experiments were performed in duplicate with consistent results. Data are shown in FIG. 5.

### Example 34: Identification of an ABHD16A Inhibitor and a Paired Inactive Control Probe

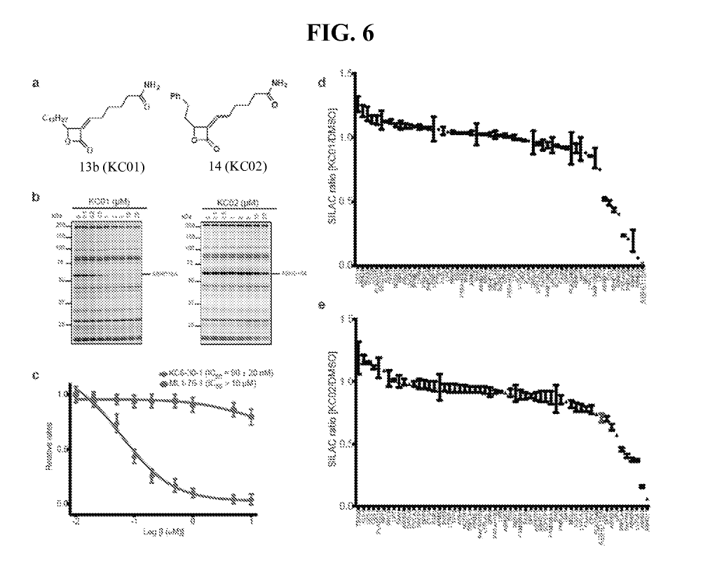

Competitive ABPP gels showing the concentration-dependent inhibition of ABHD16A by KC01 (compound 13b), but not KC02 (compound 14), in ABHD16A-transfected HEK293T cell proteomes are shown in FIG. 6b. Proteomes were treated with inhibitors for 30 min at 37° C., followed by FP-rhodamine (2 μM, 30 min, 37° C.).

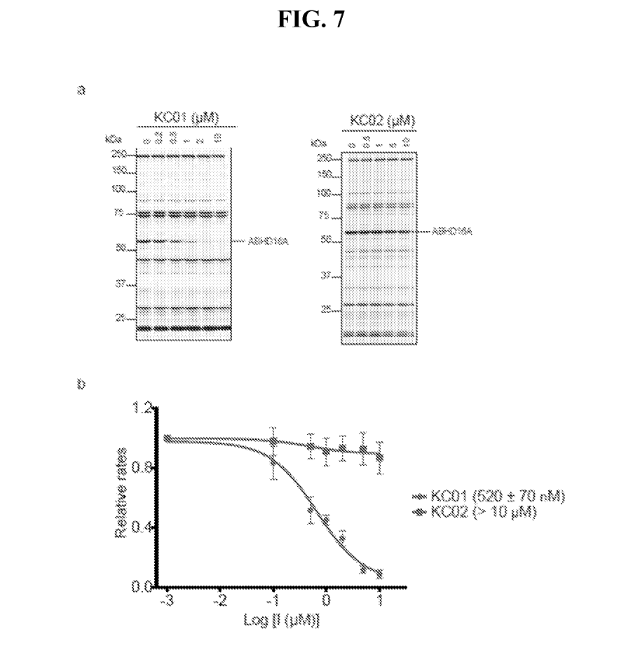

As shown in FIG. 7a, specific concentrations of KC01 and KC02 were incubated with mouse ABHD16A-transfected HEK293T cell proteomes (1 mg/mL) for 30 min at 37° C., and then samples were treated with FP-rhodamine (2 μM, 30 min, 37° C.), and analyzed by gel-based ABPP. From the data, an IC₅₀ value of ˜0.5 μM was calculated for inhibition of mouse ABHD16A by KC01. KC02 did not inhibit mouse ABHD16A across the tested concentration range (IC₅₀>10 μM). Gel-based ABPP experiments were performed in triplicate with consistent results. As shown in FIG. 7b, indicated concentrations of KC01 and KC02 were incubated with mouse ABHD16A-transfected HEK293T cell proteomes (30 min, 37° C.), and thereafter assayed using a C18:0/C18:2 PS substrate (100 μM). Assay was performed as described in Example 32. The IC₅₀ value obtained from this assay was 520±70 nM for KC01. KC02 did not inhibit PS-lipase activity of mouse ABHD16A (IC₅₀>10 μM). Data represent mean values±s.e.m. for three biological replicates.

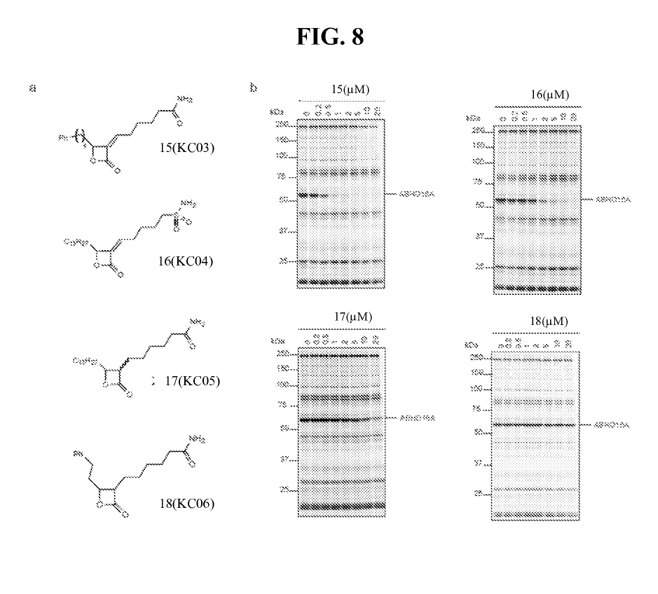

As shown in FIG. 8, varying concentrations of KC01 analogs were incubated against human ABHD16A-transfected HEK293T proteomes (1 mg/mL) for 30 min at 37° C., and then samples were treated with FP-rhodamine (2 μM, 30 min, 37° C.), and analyzed by gel-based ABPP. From the data, the following IC₅₀ values were estimated: 23 (KC03) (0.2-0.5 μM), 24 (KC04) (0.5-1 μM), 25 (KC05) (5 μM), and 26 (KC06) (>20 μM) for ABHD16A. Gel-based ABPP experiments were performed in duplicate with consistent results.

Data Analysis

These further ABPP experiments designated KC01 (compound 13b) (FIG. 6a) as the most potent and selective of the inhibitors for ABHD16A (FIG. 6b and FIG. 7). Generation of structural analogues of KC01 (FIG. 8), led to the discovery of an inactive “control” probe KC02 (compound 14) (FIG. 6a). The synthesis of KC02 consistently yielded a 4:1 Z/E isomers, which proved difficult to chromatographically separate. Therefore, KC02 was used as a 4:1 Z/E mixture. Competitive gel-based ABPP was used to measure IC₅₀ values of ˜0.2-0.5 μM and >10 μM for the inhibition of ABHD16A (human and mouse) by KC01 and KC02, respectively (FIG. 6b and FIG. 7).

Similar IC₅₀ values were calculated using a PS substrate assay (KC01 IC₅₀=90±20 nM for human ABH16A; IC₅₀=520±70 nM for mouse ABHD16A; KC02 IC₅₀>10 μM for both human and mouse ABHD16A; FIG. 6c and FIG. 7).

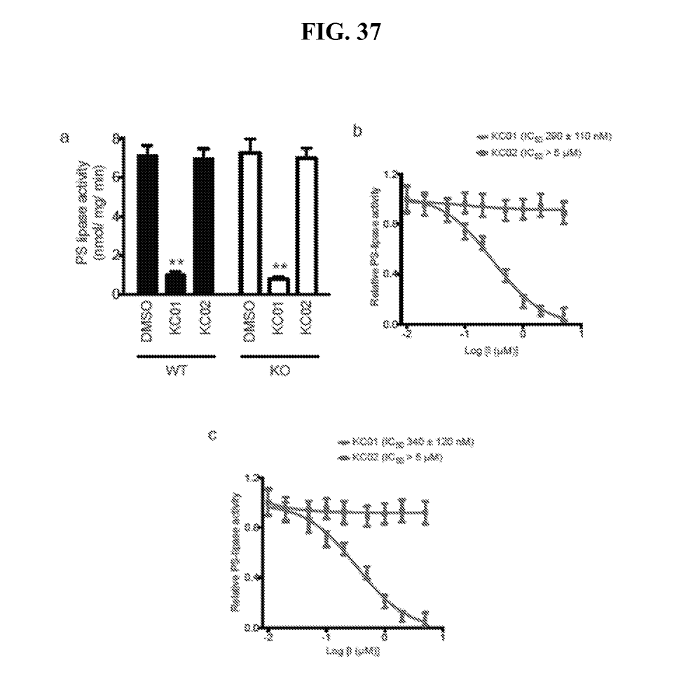

KC01, but not KC02, inhibited the PS lipase activity of brain membrane lysates from two-month old ABHD12^{+/+} and ABHD12^{-/-} mice (FIG. 37).

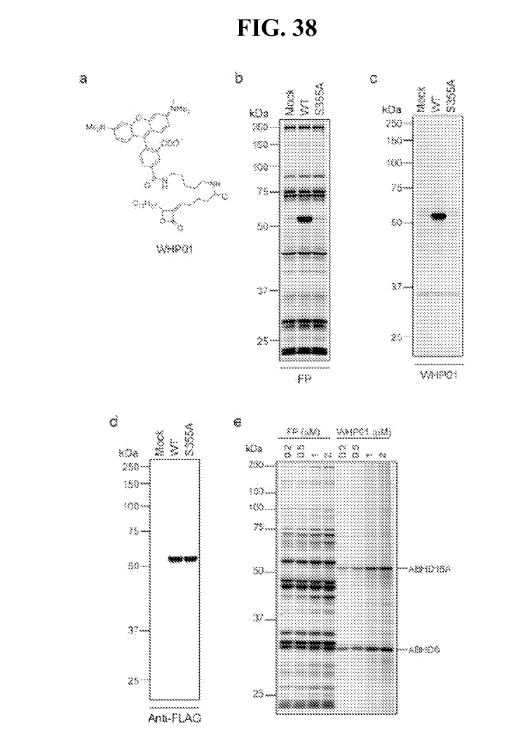

Consistent with the predicted mechanism of covalent inhibition of ABHD16A by KC01, a fluorescent analog of KC01, termed WHP01 (compound 19), labeled WT-ABHD16A but not the catalytic serine S355A mutant of this enzyme in transfected HEK293T cell lysates (FIG. 38). WHP01 also served as a sensitive probe for detecting ABHD16A activity in mouse brain membrane lysates (FIG. 38).

### Example 35: In Situ Inhibition of ABHD16A Using an ABHD16A Inhibitor

Confirmation of strong ABHD16A activity in the membrane fraction of three human cancer cell lines—COLO205 (colon cancer), K562 (leukemia) and MCF7 (breast cancer)—by the MS method ABPP-MudPIT (see Jessani et al. Nat. Methods (2005) 2, 691-697) (data not shown) was first obtained.

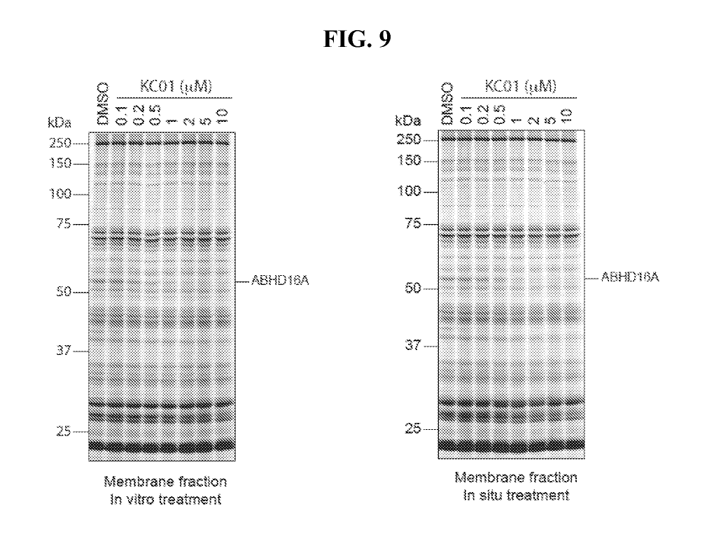

The K562 cell line was treated with varying concentrations of KC01 for 4 h and the cell membrane fractions analyzed by gel-based ABPP. More specifically, the membrane proteome of K562 cells (left gel, FIG. 9) or cultured K562 cells (right gel, FIG. 9) were treated with the indicated concentrations of KC01 (in vitro: 1 mg/mL proteome; 30 min treatment, 37° C.; in situ: 2×10⁶ cells; 4 h treatment, 37° C.), followed by FP-rhodamine (added in vitro to cell proteomes at 2 μM for 30 min, 37° C.) and analyzed by gel-based ABPP. Estimated in vitro and in situ IC₅₀ values for KC01 from the gel-based ABPP experiments were 0.2-0.5 μM. Gel-based ABPP experiments were performed in triplicate with consistent results.

The results confirmed in situ inhibition of ABHD16A with an IC₅₀ value of −0.3 μM (FIG. 9). Very few off-targets were detected for KC01 in this gel-based ABPP experiment.

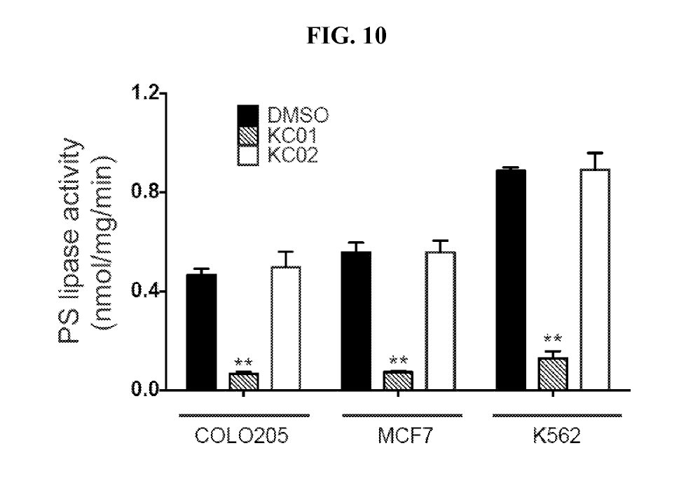

To more thoroughly assess the selectivity of KC01, quantitative MS experiments were performed using the ABPP-SILAC method. For this study, the COLO205 cell line was investigated, which expresses a rich diversity of serine hydrolases. Cells isotopically labeled with light and heavy amino acids were treated with inhibitors (KC01 or KC02, 1 μM) or DMSO for 4 h at 37° C., lysed, and serine hydrolase activities labeled and enriched with a biotinylated FP probe (FP-biotin33) and analyzed by LC-MS using an LTQ-Orbitrap Velos instrument. Among the 60+ serine hydrolase activities quantified in these experiments, ABHD16A was the most potently inhibited enzyme in KC01-treated cells (98% inhibition; light:heavy ratio of 0.02; FIG. 6d), followed by ABHD2 (94%) and a handful of additional off-targets that were partially inhibited between 50-80% (FIG. 6d). Most of these enzymes were also inhibited by the control probe KC02, with the exceptions of two partial off-targets of KC01-ABHD3 and ABHD13 (FIG. 6e). KC02 also inhibited ABHD11 (94%) and LYPLA1 (63%), but did not substantially inhibit ABHD16A (<30%; FIG. 6e). Finally, in situ treatment with KC01 (1 μM, 4 h) but not KC02 (1 μM, 4 h) blocked the PS lipase activity of membrane fractions from COLO205, K562, and MCF7 cell lines (FIG. 10). This latter study was performed in the following manner: the COLO205 (colon cancer), MCF7 (breast cancer) and K562 (leukemia) cancer cell lines were treated in situ with inhibitors (KC01 or KC02, 1 μM) or DMSO for 4 h. Cells were then harvested, lysed, and their membrane proteomes tested for PS-lipase activity using a C18:0/C18:2 PS substrate as described in Example 32. For each cancer cell line, the PS-lipase activity was substantially decreased (>90%) by KC01, but KC02 compared to DMSO controls. Data represent mean values±s.e.m for three biological replicates. Student's t-test: ** p<0.0005 for KC01-treated versus DMSO-treated cancer cells.

### Example 36: Disruption of ABHD16A Impairs PS Metabolism in Human Cells

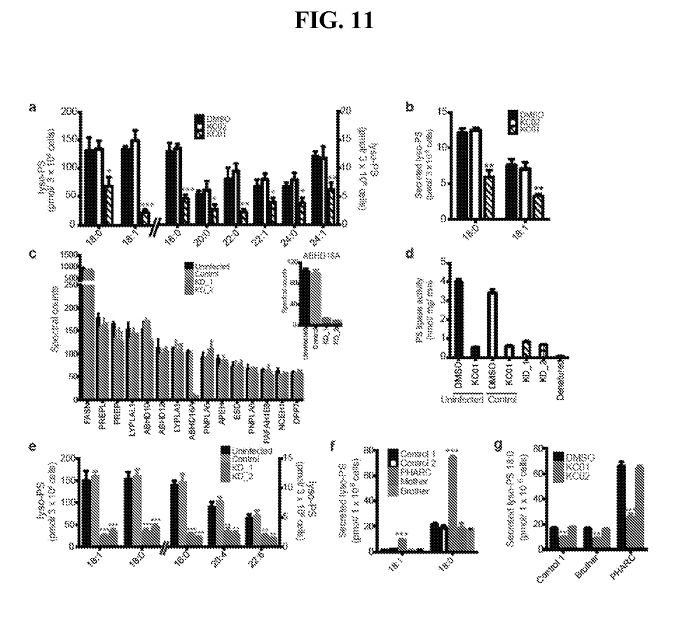

As shown in FIGS. 11a and 11b, cellular (a) and secreted (b) concentrations of lyso-PS from COLO205 colon cancer cells were treated in situ with inhibitors (KC01 or KC02, 1 M) or DMSO for 4 h. Data represent mean values±s.e.m; N=8 per group, Student's t-test: * p<0.05, ** p<0.005, *** p<0.0001 for KC01-treated versus DMSO-treated cells.

Spectral count values of select serine hydrolase activities of the membrane proteomes of mock, control-shRNA, KD_1-shRNA and KD_2-shRNA K562 leukemia cell line models as measured by ABPP-MudPIT are shown in FIG. 11c. Inset of FIG. 11c shows a blow-up of the ABHD16A spectral counts. Data represent mean spectral count values±s.e.m for three biological replicates for the 15 most abundant serine hydrolase activities.

Shown in FIG. 1id are PS lipase activities of membrane proteomes from the indicated K562 cell lines. The uninfected and control cell line proteomes were pre-treated with DMSO or KC01 (1 μM, 30 min, 37° C.) to establish PS lipase activities for baseline and ABHD16A-inhibited samples. The uninfected proteome was also heat-denatured prior to analysis as an additional control. Data represent mean values±s.e.m for three biological replicates.

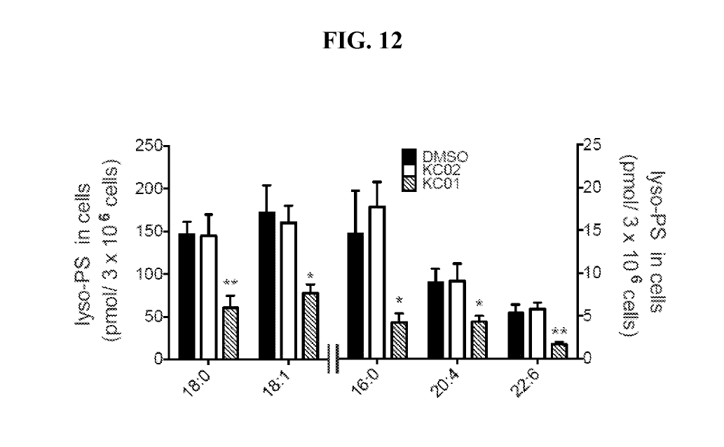

As shown in FIG. 12, K562 cells were treated with inhibitors (KC01 or KC02, 1 M) or DMSO for 4 h, and changes in cellular lyso-PS lipids were measured by targeted multiple reaction monitoring (MRM) methods as detailed herein. Data represent mean values±s.e.m.; N=8 per group. Student's t-test: * p<0.05, ** p<0.005 for KC01-treated versus DMSO-treated cells.

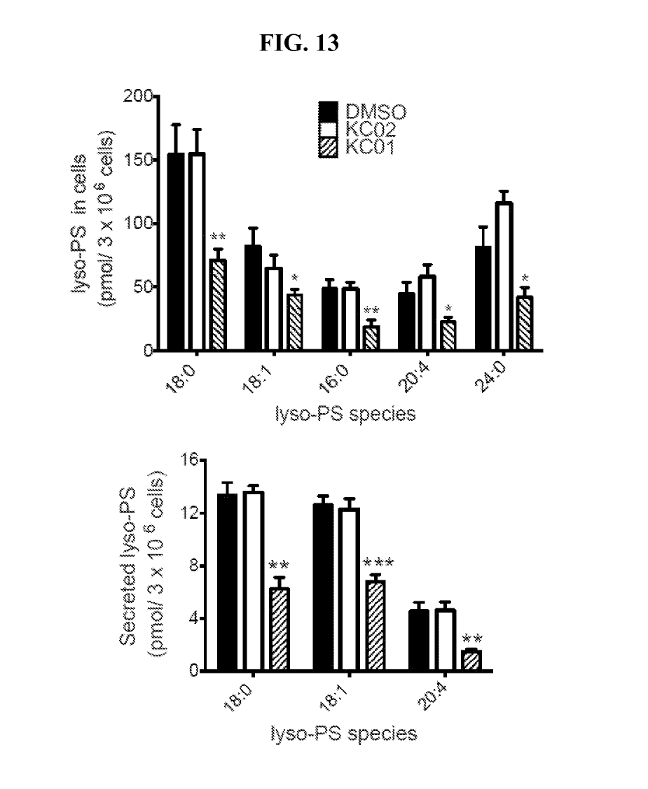

As shown in FIG. 13, MCF7 cells were treated with inhibitors (KC01 or KC02, 1 M) or DMSO for 4 h, and changes in cellular (upper graph) and secreted (lower graph) lyso-PS lipids were measured by targeted multiple reaction monitoring (MRM) methods as detailed herein. Data represent mean values±s.e.m.; N=8 per group. Student's t-test: * p<0.05, ** p<0.005, *** p<0.0001 for KC01-treated versus DMSO-treated cells.

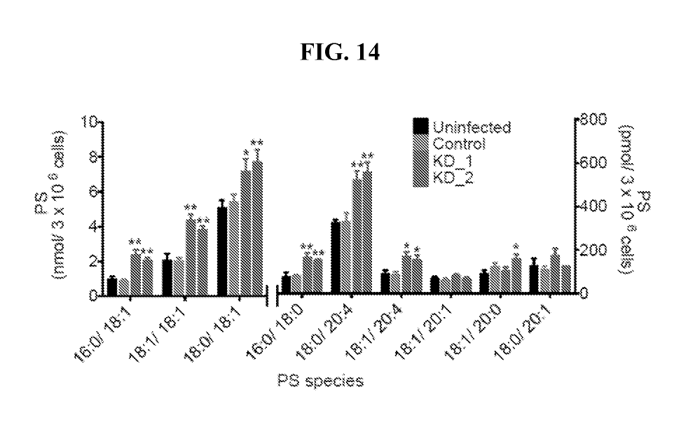

PS content of K562 cells expressing different shRNA knockdown constructs is shown in FIG. 14. PS lipids were measured by targeted multiple reaction monitoring (MRM) methods as detailed herein. Data represent mean values±s.e.m.; N=8 per group. Student's t-test: * p<0.05, ** p<0.005 for KD_1 or KD_2 versus uninfected or control cells.

Data Analysis

LC-MS-based lipid profiling was performed of COLO205 cells treated with inhibitors (KC01 or KC02, 1 μM) or DMSO for 4 h and found that KC01-treated cells, but not KC02-treated cells, showed significant reductions in all detected cellular lyso-PSs compared to DMSO-treated control cells (FIG. 1a). No changes were detected in other lipids, including PSs, PCs, PEs, lyso-PCs, lyso-PEs, or monoacylglycerols (MAGs), in KC01-treated cells (data not shown). Secreted lyso-PSs (18:1 and 18:0) were also decreased in COLO205 cells treated with KC01 compared to KC02- or DMSO-treated cells (4 h treatments in serum-free media; FIG. 11b), while other secreted lipids (lyso-PCs, lyso-PEs, MAGs) were unchanged across these treatment groups (data not shown). Similar changes in cellular and secreted lyso-PSs were observed in K562 (FIG. 12) and MCF7 (FIG. 13) cells treated with KC01, but not KC02.

As a complement to the pharmacological studies, ABHD16A expression in K562 cells was also knocked down using RNA-interference technology. Two stable shRNA knockdown lines targeting ABHD16A (KD_1 and KD_2) and a control line expressing a scrambled shRNA probe were generated and confirmed near-complete (˜90%) and selective reductions in ABHD16A in the KD_1 and KD_2 lines compared to uninfected or control lines by ABPP-MudPIT analysis (FIG. 11c). None of the other 35+ serine hydrolase activities measured in these experiments were affected in the KD_1 and KD_2 lines.

ABHD16A knockdown greatly reduced the PS lipase activity of K562 cells to levels observed in uninfected cells treated with KC01 (FIG. 11d). Lipid profiling revealed that cellular lyso-PSs were substantially decreased in the KD_1 and KD_2 lines compared to uninfected or control lines (FIG. 11e). Notably, several PSs were conversely elevated in the KD_1 and KD_2 lines (FIG. 14), while other major lipids (PCs, PEs, lyso-PCs, lyso-PEs, and MAGs) were unchanged (data not shown). It was not possible to analyze secreted lipids in the shRNA lines, as they showed poor viability when cultured in serum-free media.

The pharmacological and shRNA data, taken together, indicate that ABHD16A is a principal PS lipase in human cancer cells, where acute inactivation of the enzyme produced primarily a decrease in lyso-PSs, while prolonged reductions in enzyme expression caused both decreases in lyso-PSs and elevations in PSs.

### Example 37: Interplay Between ABHD16A and ABHD12 Control Lyso-PS Tone in Human Cells

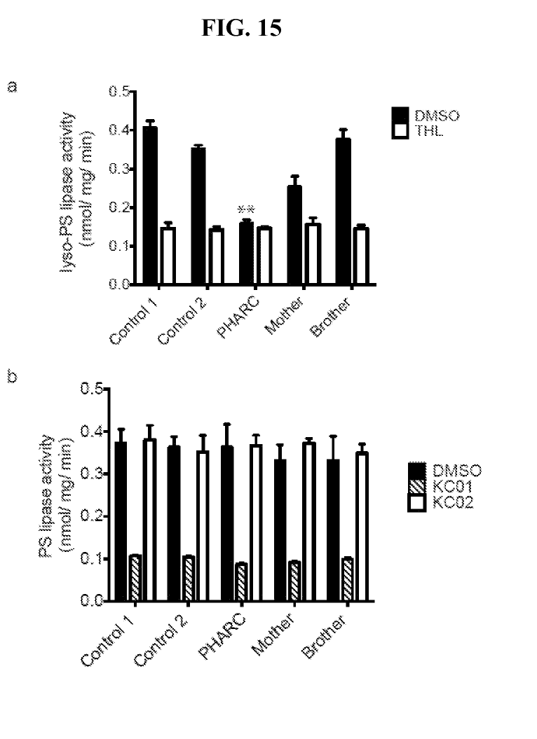

Lyso-PS lipase activity of membrane proteomes from indicated LCLs is shown in FIG. 15a. Proteomes were pre-treated with DMSO or THL (10 μM, 30 min, 37° C.) and then assayed for lyso-PS lipase activity using a C18:1 lyso-PS substrate (100 μM) as described in Blankman et al. (Proc. Natl. Acad. Sci. USA (2013) 110, 1500-1505).

PS lipase activity of membrane proteomes from indicated LCLs is shown in FIG. 15b. Proteomes were pre-treated with inhibitors (KC01 or KC02, 1 μM) or DMSO for 30 min at 37° C. and then assayed for PS lipase activity using a C18:0/C18:2 PS substrate (100 μM) as described in Example 32. Note that KC01, but not KC02 substantially reduced the PS lipase activity of each LCL. Data represent mean values±s. e. m for three biological replicates. For part (a), Student's t-test: ** p<0.005 for PHARC LCL versus Control 1 or Control 2 LCLs.

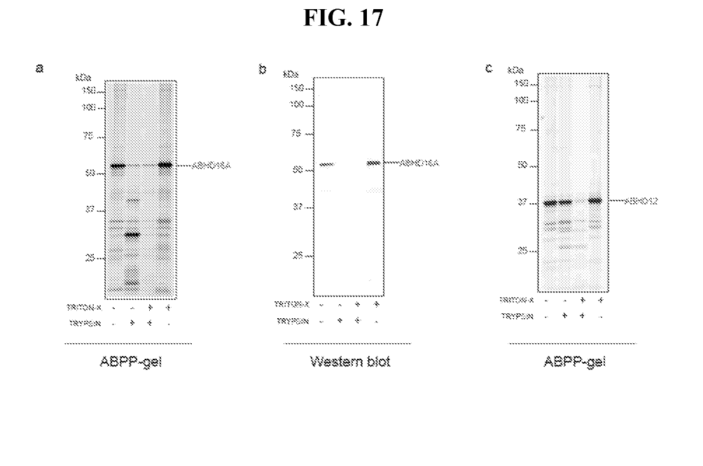

Protease protection assays for ABHD16A and ABHD12 were performed in the following manner. A C-terminal FLAG-tagged human ABHD16A construct was transfected into HEK293T cells, and the membrane proteome (1 mg/mL) of these cells was labeled with FP-rhodamine (2 μM 30 min, 37° C.) and treated with trypsin in the presence or absence of the detergent Triton-X. ABHD16A was susceptible to trypsin digestion with or without detergent as measured by gel-based ABPP (FIG. 17a) or anti-FLAG blotting (FIG. 17b), indicative of a cytosolically-oriented membrane protein. In contrast, ABHD12 only showed sensitivity to trypsin digestion in the presence of detergent (FIG. 17c), indicative of a lumenally-oriented membrane protein, as described in Blankman et al. (Chem. Biol. (2007) 14, 1347-1356). Gel-based ABPP and western blotting experiments were performed in duplicate with consistent results.

Data Analysis

In order to determine whether ABHD16A inhibition altered lyso-PS metabolism in a lymphoblast cell line (LCL) derived from a PHARC subject with compound heterozygotic mutations in the ABHD12 gene 18, five LCLs were investigated, including lines derived from the PHARC subject, their sibling, who possessed a wild-type ABHD12 gene, their mother, who was heterozygotic for ABHD12 gene mutation, and two control subjects with wild-type ABHD12 status. Selective absence of ABHD12 in the PHARC subject-derived LCL was confirmed by gel-based ABPP, as shown previously, (see Chen et al. Human mutation (2013) 34, 1672-1678), and the membrane lysate from this ABHD12-null LCL also exhibited substantial reductions in lyso-PS lipase activity (FIG. 15). The LCL lysate from the ABHD12-heterozygotic mother showed a 50% reduction in lyso-PS lipase activity compared to wild-type LCL controls (FIG. 15). The PS lipase activity was comparable across all of the LCL samples and was inhibited by KC01, but not KC02 (FIG. 15). These results indicated that ABHD12 and ABHD16A are major lyso-PS and PS lipases, respectively, in the human LCLs.

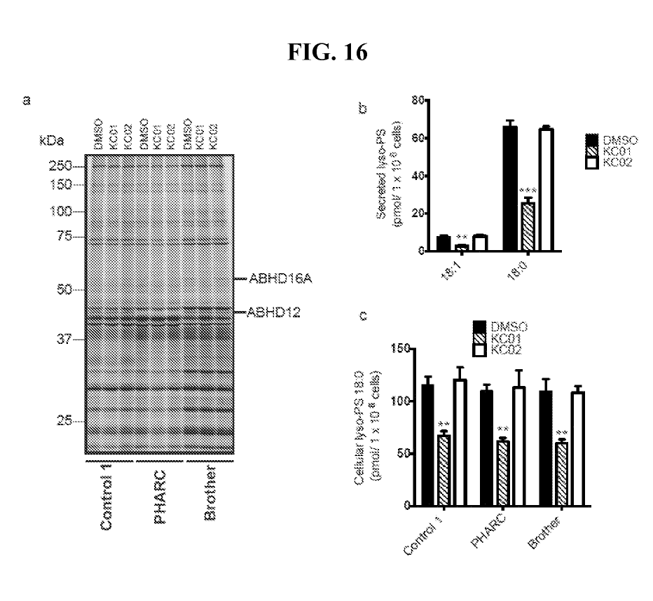

Targeted LC-MS analysis revealed a dramatic elevation in secreted lyso-PSs in the ABHD12-null LCL compared to control LCLs (FIG. 11f and FIG. 16). Other secreted and cellular (lyso)phospholipids were unchanged in the ABHD12-null LCL (data not shown). Treatment with KC01, but not KC02 (1 μM, 4 h) significantly lowered secreted lyso-PSs of the ABHD12-null LCL, such that they nearly matched the concentrations of secreted lyso-PSs of control LCLs, which were also reduced by KC01 treatment (FIG. 11g). All LCLs also showed decreased cellular lyso-PSs following treatment with KC01, but not KC02 (FIG. 16).

Inhibition of ABHD16A in LCLs by KC01 was confirmed, but not KC02 by gel-based ABPP (FIG. 16).

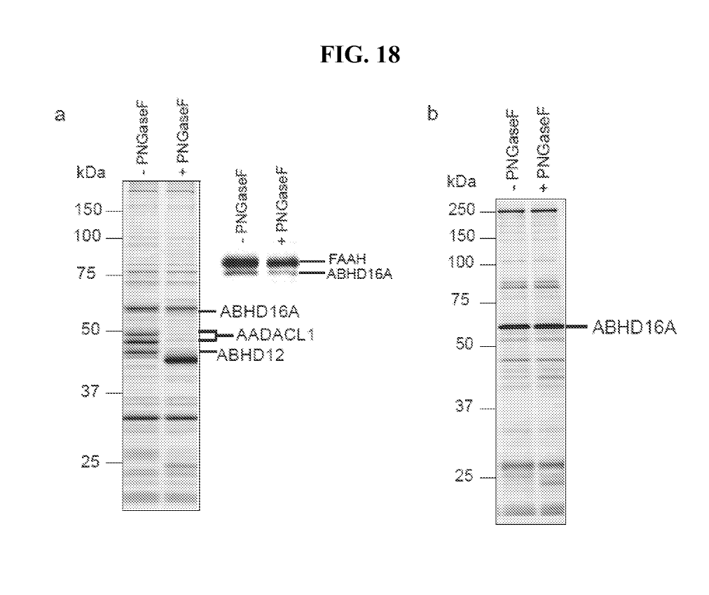

The data indicate that ABHD12 and ABHD16A play complementary roles in the regulation of lyso-PSs in human LCLs, with ABHD16A contributing to the production of both cellular and secreted lyso-PSs and ABHD12 preferentially controlling degradation of secreted lyso-PSs. It has been previously shown that ABHD12 is an extracellularly/lumenally-oriented membrane protein, a localization that is consistent with the enzyme's role in regulating secreted lyso-PSs. The membrane orientation of ABHD16A was examined using a protease protection assay (see Hua et al. J. Biol. Chem. (1995) 270, 29422-29427), which revealed that recombinant ABHD16A expressed in HEK293T cells was susceptible to trypsin degradation in the absence of a membrane-solubilizing detergent Triton X-100 (FIG. 17). This property is consistent with a cytoplasmic membrane orientation for ABHD16A and contrasted with the behavior of recombinant ABHD12, which showed detergent-dependent sensitivity to trypsin degradation (FIG. 17). Also consistent with the respective cytoplasmic and extracellular/lumenal orientations of ABHD16A and ABHD12, only the latter enzyme shows evidence of PNGaseF-sensitive glycosylation (FIG. 18). See Blankman et al. Chem. Biol. (2007) 14, 1347-1356. The localization of ABHD16A on the cytoplasmic face of cellular membranes is consistent with its role as a PS lipase, since PS is largely confined to the inner leaflet of the lipid bilayer in healthy cells36. Although the mechanism(s) that regulate the cellular release of lyso-PS remain unknown, this data argue that, once generated by ABHD16A and secreted from cells, lyso-PS becomes accessible to hydrolytic degradation by ABHD12.

### Example 38: The ABHD16A/ABHD12-Lyso-PS Pathway Regulates Macrophage Inflammatory Responses

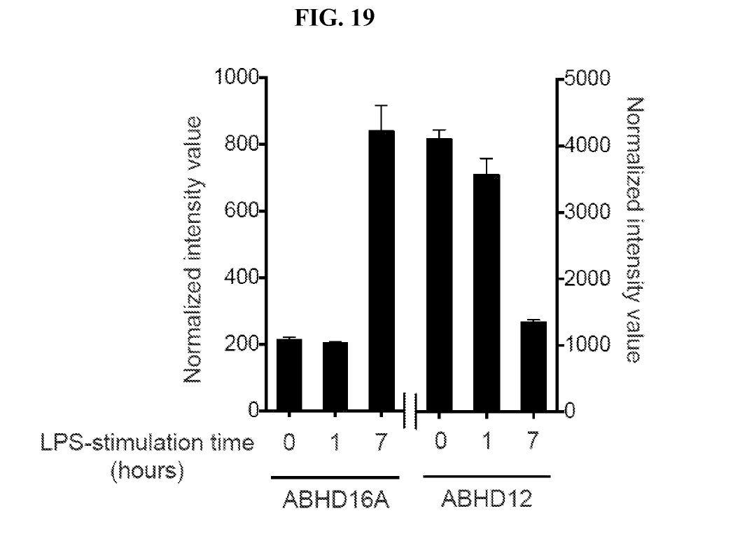

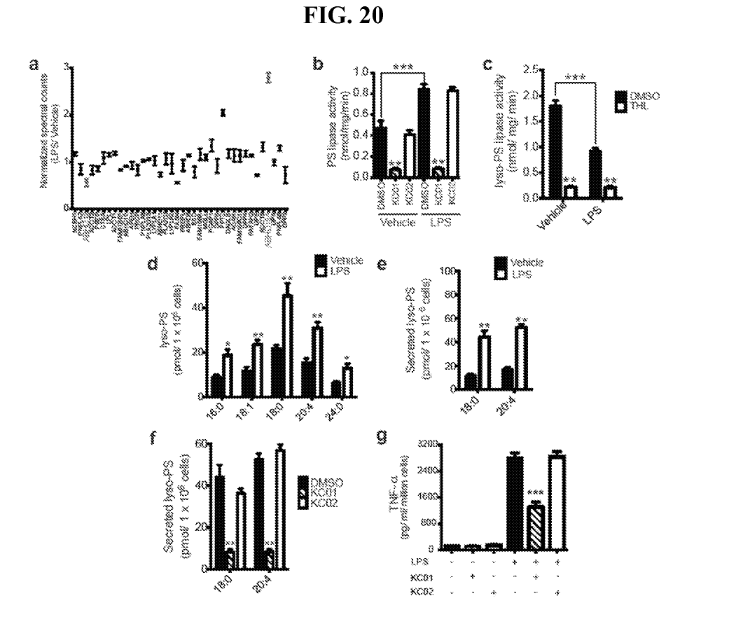

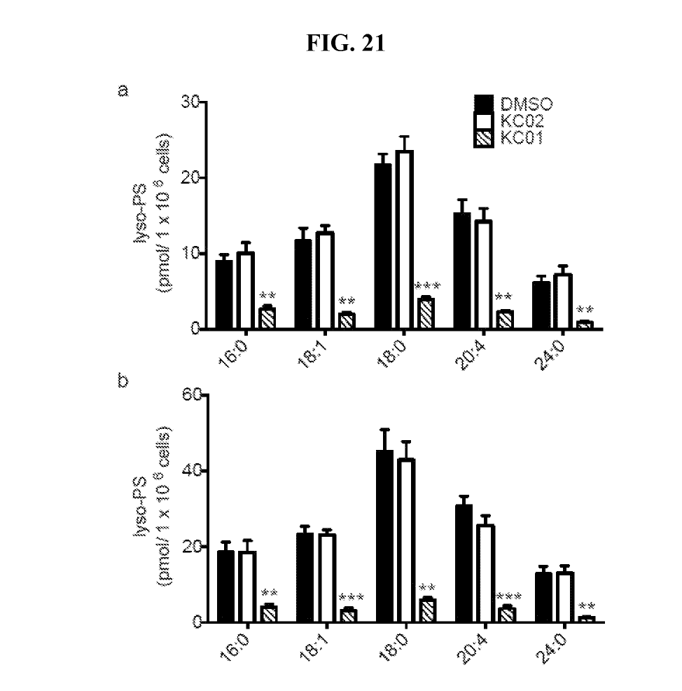

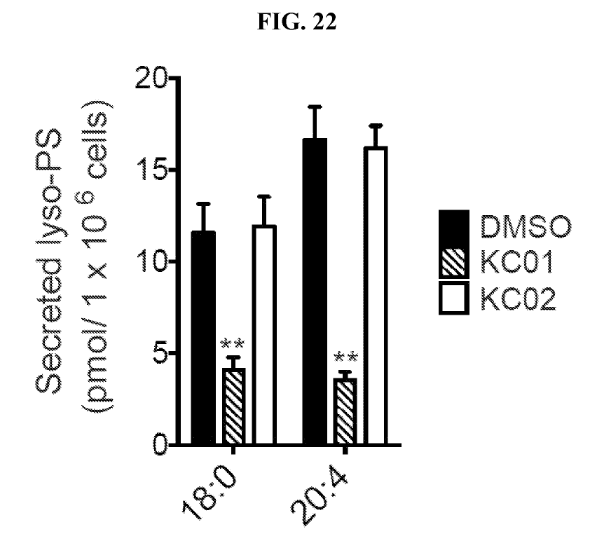

The heightened neuroinflammatory state of ABHD12^{-/-} mice, combined with the immunoregulatory functions of lyso-PS, led to the exploration of the functions of ABHD16A and ABHD12 in immune cells. A survey of public mRNA expression profiles (http://biogps.org/) revealed that ABHD16A and ABHD12 are differentially regulated by lipopolysaccharide (LPS) treatment of mouse peritoneal macrophages (FIG. 19). These findings were verified and extended at the proteomic level by performing a comparative ABPP analysis of thioglycollate-elicited mouse peritoneal macrophages treated with or without LPS for 7 h. Strikingly, among the 40+ serine hydrolases quantified in this study, ABHD16A showed the greatest increase (˜three-fold) in activity, while ABHD12 was one of a handful of serine hydrolases that showed reductions (˜two-fold) in activity (FIG. 20a). A corresponding elevation in PS lipase activity (FIG. 20b) and reduction in lyso-PS lipase activity (FIG. 20c) in LPS-stimulated macrophages was measured, and the former activity was blocked by KC01, but not KC02 (FIG. 20b). The complementary shift in activities for ABHD16A and ABHD12 in LPS-stimulated macrophages resulted in a significant increase in both cellular (FIG. 20d) and secreted (FIG. 20e) lyso-PSs. Other cellular and/or secreted (lyso)phospholipids were unchanged by LPS treatment (data not shown). Both basal and LPS-induced increases in cellular (FIG. 21) and secreted lyso-PSs (FIG. 20f and FIG. 22) were reduced by pre-treatment with KC01 (1 μM, 4 h), but not KC02 (1 μM, 4 h).

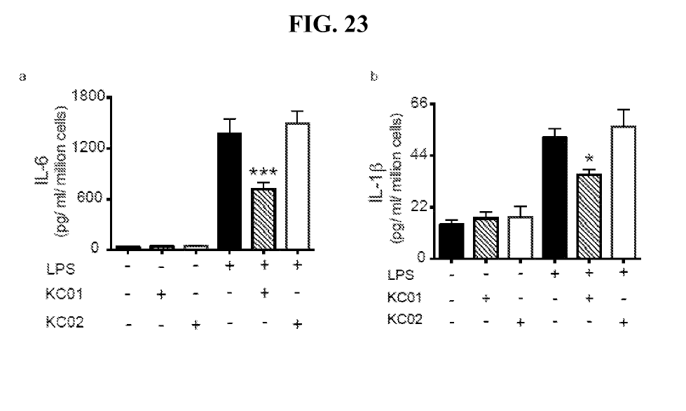

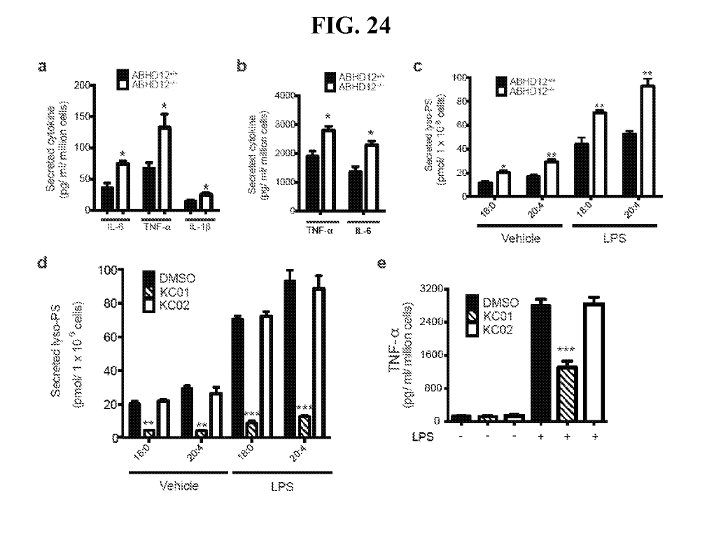

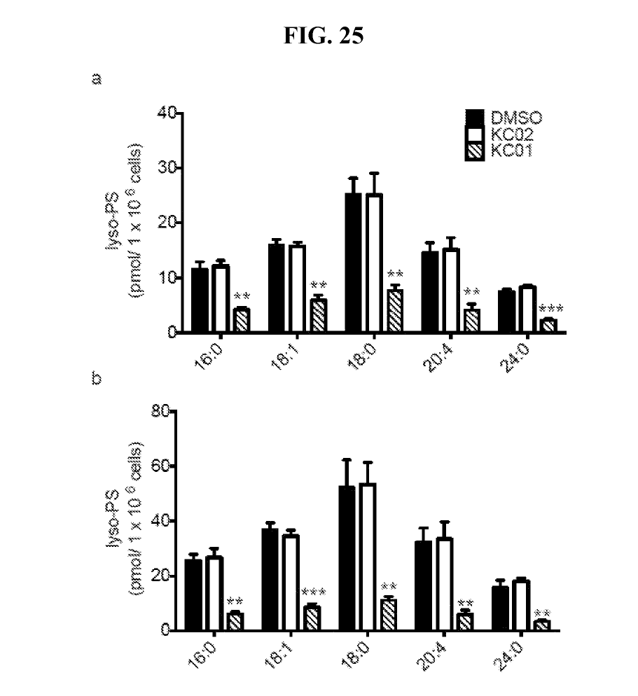

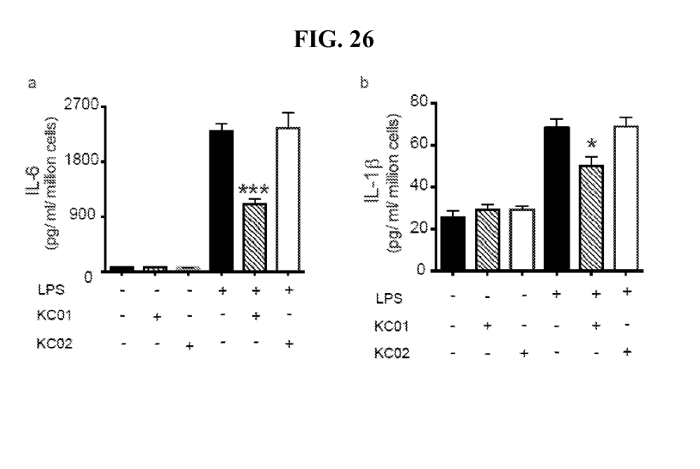

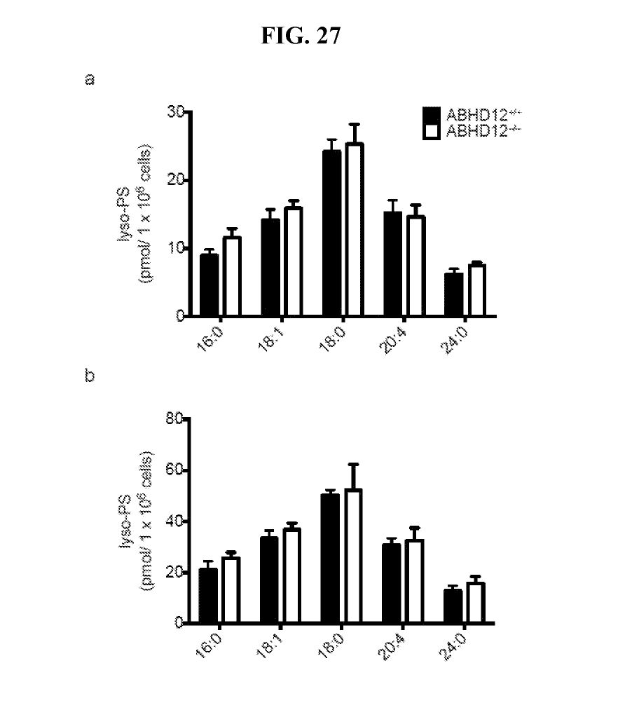

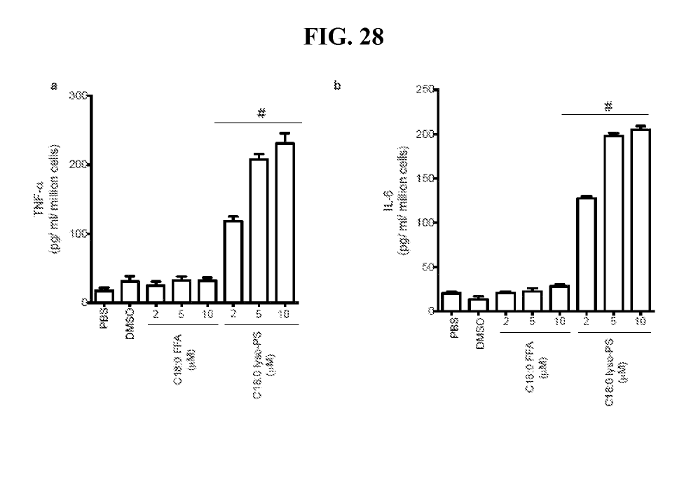

LPS, through activation of innate immune receptors, promotes the release of pro-inflammatory cytokines from macrophages. This LPS-induced cytokine release was significantly blunted by pre-treatment of macrophages with KC01, but not KC02 (FIG. 20g and FIG. 23). Conversely, ABHD12−/− macrophages produced greater amounts of cytokines, both basally (FIG. 24a) and in response to LPS stimulation (FIG. 24b). These cytokine changes correlated with heightened secretion of lyso-PSs from ABHD12-macrophages (FIG. 24c). The LPS-induced increases in lyso-PSs (FIG. 24d and FIG. 25) and cytokine (FIG. 24e and FIG. 26) secretion were both blocked by pre-treatment of ABHD12^{-/-} macrophages with KC01 (1 μM, 4 h), but not KC02 (1 μM, 4 h). As was observed in ABHD12-null LCL cells, ABHD12^{-/-} macrophages did not show alterations in cellular lyso-PS content (FIG. 27). Finally, C18:0 lyso-PS, which represented one of the most abundant lyso-PS species secreted by macrophages, promoted cytokine release when added exogenously to macrophages (FIG. 28).

These data, taken together, indicate that the ABHD16A/ABHD12-lyso-PS pathway is dynamically regulated by immunological stimuli in macrophages and supports pro-inflammatory cytokine release from these cells.

### Example 39: ABHD16A^{-/-} Mice Confirm that ABHD16A is a Principal PS Lipase In Vivo

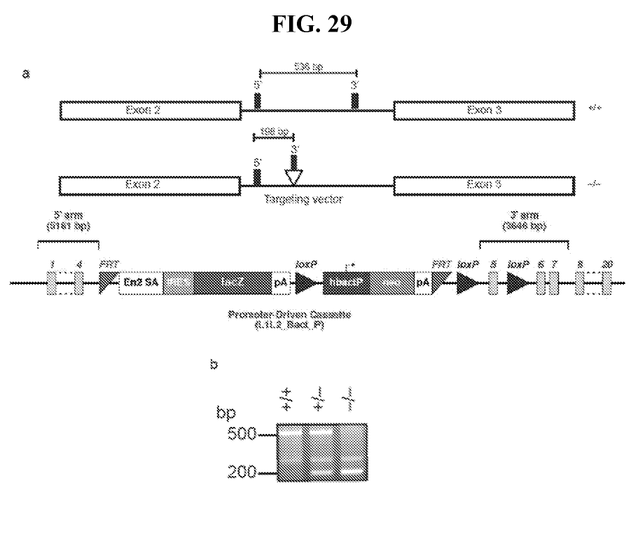

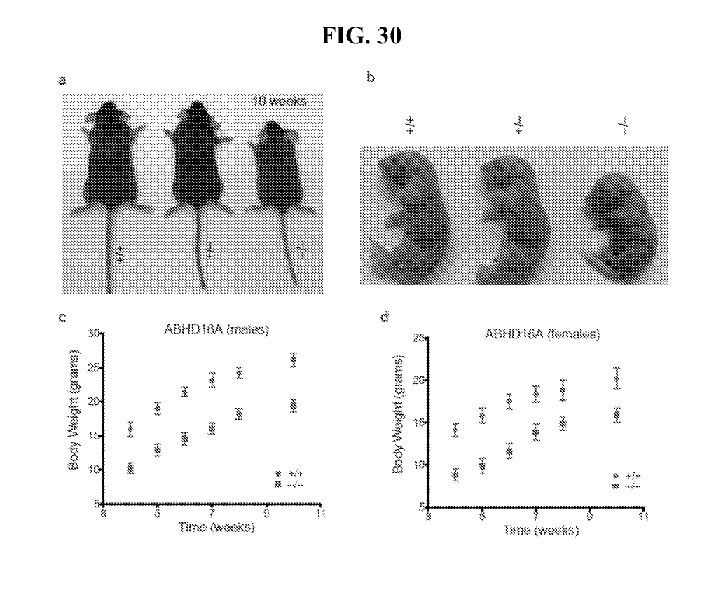

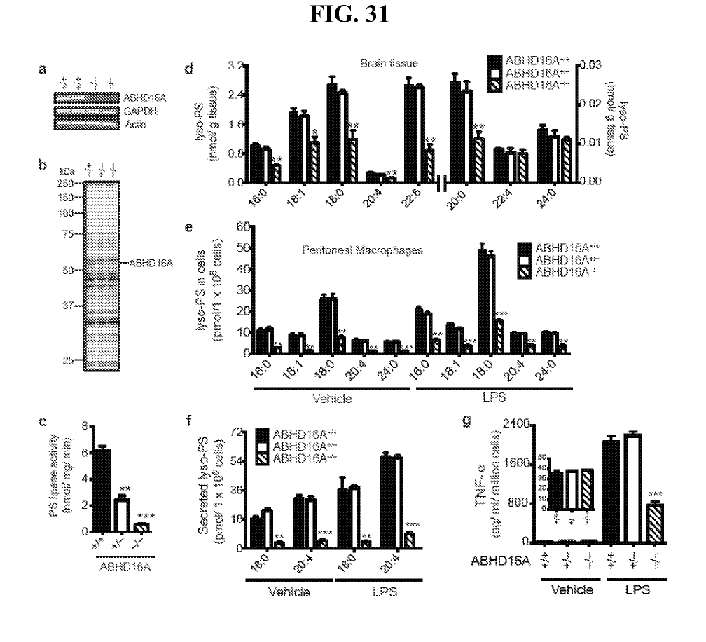

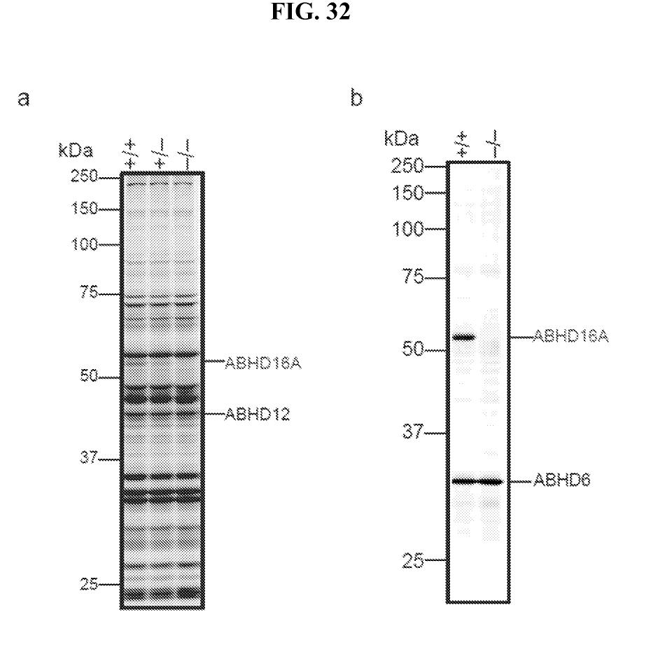

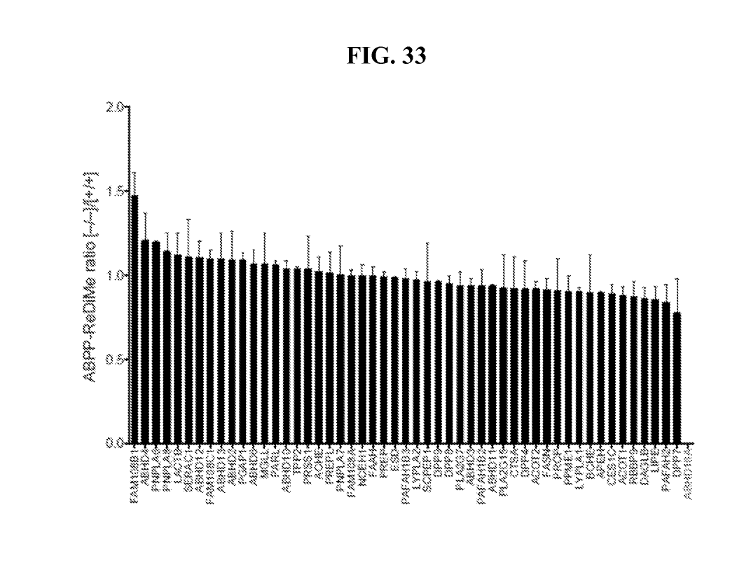

While active in cells, the first-generation ABHD16A inhibitors were not suitable for animal studies, presumably due to their limited bio-distribution and/or stability in vivo. To more directly address the role of ABHD16A in vivo, an ABHD16A^{-/-} mouse model was established. ABHD16A^{+/-} mice on a C57Bl/6 genetic background were acquired from the Wellcome Trust Sanger Institute (see Example 22) and bred to produce ABHD16A-animals, which were identified by PCR-based genotyping (FIG. 29). ABHD16A^{-/-} mice were born at a much lower frequency than expected for Mendelian distribution (54 ABHD16A^{+/+} pups, 87 ABHD16A^{+/-} pups, 10 ABHD16^{-/-} pups) and were ˜30% smaller than ABHD16A^{+/+} or ^{+/-} mice throughout development and life (FIG. 30). Despite their smaller size, ABHD16A^{-/-} mice appeared normal in their cage behavior, and no evidence of increased postnatal lethality in these animals was observed. RT-PCR and ABPP analysis of brain tissue confirmed loss of ABHD16A mRNA expression (FIG. 31a) and protein activity (detected by either the FP-rhodamine (FIG. 31b) or WHP01 (FIG. 32) probe) in ABHD16A mice, respectively. Also confirmed was the loss of ABHD16A activity by combining ABPP with a quantitative MS-based proteomic analysis using reductive dimethylation methods (see Example 28), which revealed complete absence of ABHD16A signals in ABHD16A^{-/-} brains and no changes in the signals for the 40+ other brain serine hydrolases detected in this analysis (FIG. 33).

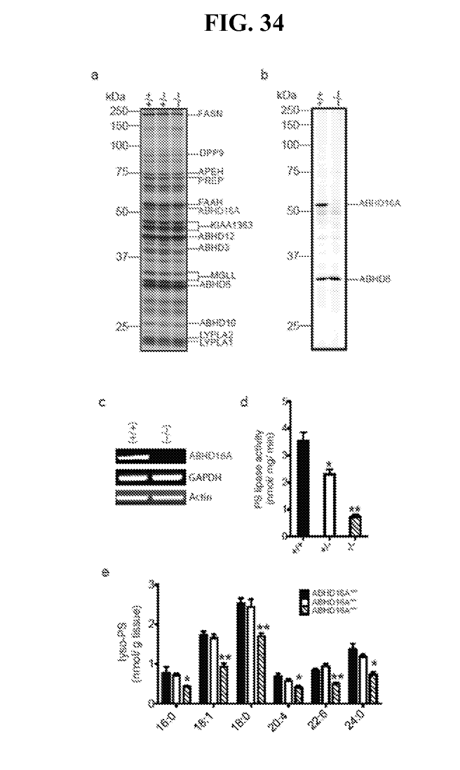

The PS lipase activity of brain membrane lysates from ABHD16^{-/-} mice was greatly decreased compared to ABHD16A^{+/+} and ^{+/-} lysates (FIG. 31c). The brain lipid profiles for ABHD16A^{+/+}, ^{+/-}, and ^{-/-} mice were evaluated, and it was found that ABHD16A^{-/-} mice exhibited substantial reductions in most of the measured lyso-PSs (FIG. 31d). Similar reductions in lyso-PSs were found in spinal cord of ABHD16A^{-/-} mice (FIG. 34), which also exhibited lower PS lipase activity compared to ABHD16A^{+/+} mice (FIG. 34). No changes in CNS lyso-PSs were detected in ABHD16A^{+/-} mice (FIG. 31d and FIG. 34). No changes in PSs or other (lyso)phospholipids in CNS tissues from ABHD16A^{-/-} mice were observed (data not shown).

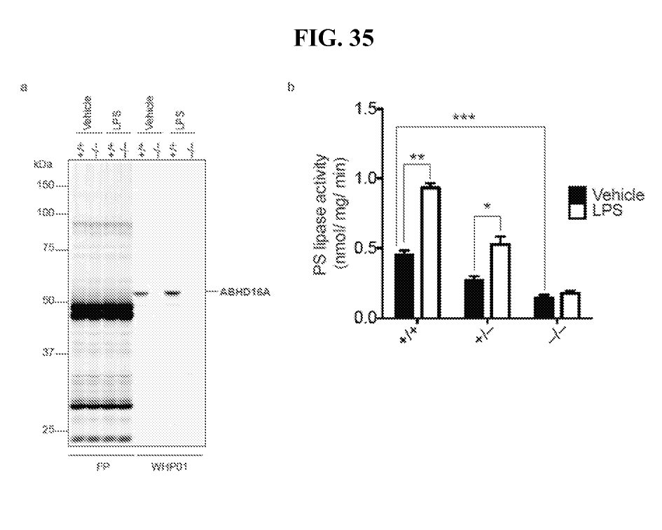

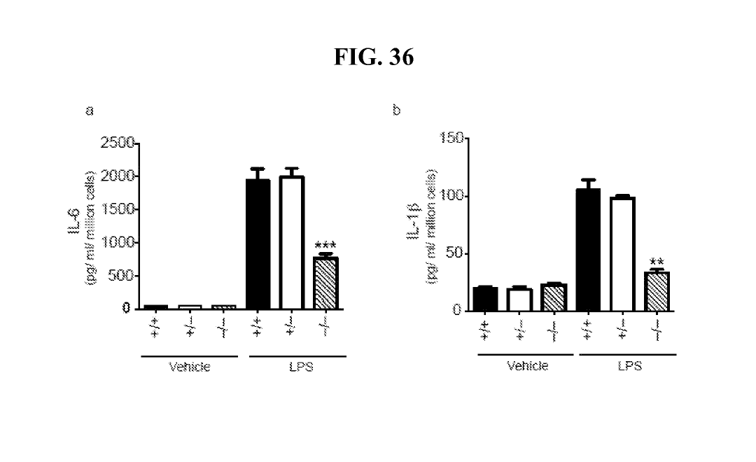

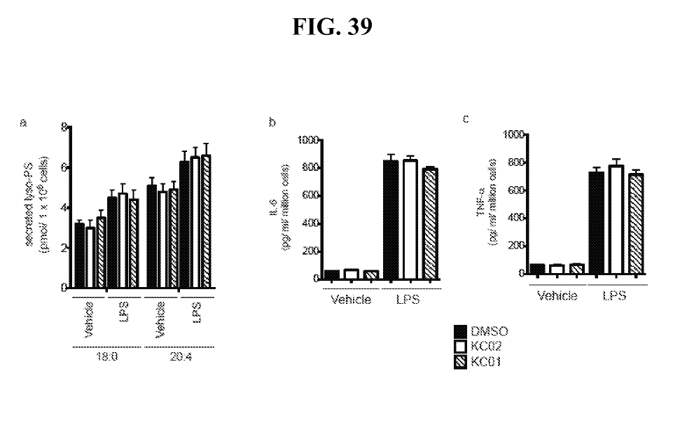

PS metabolism was also examined in thioglycollate-elicited peritoneal macrophages from ABHD16A^{+/+}, ^{+/-}, and ^{-/-} mice. ABPP analysis with the WHP01 probe confirmed loss of ABHD16A activity in ABHD16A^{-/-} macrophages, which also showed greatly reduced PS lipase activity that was no longer elevated by LPS treatment (FIG. 35). ABHD16A^{-/-} macrophages exhibited a corresponding ˜80% reduction in cellular (FIG. 31e) and secreted (FIG. 31f) lyso-PSs under either basal or LPS-stimulated conditions. No changes in PSs, free fatty acids, eicasonoids (PGE2, PGD2, LTB4 or TXB2) or other (lyso)phospholipids were detected in ABHD16A^{-/-} macrophages when compared to the ABHD16A^{+/+} or ^{+/-} macrophages (data not shown). Also assessed were the cytokine profiles of ABHD16A^{+/+}, ^{+/-}, and ^{-/-} macrophages, and it was found that LPS-induced cytokine release was substantially blunted in ABHD16A^{-/-} macrophages (FIG. 31g and FIG. 36). Basal cytokine profiles were unaffected in ABHD16A^{-/-} macrophages (FIG. 31g and FIG. 36). To gain further confidence that KC01 produced its pharmacological effects by blocking ABHD16A, macrophages from ABHD16A^{-/-} mice were treated with KC01 and KC02. No changes were observed under basal conditions or LPS stimulation in cellular or secreted lyso-PS and other measured lipids, or in secreted proinflammatory cytokines (IL-6 and TNF-α), in either KC01- or KC02-treated ABHD16A^{-/-} macrophages (FIG. 39).
These studies on ABHD16A^{-/-} mice corroborate the pharmacological experiments with ABHD16A inhibitors and, together, provide strong evidence that ABHD16A is a principal lyso-PS biosynthetic enzyme in both the CNS and macrophages, and, in the latter cell type, ABHD16A functions as part of a dynamic interplay with ABHD12 to regulate lyso-PS tone and inflammatory responses.

The pharmacological and genetic evidence of the examples described herein compellingly suggests that ABHD16A is a major enzyme responsible for generating lyso-PSs in mammalian cells and in vivo. That PS lipids were, for the most part, unchanged in ABHD16A-disrupted cells or tissues could indicate alternative metabolic routes (e.g., PS decarboxylation) control bulk PS content in vivo. One exception was the K562 cells, where chronic ABHD16A depletion by RNA-interference resulted in, not only reductions in lyso-PS content, but also elevations in PSs. It therefore remains possible that, in certain cell types or under specific conditions, ABHD16A could coordinately regulate both PS and lyso-PS lipids.

The data disclosed herein provide substantial experimental evidence that ABHD16A, along with its lyso-PS products, and the lyso-PS lipase ABHD12, constitute a novel lipid signaling network that is dynamically regulated by, and in turn contributes to, macrophage inflammatory responses. While the precise receptors involved in transmitting lyso-PS signals in macrophages (or the CNS) are not yet known, previous studies invoke both innate immune receptors and GPCRs as candidates. Interestingly, GPR174, a lyso-PS-activated GPCRs, has recently been implicated by human genetics studies as a contributory factor to the autoimmune syndrome Grave's disease. Notably, while this data and other studies point to an immunostimulatory function for lyso-PSs, other investigations have uncovered an additional role for these lipids as resolving signals in inflammation. The discovery of the lyso-PS regulatory enzymes ABHD16A and ABHD12 offers genetic and pharmacological entry points to decipher the contributions of lyso-PS pathways to (neuro)immunology and other biological processes.

Several of these findings provide evidence for an interplay between ABHD16A and ABHD12 in the potential regulation of PHARC. First, the ABHD16A inhibitor KC01 reversed the elevated lyso-PS production observed in ABHD12-null cells derived from a PHARC subject. ABHD16A^{-/-} mice also showed lower lyso-PS content in the CNS that ran counter to the heightened lyso-PS profile of ABHD12^{-/-} mice. Moreover, the cerebellum, which was the brain region that exhibits the most dramatic changes in lyso-PSs and microglial activation in ABHD12^{-/-} mice and undergoes atrophy in PHARC patients, is enriched in ABHD16A expression (FIG. 3c).

These findings demonstrate that ABHD16A is a major lyso-PS-producing enzyme in mammalian systems and disruption of this pathway attenuates inflammatory processes that may contribute to PHARC and other (neuro)immunological disorders.

TABLE 2

| | | Lipase activity | | | | Student's t- |
|---|---|---|---|---|---|---|
| | | (nmol/mg/min) | | | | test p-value |
| Substrate | Products | ABHD16A | | Mock | | ABHD16 A vs |
| (100 μM) | detected^{a} | Mean | SEM | Mean | SEM | Mock |
| PS 17:0/20:4 | Lyso-PS 17:0, | 25.8 | 1.1 | 1.8 | 0.3 | 0.0032 |
| | FFA 20:4 | | | | | |
| PS 16:0/18:1 | Lyso-PS 16:0, | 30.1 | 1.7 | 2.5 | 0.5 | 0.0026 |
| | FFA 18:1 | | | | | |
| PS 18:0/18:2 | Lyso-PS 18:0, | 27.9 | 1.2 | 2.2 | 0.2 | 0.0023 |
| | FFA 18:2 | | | | | |
| PC 17:0/20:4 | Lyso-PC 17:0, | 12.9 | 1.0 | 3.0 | 0.0 | 0.0103 |
| | FFA 20:4 | | | | | |
| PE 17:0/20:4 | Lyso-PE 17:0, | 11.1 | 1.1 | 3.9 | 0.1 | 0.0217 |
| | FFA 20:4 | | | | | |
| PI 16:0/18:1 | Lyso-PI 16:0, | 4.5 | 0.1 | 1.7 | 0.3 | 0.0097 |
| | FFA 18:1 | | | | | |
| PG 16:0/18:1 | Lyso-PG 16:0, | 12.5 | 0.7 | 3.8 | 0.3 | 0.0078 |
| | FFA 18:1 | | | | | |
| PA 17:0/20:4 | Lyso-PA 17:0, | 7.4 | 0.8 | 3.6 | 0.5 | 0.0294 |
| | FFA 20:4 | | | | | |
| DAG 16:0/18:1 | MAG 16:0, | 1.8 | 0.6 | 0.8 | 0.2 | 0.2426 |
| | FFA 18:1 | | | | | |
| Lyso-PS 18:1 | FFA 18:1 | 1.2 | 0.1 | 0.6 | 0.0 | 0.0442 |
| Lyso-PC 18:1 | FFA 18:1 | 1.7 | 0.1 | 1.3 | 0.0 | 0.0241 |
| Lyso-PE 18:1 | FFA 18:1 | 0.8 | 0.0 | 0.2 | 0.0 | 0.0656 |
| Lyso-PI 18:1 | FFA 18:1 | 0.5 | 0.0 | 0.5 | 0.0 | 0.7057 |
| Lyso-PG 18:1 | FFA 18:1 | 0.7 | 0.0 | 0.7 | 0.0 | 0.5995 |
| Lyso-PA 18:1 | FFA 18:1 | 0.7 | 0.1 | 0.5 | 0.0 | 0.3278 |
| MAG 18:1 | FFA 18:1 | 0.7 | 0.0 | 0.2 | 0.0 | 0.2731 |

^{a}Internal standard was FFA 17:1.

We claim:

1. A compound of Formula (II): 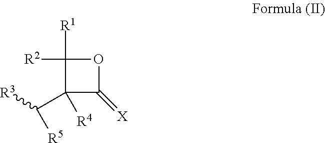
wherein:
X is O or CH₂;
R¹ is alkyl or -alkylene-(optionally substituted aryl);
R² is H;
R³ is optionally substituted aryl, —(CH₂)ₙ—CH₃, —(CH₂)ₙ-(optionally substituted aryl), —(CH₂)ₙ—C(O)NR⁶R⁷, or —(CH₂)ₙ—SO₂NR⁶R⁷;
R⁴ and R⁵ are both H; or R⁴ and R⁵ together form a direct bond to provide a double bond;
R⁶ is H or alkyl;
R⁷ is H or alkyl; and
n is 1, 2, 3, 4, 5, or 6;
or a solvate, hydrate, tautomer, N-oxide, stereoisomer, or pharmaceutically acceptable salt thereof.

2. The compound of claim 1, wherein R¹ is alkyl or -alkylene-(optionally substituted phenyl).

3. The compound of claim 1 or claim 2, wherein R³ is optionally substituted phenyl, —(CH₂)ₙ—CH₃, —(CH₂)ₙ-(optionally substituted phenyl), —(CH₂)ₙ—C(O)NR⁶R⁷, or —(CH₂)ₙ—SO₂NR⁶R⁷.

4. The compound of any one of claims 1-3, wherein R⁶ and R⁷ are both H.

5. The compound of any one of claims 1-4, wherein R⁴ and R⁵ are both H.

6. The compound of any one of claims 1-4, wherein R⁴ and R⁵ together form a direct bond to provide a double bond.

7. The compound of any one of claims 1-6, wherein X is O.

8. The compound of any one of claims 1-6, wherein X is CH₂.

9. A compound of Formula (III): 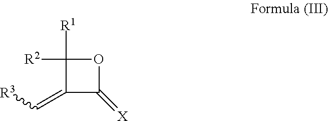
wherein:
X is O or CH₂;
R¹ is alkyl or -alkylene-(optionally substituted aryl);
R² is H;
R³ is optionally substituted aryl, —(CH₂)ₙ—CH₃, —(CH₂)ₙ-(optionally substituted aryl),
—(CH₂)ₙ—C(O)NR⁶R⁷, or —(CH₂)ₙ—SO₂NR⁶R⁷;
R⁶ is H or alkyl;
R⁷ is H or alkyl; and
n is 1, 2, 3, 4, 5, 6, 7, 8, 9, 10, 11, 12, 13, 14, 15, 16, 17, 18, 19, or 20;
or a solvate, hydrate, tautomer, N-oxide, stereoisomer, or pharmaceutically acceptable salt thereof.

10. The compound of claim 9, wherein R¹ is alkyl or -alkylene-(optionally substituted phenyl).

11. The compound of claim 9 or claim 10, wherein R³ is optionally substituted phenyl, —(CH₂)ₙ—CH₃, —(CH₂)ₙ-(optionally substituted phenyl), —(CH₂)ₙ—C(O)NR⁶R⁷, or —(CH₂)ₙ—SO₂NR⁶R⁷.

12. The compound of any one of claims 9-11, wherein R⁶ and R⁷ are both H.

13. The compound of any one of claims 9-12, wherein X is O.

14. The compound of any one of claims 9-12, wherein X is CH₂.

15. A compound of Formula (IV): 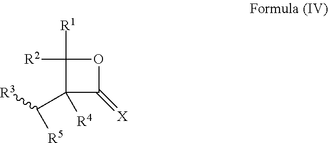
wherein:
X is O or CH₂;
R¹ is alkyl or -alkylene-(phenyl);
R² is H;
R³ is phenyl, —(CH₂)ₙ—CH₃, —(CH₂)ₙ-(phenyl), —(CH₂)ₙ—C(O)NH₂, or —(CH₂)ₙ—SO₂NH₂;
R⁴ and R⁵ are both H; or R⁴ and R⁵ together form a direct bond to provide a double bond; and
n is 1, 2, 3, 4, 5, or 6;
or a solvate, hydrate, tautomer, N-oxide, stereoisomer, or pharmaceutically acceptable salt thereof.

16. The compound of claim 15, wherein R⁴ and R⁵ are both H.

17. The compound of claim 15, wherein R⁴ and R⁵ together form a direct bond to provide a double bond.

18. The compound of any one of claims 15-17, wherein X is O.

19. The compound of any one of claims 15-17, wherein X is CH₂.

20. A compound of Formula (V): 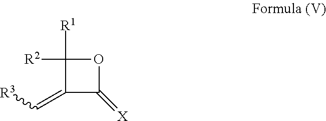
wherein:
X is O or CH₂;
R¹ is alkyl or -alkylene-(phenyl);
R² is H;
R³ is phenyl, —(CH₂)ₙ—CH₃, —(CH₂)ₙ-(phenyl), —(CH₂)ₙ—C(O)NH₂, or —(CH₂)ₙ—SO₂NH₂;
n is 1, 2, 3, 4, 5, 6, 7, 8, 9, 10, 11, 12, 13, 14, 15, 16, 17, 18, 19, or 20;
or a solvate, hydrate, tautomer, N-oxide, stereoisomer, or pharmaceutically acceptable salt thereof.

21. The compound of claim 20, wherein X is O.

22. The compound of claim 20, wherein X is CH₂.

23. A compound of Formula (VI): 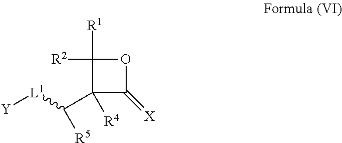
wherein:
X is O or CH₂;
R¹ is alkyl or -alkylene-(optionally substituted aryl);
R² is H;
R⁴ and R⁵ are both H; or R⁴ and R⁵ together form a direct bond to provide a double bond;
L¹ is a linker; and
Y is a fluorophore;
or a solvate, hydrate, tautomer, N-oxide, stereoisomer, or pharmaceutically acceptable salt thereof.

24. The compound of claim 23, wherein X is O.

25. The compound of claim 23, wherein X is CH₂.

26. The compound of any one of claims 23-25, wherein R⁴ and R⁵ are both H.

27. The compound of any one of claims 23-25, wherein R⁴ and R⁵ together form a direct bond to provide a double bond.

28. The compound of any one of claim 23-27, wherein Y is selected from fluorescein, 6-FAM, rhodamine, Texas Red, California Red, iFluor594, carboxytetramethylrhodamine, tetramethylrhodamine, a carboxyrhodamine, carboxyrhodamine 6F, carboxyrhodol, carboxyrhodamine 110, Cascade Blue, Cascade Yellow, coumarin, Cy2®, Cy3®, Cy3.5®, Cy5®, Cy5.5®, Cy7®, Cy-Chrome, DyLight 350, DyLight 405, DyLight 488, DyLight 549, DyLight 594, DyLight 633, DyLight 649, DyLight 680, DyLight 750, DyLight 800, phycoerythrin, PerCP (peridinin chlorophyll-a Protein), PerCP-Cy5.5, JOE (6-carboxy-4′,5′-dichloro-2′,7′-dimethoxyfluorescein), NED, ROX (5-(and-6-)-carboxy-X-rhodamine), HEX, Lucifer Yellow, Marina Blue, Oregon Green 488, Oregon Green 500, Oregon Green 514, Alexa Fluor® 350, Alex Fluor® 430, Alexa Fluor® 488, Alexa Fluor® 532, Alexa Fluor® 546, Alexa Fluor® 568, Alexa Fluor® 594, Alexa Fluor® 633, Alexa Fluor® 647, Alexa Fluor® 660, Alexa Fluor® 680, 7-amino-4-methylcoumarin-3-acetic acid, BODIPY® FL, BODIPY® FL-Br₂, BODIPY® 530/550, BODIPY® 558/568, BODIPY® 630/650, BODIPY® 650/665, BODIPY® R6G, BODIPY® TMR, BODIPY® TR, conjugates thereof, derivatives thereof, analogs thereof, and combinations thereof.

29. A compound selected from: 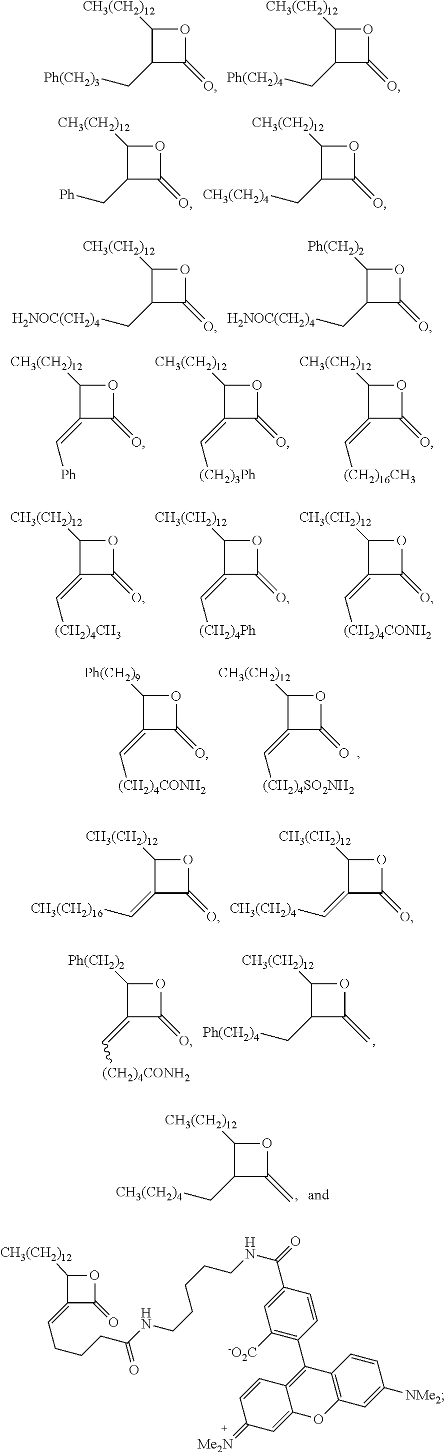
or a solvate, hydrate, tautomer, N-oxide, stereoisomer, or a pharmaceutically acceptable salt thereof.

30. A pharmaceutical composition comprising a compound of any one of claims 1-29, or a solvate, hydrate, tautomer, N-oxide, stereoisomer, or a pharmaceutically acceptable salt thereof, and at least one pharmaceutically acceptable excipient.

31. A method of treating PHARC comprising administering to a subject in need thereof a therapeutically effective amount of an ABHD16A inhibitor.

32. A method of treating PHARC comprising administering to a subject in need thereof a therapeutically effective amount of a compound of any one of claims 1-22.

33. A method of treating PHARC comprising administering to a subject in need thereof a therapeutically effective amount of a compound selected from: 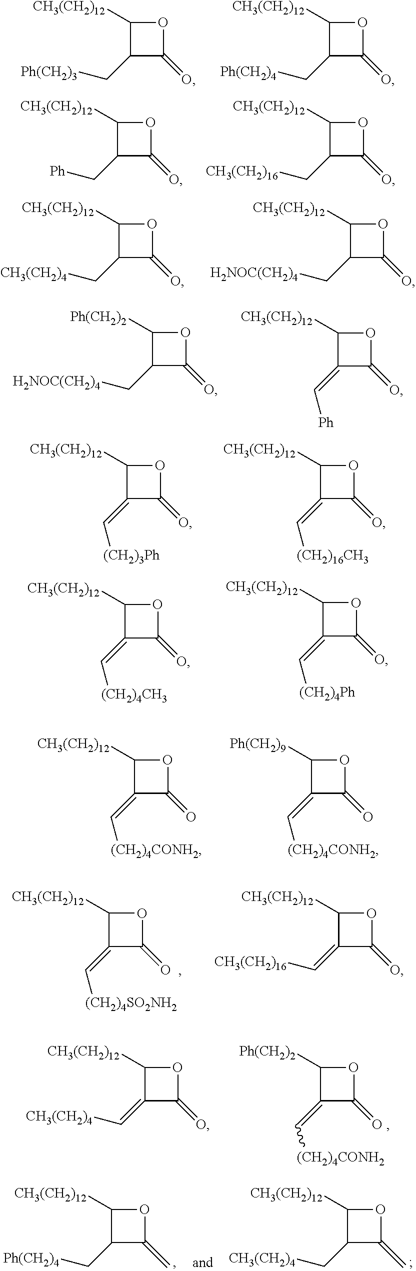
or a solvate, hydrate, tautomer, N-oxide, stereoisomer, or a pharmaceutically acceptable salt thereof.

34. A method of treating a neuroinflammatory disease comprising administering to a subject in need thereof a therapeutically effective amount of an ABHD16A inhibitor.

35. A method of treating a neuroinflammatory disease comprising administering to a subject in need thereof a therapeutically effective amount of a compound of any one of claims 1-22.

36. A method of treating a neuroinflammatory disease comprising administering to a subject in need thereof a therapeutically effective amount of a compound selected from: 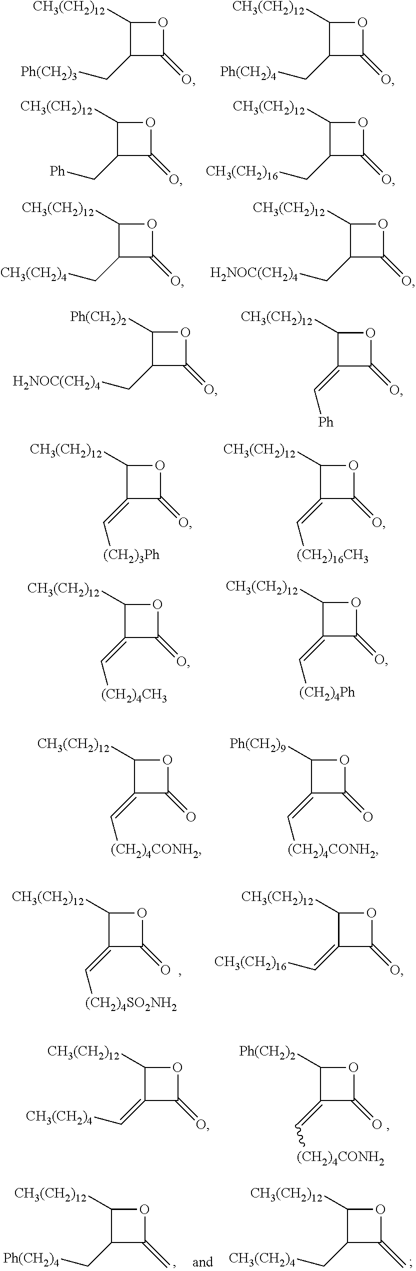
or a solvate, hydrate, tautomer, N-oxide, stereoisomer, or a pharmaceutically acceptable salt thereof.

37. A method of inhibiting ABHD16A in mammalian cells, the method comprising contacting the mammalian cells with a compound of any one of claims 1-28.

38. A method of inhibiting ABHD16A in mammalian cells, the method comprising contacting the mammalian cells with a compound selected from: 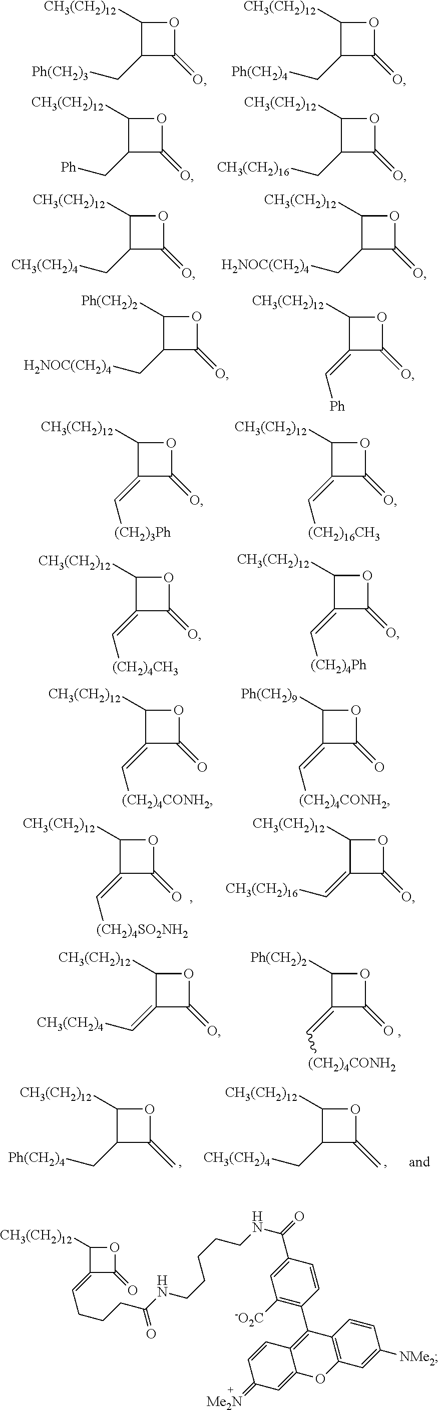
or a solvate, hydrate, tautomer, N-oxide, stereoisomer, or a pharmaceutically acceptable salt thereof.

39. A method of reducing cellular and/or secreted levels of pro-inflammatory lysophosphatidylserine lipids in mammalian cells, the method comprising contacting the mammalian cells with an ABHD16A inhibitor.

40. A method of reducing cellular and/or secreted levels of pro-inflammatory lysophosphatidylserine lipids in mammalian cells, the method comprising contacting the mammalian cells with a compound of any one of claims 1-28.

41. A method of reducing cellular and/or secreted levels of pro-inflammatory lysophosphatidylserine lipids in mammalian cells, the method comprising contacting the mammalian cells with a compound selected from: 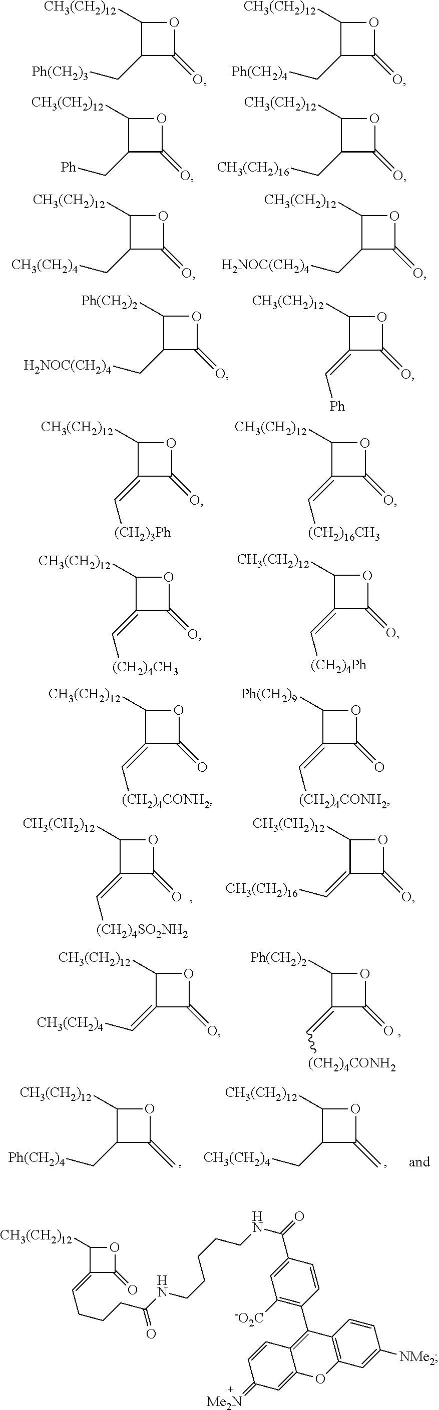
or a solvate, hydrate, tautomer, N-oxide, stereoisomer, or a pharmaceutically acceptable salt thereof.

42. The method of any one of claims 37-41, wherein the method is an in vivo method.

43. The method of any one of claims 37-41, wherein the method is an in vitro method.
